(19) United States
(12) Patent Application Publication
(10) Pub. No.: US 2022/0186218 A1
(43) Pub. Date: Jun. 16, 2022

(54) METHODS AND COMPOSITIONS FOR CORRECTED ABERRANT SPLICE SITES

(71) Applicants: THE CHILDREN'S MEDICAL CENTER CORPORATION, Boston, MA (US); UNIVERSITY OF MASSACHUSETTS, Boston, MA (US)

(72) Inventors: Daniel E. BAUER, Cambridge, MA (US); Shuqian XU, Brookline, MA (US); Scot A. WOLFE, Winchester, MA (US); Kevin LUK, Worcester, MA (US)

(73) Assignees: THE CHILDREN'S MEDICAL CENTER CORPORATION, Boston, MA (US); UNIVERSITY OF MASSACHUSETTS, Boston, MA (US)

(21) Appl. No.: 17/425,689

(22) PCT Filed: Jan. 24, 2020
(86) PCT No.: PCT/US20/15022

§ 371 Date: Jul. 23, 2021

Related U.S. Application Data
(60) Provisional application No. 62/796,288, filed on Jan. 24, 2019.

(51) Int. Cl. C12N 15/113; C07K 14/805; C12N 9/22; C12N 15/11

(52) U.S. Cl. CPC C12N 15/113; C07K 14/805; C12N 2310/20; C12N 15/111; C12N 9/22

(57) ABSTRACT

Provided herein are ribonucleoprotein (RNP) complexes comprising a DNA-targeting endonuclease Cas (CRISPR-associated) protein and a guide RNA (gRNA) that that targets and hybridizes to the β-Globin gene. In one embodiment, the Cas protein is Cas9 and the gRNA comprises the sequence of SEQ ID NO: 1. In one embodiment, the Cas protein is Cas12a and the gRNA comprises the sequence of SEQ ID NO: 3.

### CROSS-REFERENCE TO RELATED APPLICATION

This application is an International Application which designated the U.S., and which claims the benefit under 35 U.S.C. § 119(e) of U.S. Provisional Application No. 62/796,288 filed on Jan. 24, 2019, the contents of which are incorporated herein by reference in their entireties.

### GOVERNMENT SUPPORT

This invention was made with Government support under Grant No. R01GM115911 awarded by the National Institutes of Health. The Government has certain rights in the invention.

### BACKGROUND

Normal adult hemoglobin comprises four globin proteins, two of which are alpha (α) proteins and two of which are beta (β) proteins. During mammalian fetal development, particularly in humans, the fetus produces fetal hemoglobin, which comprises two gamma (γ)-globin proteins instead of the two β-globin proteins. During the neonatal period, a globin switch occurs, referred to as the “fetal switch”, at which point, erythroid precursors switch from making predominantly γ-globin to making predominantly β-globin. The developmental switch from production of predominantly fetal hemoglobin or HbF (α₂γ₂) to production of adult hemoglobin or HbA (α₂β₂) begins at about 28 to 34 weeks of gestation and continues shortly after birth until HbA becomes predominant. This switch results primarily from decreased transcription of the gamma-globin genes and increased transcription of beta-globin genes. On average, the blood of a normal adult contains less than 1% HbF, though residual HbF levels have a variance of over 20 fold in healthy adults and are genetically controlled.

Hemoglobinopathies encompass a number of anemias of genetic origin in which there is a decreased production and/or increased destruction (hemolysis) of red blood cells (RBCs). These also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickle) hemoglobin (HbS). HbS is prone to polymerization, particularly under deoxygenated conditions. HbS RBCs are more fragile than normal RBCs and undergo hemolysis more readily, leading eventually to anemia.

The β-thalassemias are a genetically heterogeneous set of conditions in which various mutations at HBB result in partial (β⁺) or complete (β⁰) loss of β-globin expression. Several of the most common mutant alleles disrupt HBB splicing through the creation of aberrant splice sites. It will be important to uncover therapeutic methods for correcting these aberrant splice sites in order to treat β-thalassemia patients.

### SUMMARY

One aspect described herein provides a ribonucleoprotein (RNP) complex comprising a DNA-targeting endonuclease Cas (CRISPR-associated) protein and a guide RNA comprising the sequence of SEQ ID NO: 1 or 3 that targets and hybridizes to a target sequence on a DNA molecule.

In one embodiment of any aspect described herein, the CRISPR enzyme is a type II CRISPR system enzyme.

In one embodiment of any aspect described herein, the CRISPR enzyme is a Cas enzyme. Exemplary Cas proteins include Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

In one embodiment of any aspect described herein, the Cas protein is Cas9 or Cas12a.

In one embodiment of any aspect described herein, the RNP complex provided herein is for use in altering the genetic sequence of a gene.

In one embodiment of any aspect described herein, altering is a nucleotide deletion, insertion or substitution of the genetic sequence.

In one embodiment of any aspect described herein, altering promotes proper intron splicing of a gene.

In one embodiment of any aspect described herein, altering is correcting a genetic mutation in a gene.

In one embodiment of any aspect described herein, the gene is β-Globin.

In one embodiment of any aspect described herein, the genetic mutation is IVS1-110G>A or IVS2-654C>T.

In one embodiment of any aspect described herein, the genetic mutation is selected from those listed in Table 2.

In one embodiment of any aspect described herein, the guide RNA comprises a sequence selected from those listed in Table 2.

In one embodiment of any aspect described herein, the RNP complex provided herein further comprising a crRNA/tracrRNA sequence.

In one embodiment of any aspect described herein, the RNP complex provided herein is for use in an ex vivo method of producing a progenitor cell or a population of progenitor cell wherein the cells or the differentiated progeny thereof have an altered genetic sequence.

In one embodiment of any aspect described herein, the RNP complex provided herein is for use in an ex vivo method of producing a progenitor cell or a population of progenitor cell wherein the cells or the differentiated progeny thereof have corrected a IVS1-110G>A or IVS2-654C>T mutation.

In one embodiment of any aspect described herein, the RNP complex provided herein is for use in an ex vivo method of producing a progenitor cell or a population of progenitor cell wherein the cells or the differentiated progeny thereof have at least one genetic modification in the β-Globin gene.

In one embodiment of any aspect described herein, the RNP complex provided herein is for use in an ex vivo method of producing an isolated genetic engineered human cell or a population of genetic engineered human cells having an altered genetic sequence.

In one embodiment of any aspect described herein, the RNP complex provided herein is for use in an ex vivo method of producing an isolated genetic engineered human cell or a population of genetic engineered human cells which have corrected a IVS1-110G>A or IVS2-654C>T mutation.

In one embodiment of any aspect described herein, the RNP complex provided herein is for use in an ex vivo method of producing an isolated genetic engineered human cell or a population of genetic engineered human cells having at least one genetic modification in the β-Globin gene.

In one embodiment of any aspect described herein, the cell is a hematopoietic progenitor cell or a hematopoietic stem cell.

In one embodiment of any aspect described herein, the hematopoietic progenitor is a cell of the erythroid lineage.

In one embodiment of any aspect described herein, the isolated human cell is an induced pluripotent stem cell.

In one embodiment of any aspect described herein, the IVS1-110G>A or IVS2-654C>T mutation is present in the β-Globin gene

Another aspect provided herein provides a composition comprising any of the RNP complexes described herein.

Yet another aspect provided herein provides a composition comprising any of the progenitor cells or population thereof provided herein, or any of the isolated genetic engineered human cell or population thereof provided herein.

In one embodiment of any aspect described herein, the composition further comprises a pharmaceutically acceptable carrier.

In one embodiment of any aspect described herein, any of the compositions thereof are for use in an ex vivo method of producing a progenitor cell or a population of progenitor cells wherein the cells or the differentiated progeny therefrom have an altered genetic sequence, have corrected a IVS1-110G>A or IVS2-654C>T mutation, and/or have at least one genetic modification in the β-Globin gene.

In one embodiment of any aspect described herein, any of the compositions thereof are for use in an ex vivo method of producing an isolated genetic engineered human cell or a population of progenitor cells having an altered genetic sequence, having a corrected a IVS1-110G>A or IVS2-654C>T mutation, and/or having at least one genetic modification in the β-Globin gene.

Another aspect provided herein provides a method for correcting an isolated progenitor cell or a population of isolated progenitor cells having a IVS1-110G>A or IVS2-654C>T mutation in the 13-Globin gene, the method comprising contacting an isolated progenitor cell with an effective amount of any of the RNP complexes described herein, or any of the compositions described herein, whereby the contacted cells or the differentiated progeny cells therefrom have corrected the IVS1-110G>A or IVS2-654C>T mutation in the β-Globin gene.

In one embodiment of any aspect described herein, the isolated progenitor cell is a hematopoietic progenitor cell or a hematopoietic stem cell.

In one embodiment of any aspect described herein, the hematopoietic progenitor is a cell of the erythroid lineage.

In one embodiment of any aspect described herein, the isolated progenitor cell is an induced pluripotent stem cell.

In one embodiment of any aspect described herein, the isolated progenitor cell is contacted ex vivo or in vitro.

Another aspect provided herein provides a population of any of the genetically edited progenitor cells produced by any of the methods described herein.

In one embodiment of any aspect described herein, the genetically edited human cells are isolated.

Another aspect provided herein provides a composition comprising any of the isolated genetically edited human cells described herein.

Another aspect provided herein provides a method of treating a disease associated with IVS1-110G>A or IVS2-654C>T mutation in the β-Globin gene, the method comprising, administering to a subject in need thereof any of the RNP complexes provided herein, any of the compositions provided herein, or any of population of genetically edited progenitor cells of claims 35-36.

In one embodiment of any aspect described herein, the disease is thalassemia or β-thalassemia.

Another aspect provided herein provides a RNP complex comprising a DNA-targeting endonuclease Cas9 protein and a guide RNA comprising the sequence of SEQ ID NO: 1 that targets and hybridizes to a target sequence on a DNA molecule.

Another aspect provided herein provides a RNP complex comprising a DNA-targeting endonuclease Cas12a protein and a guide RNA comprising the sequence of SEQ ID NO: 3 that targets and hybridizes to a target sequence on a DNA molecule.

In one embodiment of any aspect described herein, targeting and hybridizing corrects a IVS1-110G>A or mutation is present in the β-Globin gene

In one embodiment of any aspect described herein, targeting and hybridizing corrects a IVS2-654C>T mutation is present in the β-Globin gene.

### DEFINITIONS

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given composition, cell or RNP complex described herein) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level (e.g. the absence of a given composition, cell or RNP complex described herein), for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., hemaglobinopathies or cancer. A subject can be male or female.

A subject can be one who has been previously diagnosed with or identified as suffering from or having a condition in need of treatment (e.g. a hemoglobinopathy, such as β-thalassemia) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

In one embodiment, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of the genomic sequence of the cell. An “engineered cell” can refer to a cell with an added, deleted and/or altered genomic sequence. The term “cell” or “engineered cell” and their grammatical equivalents as used herein can refer to a cell of human or non-human animal origin.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physicochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligan-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. For example, a functional fragment described herein would retain at least 50% of the CRISPR enzyme function. One skilled in the art can assess the function of a CRISPR enzyme using standard techniques, for example those described herein below. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking Conversely, cysteine bond(s) can be added to a polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “pharmaceutical composition” refers to the active agent (e.g., an RNP complex or edited cell described herein) in combination with a pharmaceutically acceptable carrier e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

As used herein, the term “administering” refers to the placement of a therapeutic (e.g., an engineered cell or RNP described herein) or pharmaceutical composition as disclosed herein into a subject by a method or route which results in at least partial delivery of the agent at a desired site. Pharmaceutical compositions comprising agents as disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined within the description of the various aspects and embodiments of the technology of the following.

### BRIEF DESCRIPTION OF DRAWINGS

FIGS. 1A-1J show therapeutic gene editing of IVS1-110G>A. (FIG. 1A) Schema of IVS1-110G>A mutation within HBB intron land therapeutic editing strategy. (FIG. 1B) Indicated donors and sgRNAs used for therapeutic editing. 5 days after RNP electroporation, amplicon deep sequencing was performed on the SpCas9-treated cells. Following sequence analysis, alleles were classified as edited, unedited IVS1-110G>A or unedited IVS1-110G. (FIG. 1C) Nucleotide quilt showing indels and substitutions at each position around IVS1-110 for indicated donors and SpCas9 RNP treatment groups. β⁺β⁰_{#1} with sgAAVS1 shown as a representative example of an unedited IVS1-110G>A heterozygous donor and β⁺β⁺ with sgAAVS1 as a representative example of an unedited IVS1-110G>A homozygous/hemizygous donor. (FIG. 1D) Reverse transcription PCR from erythroid progeny with primers spanning the exon 1-exon 2 junction, demonstrates abrogation of aberrant (A) and increase in normal (N) splicing after therapeutic editing. (FIG. 1E) RT-qPCR of globin genes shows increase in β-globin relative to β-globin expression in erythroid progeny after therapeutic editing. (FIG. 1F) Hemoglobin HPLC shows increase in the hemoglobin A (HbA) fraction after therapeutic editing. (FIGS. 1G AND 1H) Flow cytometry shows increase in enucleation fraction and cell size of enucleated erythroid cells after therapeutic editing. (FIG. 1I) Reverse transcription PCR from clonal erythroid progeny with primers spanning the exon 1-exon 2 junction. Indel length of edited IVS1-110G>A allele depicted for individual clones. (FIG. 1J) FACS sorting of CD34+CD38+ hematopoietic progenitor (HPC) or CD34+CD38−CD90+CD45RA-hematopoietic stem cell (HSC) enriched populations 2 hours after therapeutic editing of β⁺β⁺ donor, which was 24 hours after CD34+ HSPC isolation. Indel analysis performed 5 days after sorting.

FIGS. 2A-2H shows therapeutic gene editing of IVS2-654C>T. (FIG. 2A) Schema of IVS2-654C>T mutation and therapeutic editing strategy. Cut site is shown at midpoint of expected Cas12a staggered cleavage. (FIG. 2B) Indicated donors and crRNAs used for therapeutic editing. 5 days after RNP electroporation, amplicon deep sequencing was performed on the LbCas12a-treated cells. Following sequence analysis, alleles were classified as edited, unedited IVS2-654C>T or unedited IVS2-654C. (FIG. 2C) Nucleotide quilt showing indels and substitutions at each position around IVS2-654 for indicated donors and LbCas12a RNP treatment groups. β⁺β⁺_{#5} with sgAAVS1 shown as a representative example of an unedited IVS2-654C>T heterozygous donor with rs1609812-T/T. β⁺β⁰_{#}4 shown as a donor in which the IVS2-654C/rs1609812-C and IVS2-654C>T/rs1609812-T alleles could be distinguished. (FIG. 2D) Reverse transcription PCR from erythroid progeny with primers spanning the exon 2-exon 3 junction, demonstrates abrogation of aberrant (A) and increase in normal (N) splicing after therapeutic editing. (FIG. 2E) RT-qPCR of globin genes shows increase in β-globin relative to β-globin expression in erythroid progeny after therapeutic editing. (FIG. 2F) Hemoglobin HPLC shows increase in the hemoglobin A (HbA) fraction after therapeutic editing. (FIGS. 2G and 2H) Flow cytometry shows increase in enucleation fraction and cell size of enucleated erythroid cells after therapeutic editing.

FIGS. 3A-3B show allele plots of therapeutic editing at IVS1-110G>A and IVS2-654C>T alleles. Consensus splice acceptor and donor sites are illustrated above the aberrant splice sites. (FIG. 3A) Enumeration of indel type following sgIVS1-110A SpCas9 RNP editing of β⁺β⁰_{#1} aligned to IVS1-110A reference. (FIG. 3B) Enumeration of indel type following crIVS2-654T LbCas12a RNP editing of β⁺β⁰_{#4} aligned to IVS2-654T/rs1609812-T reference.

FIGS. 4A-4B show hemoglobin HPLC traces following therapeutic editing at IVS1-110G>A and IVS2-654C>T alleles. (FIG. 4A) Top shows hemoglobin HPLC traces in erythroid progeny after sgAAVS1 SpCas9 RNP editing and bottom after sgIVS1-110A SpCas9 RNP editing. (FIG. 4B) Top shows hemoglobin HPLC traces in erythroid progeny after crAAVS1 LbCas12a RNP editing and bottom after crIVS2-654T LbCas12a RNP editing. HbA2, HbE, and HbLepore co-migrate.

FIG. 5 shows sorting edited HSC and HPC populations. Representative gating strategy indicating live singlets with CD34+CD38+ (HPC) and CD34+CD38−CD90+CD45RA-immunophenotypes.

FIG. 6 shows GUIDE-Seq for sgIVS1-110A editing by 3×NLS-SpyCas9 in HEK293T cells by plasmid transient transfection in HEK293T cells. Unique read counts at 13 potential off-target sites (OT #), in addition to the on-target IVS1-110A site.

FIG. 7 shows amplicon-seq at GUIDE-seq predicted (OT1-13) and Cas-OFFinder tool (OT14-28) predicted off-target sites in HEK293T by plasmid transient transfection. Indel frequencies by 3×NLS-SpyCas9 at on-target and 28 perspective off-target sites determined by illumina sequencing of PCR amplicons spanning each genomic region.

FIG. 8 shows amplicon-seq at most active validated off-target sites in RNP treated patient CD34 HSPCs. Indel frequencies by 3×NLS-SpyCas9 at on-target and top 4 off-target sites validated by HEK293T experiment determined by illumina sequencing of PCR amplicons spanning each genomic region.

FIG. 9 shows GUIDE-Seq for sgIVS1-110A in HEK293T cells by ribonucleoprotein (RNP) Neon transfection. Unique read counts at 10 new potential off-target sites, in addition to the on-target WS1-110A and OT1 sites.

FIG. 10 shows GUIDE-Seq for crIVS2-654T editing by LbCas12a-2×NLS in HEK293T cells by plasmid transient transfection. Unique read counts at 4 potential off-target sites (OT #), in addition to the on-target IVS2-654T site.

### DETAILED DESCRIPTION

Embodiments described herein are based in part to the discovery that allelic disruption of aberrant splice sites, one of the major classes of thalassemia mutations, is a robust approach to restore gene function. Specifically, the IVS1-110G>A mutation using Cas9 ribonucleoprotein (RNP) and the IVS2-654C>T mutation by Cas12a/Cpf1 RNP were targeted in primary CD34+ hematopoietic stem and progenitor cells (HSPCs) from β-thalassemia patients. Both of these nuclease complexes achieve high efficiency and penetrance of therapeutic edits. Erythroid progeny of edited patient HSPCs show reversal of aberrant splicing and restoration of β-globin expression.

Ribonucleoprotein (RNP) complexes, which comprises a polypeptide and RNA, are an effective means to introduce a gene editing tools to a cell or subject. Provided herein is a RNP complex comprising a DNA-targeting endonuclease Cas protein (e.g., a Cas enzyme) and a guide RNA comprising a sequence of SEQ ID NO: 1 or 3 that targets and hybridizes to a target sequence on a DNA molecule. In one embodiment, the sequence of the guide RNA is the sequence of SEQ ID NO: 1 or 3.

In one embodiment, the RNP complex comprises a Cas9 protein and a gRNA having a comprising or having a sequence of SEQ ID NO: 1. Such RNP complexes that comprise a Cas9 protein can be used to correct a IVS-1110G>A mutation that results in a cryptic splics site in the β-Globin gene.

In one embodiment, the RNP complex comprises a Cas12a protein (also known as Cpf1) and a gRNA having a comprising or having a sequence of SEQ ID NO: 3. Such RNP complexes that comprise a Cas12a protein can be used to correct a IVS2-654C>T mutation that results in a cryptic splice site in the β-Globin gene.

In one embodiment, RNP complexes described herein are be delivered to primary CD34+ hematopoietic stem and progenitor cells (HSPCs) from β-thalassemia patients to correct mutations described herein.

One aspect herein is an RNP complex comprising a Cas9 protein and a gRNA having a comprising or having a sequence of SEQ ID NO: 1.

Another aspect herein is an RNP complex comprising a Cas12a and a gRNA having a comprising or having a sequence of SEQ ID NO: 3.

Further provided herein are compositions comprising any of the RNP complexes described herein.

### CRISPR System

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a cell such that expression of the elements of the CRISPR system direct formation of a CRISPR complex at one or more target sites. For example, an NLS-Cas fusion enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In one embodiment, the RNP complex further comprises a crRNA/tracrRNA sequence. In one embodiment, the crRNA sequence is selected from SEQ ID NO: 1-4.

### CRISPR Enzyme

In one embodiment, the CRISPR enzyme is a Cas protein. Non-limiting examples of Cas proteins include Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c homologs thereof, or modified versions thereof. These enzymes are known; for example, the amino acid sequence of S. pyogenes Cas9 protein may be found in the SwissProt database under accession number Q99ZW2, and the amino acid sequence of S. pyogenes Cas12a protein may be found in the SwissProt database under accession number U2UMQ6. In some embodiments, the CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, the CRISPR enzyme directs cleavage of one or both strands within about 1, 2, 3, 4, 5, 6, 7, 8, 9, 10, 15, 20, 25, or more base pairs from the first or last nucleotide of a cryptic splice site.

In one embodiment, the CRISPR enzyme comprising at least one nuclear localization signal sequences (NLSs), e.g., at or near the amino-terminus, at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. Typically, an NLS consists of one or more short sequences of positively charged lysines or arginines exposed on the protein surface, but other types of NLS are known. Non-limiting examples of NLSs include an NLS sequence derived from: the NLS of the SV40 virus large T-antigen, having the amino acid sequence PKKKRKV (SEQ ID NO: 5); the NLS from nucleoplasmin (e.g. the nucleoplasmin bipartite NLS with the sequence KRPAATKKAGQAKKKK (SEQ ID NO: 6)); the c-myc NLS having the amino acid sequence PAAKRVKLD (SEQ ID NO: 7) or RQRRNELKRSP (SEQ ID NO: 8); the hRNPA1 M9 NLS having the sequence NQSSNFGPMKGGNFGGRSSGPYGGGGQYFAKPRNQGGY (SEQ ID NO: 9); the sequence RMRIZFKNKGKDTAELRRRRVEVSVELRKAKKDEQILKRRNV (SEQ ID NO: 10) of the IBB domain from importin-alpha; the sequences VSRKRPRP (SEQ ID NO: 11) and PPKKARED (SEQ ID NO: 12) of the myoma T protein; the sequence PQPKKKPL (SEQ ID NO: 13) of human p53; the sequence SALIKKKKKMAP (SEQ ID NO: 14) of mouse c-abl VI; the sequences DRLRR (SEQ ID NO: 15) and PKQKKRK (SEQ ID NO: 16) of the influenza virus NS1; the sequence RKLKKKIKKL (SEQ ID NO: 17) of the Hepatitis virus delta antigen; the sequence REKKKFLKRR (SEQ ID NO: 18) of the mouse M×1 protein; the sequence KRKGDEVDGVDEVAKKKSKK (SEQ ID NO: 19) of the human poly(ADP-ribose) polymerase; the sequence RKCLQAGMNLEARKTKK (SEQ ID NO: 20) of the steroid hormone receptors (human) glucocorticoid; the sequence GKRKLITSEEERSPAKRGRKS (SEQ ID NO: 21) of 53BP1; the sequence KRKRRP (SEQ ID NO. 22) of BRCA1; the sequence KRKGSPCDTLASSTEKRRRE (SEQ ID NO. 23) of SRC-1; and the sequence KRNFRSALNRKE (SEQ ID NO: 24) of IRF3.

In one embodiment, linkers are inserted in between at least one NLS sequence and the CRISPR enzyme sequence, and/or in between two NLS sequences. In one embodiment, at least 1, 2, 3, 4, 5, 6, 7, 8, 9, 10 or more linkers are included in the synthetic nucleic acid or polypeptides described herein. When more than one linker is used, the more than one linkers can be identical, or the more than one linkers can be different. Table 1 below presents nucleotide and protein seuqences for exemplary linkers.

TABLE 1
nucleotide and
protein sequences for exemplary linkers

| Protein | Corresponding nucleic |
|---|---|
| linker sequences | acid linker sequences |
| Gly-Gly-Ser-Gly | GGCGGTAGCGGC (SEQ ID NO: 29) |
| (SEQ ID NO: 25) | |
| (Gly-Gly-Ser-Gly)x3 | GGCGGTAGCGGCGGAGGCAGCGGTGGCG |
| (SEQ ID NO: 26) | GCAGCGGC (SEQ ID NO: 30) |
| (Gly-Gly-Ser-Gly)x5 | GGCGGTAGCGGCGGCGGTAGCGGCGGAG |
| (SEQ ID NO: 27) | GCAGCGGTGGCGGCAGCGGCGGCGGTAG |
| | CGGC (SEQ ID NO: 31) |
| TGGGPGGGAAAGSGS | ACCGGTGGTGGTCCCGGGGGTGGTGCGG |
| (SEQ ID NO: 28) | CCGCAGGCAGCGGAAGC |
| | (SEQ ID NO: 32) |
| SGGSSGGSSGSETPGTSES | Tctggaggatctagcggaggatcctctg |
| ATPESSGGSSGGS | gaagcgagacaccaggcacaagcgagtc |
| (SEQ ID NO: 31) | cgccacaccagagagctccggcggctcc |
| | tccggaggatcc (SEQ ID NO: 32) |

### Guide RNA

RNP complexes described herein further comprise a guide RNA that targets and hybridizes to a target sequence of a DNA molecule. As used herein, “hybridizes” or “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The sequence of the guide RNA (e.g., the sequence homologous to the target gene of interest) can be determined for the intended use. For example, to target the β-Globin gene, one would choose a guide RNA that targets and hybridize to the β-Globin gene sequence in a manner that effectively results in the desired alteration of the gene's expression. In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay known in the art. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything) has a single occurrence in the genome. A unique target sequence in a genome may include an S. pyogenes Cas9 target site of the form MMMMMMMMMNNNNNNNNNNNXGG where NNNNNNNNNNNXGG (N is A, G, T, or C; and X can be anything) has a single occurrence in the genome. Alternatively, the first 8 positions in the above mentioned unique sequences can be NNNNNNNN, for example, NNNNNNNNNNNNNNNNNNNNXGG.

As a further example, for the Lachnospiraceae bacterium ND2006 Cas12a or Acidaminococcus sp. (strain BV3L6) Cas12a, a unique target sequence in a genome may include a Cas12a target site of the form TTTVNNNNNNNNNNNNNNNMMMMMMM where TTTVNNNNNNNNNNNNNNNN (N is A, G, T, or C; V is A, G or C; and X can be anything) has a single occurrence in the genome. A unique target sequence in a genome may include an Lachnospiraceae bacterium ND2006 Cas12a or Acidaminococcus sp. (strain BV3L6) Cas12a target site of the form TTTVNNNNNNNNNNNNNNNNMMMMMMM where TTTVNNNNNNNNNNNNNNNN (N is A, G, T, or C; V is A, G or C; and X can be anything) has a single occurrence in the genome. Alternatively, the first 8 positions in the above mentioned unique sequences can be NNNNNNNN, for example, TTTVNNNNNNNNNNNNNNNNNNNNNNN.

In one embodiment, the gRNA of the invention targets and hybridizes at or near a cryptic splice site (i.e., a region of DNA having splice site consensus sequence resulting from a mutation of the endogenous sequence), for example, 0, 1, 2, 3, 4, 5, 6, 7, 8, 9, 10 or more base pairs up- or down-stream from the cryptic splice site. The RNP complex which comprises a gRNA that hybridizes at or near a cryptic splice site can alter the mutation resulting in the cryptic splice site to reverse the mutation and prevent aberrant splicing therefrom.

In one embodiment, the sequence of the gRNA comprises a sequence of SEQ ID NO: 1 or 3. In one embodiment, the sequence of the gRNA is the sequence of SEQ ID NO: 1 or 3. In one embodiment, the gRNA comprises, consists of, or consists essentially of a sequence having at least 90%, at least 91%, at least 92%, at least 93%, at least 94%, at least 95%, at least 96%, at least 97%, at least 98%, at least 99%, or more identical to SEQ ID NO: 1 or 3, and retains as least 50% of the function of SEQ ID NO: 1 or 3, e.g., targeting and hybridizing at or near a cryptic splice site.

In various embodiments, the sequence of the gRNA comprises a sequence selected from those listed in Table 2. In one embodiment, the sequence of the gRNA is the sequence selected from those listed in Table 2. In one embodiment, the gRNA comprises, consists of, or consists essentially of a sequence having at least 90%, at least 91%, at least 92%, at least 93%, at least 94%, at least 95%, at least 96%, at least 97%, at least 98%, at least 99%, or more identical to any sequence selected from those listed in Table 2, and retains as least 50% of the function of the sequence selected from those listed in Table 2, e.g., targeting and hybridizing at or near a cryptic splice site.

### Altering Gene Expression

Aspects described herein are directed to methods of altering the genetic sequence of a gene. For example, the RNP complexes or compositions thereof described herein can be used to correct, or reverse a genetic mutation in a given gene. For example, “altering refers to a substitution, deletion, or insertion of at least one nucleotide in the nucleotide sequence of a gene, or of at least one amino acid in the amino acid sequence of a gene product. Any standard technique for assessing the nucleotide or amino acid sequence of a gene or gene product, respectively, can be used to determine if the sequence is altered. For example, genome sequencing or PCR-based assays with primers specific to a particular sequence. It is specifically contemplated herein that any gene in the cell's genome can be altered using methods described herein.

In one embodiment, altering the expression of a gene is increasing the expression of the gene or gene product. In one embodiment, the expression of a gene or gene product is increased by at least 5% as compared to a reference level. In one embodiment, the expression of a gene or gene product is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more, or at least about a 2-fold, or at least about a 3-fold, or at least about a 4-fold, or at least about a 5-fold or at least about a 10-fold increase, or any increase between 2-fold and 10-fold or greater as compared to a reference level. As used herein, “reference level” refers to the level of the gene or gene product in an otherwise identical sample that is not contacted with an RNP complex, edited cell, or composition thereof described herein. In the context of a marker or symptom, an “increase” is a statistically significant increase in such level. Any method known in the art can be used to measure an increase in expression a gene or gene product, e. g. PCR-based assays or Western Blot analysis to measure mRNA or protein levels, respectively.

In one embodiment, altering the expression of a gene is decreasing the expression of the gene or gene product. In one embodiment, the expression of a gene or gene product is decreased by at least 5% as compared to a reference level. In one embodiment, the expression of a gene or gene product is decreased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more as compared to a reference level. As used herein, “reference level” refers to the level of the gene or gene product in an otherwise identical sample that is not contacted with an RNP complex, edited cell, or composition thereof described herein. In the context of a marker or symptom, an “decrease” is a statistically significant decrease in such level. Any method known in the art can be used to measure a decrease in a gene or gene product, e. g. PCR-based assays or Western Blot analysis to measure mRNA or protein levels, respectively. Where applicable, a decrease can be preferably down to a level accepted as within the range of normal for an individual without a given disorder.

As used herein, the term “genome editing” and “gene editing” refers to a reverse genetics method using artificially engineered nucleases to cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point.

One aspect provided herein for altering the expression of a gene product comprises introducing into a cell any of the RNP complexes or compositions thereof described herein.

RNP complexes or compositions thereof described herein can be used to promote proper intron splicing, e.g., in a gene having a mutation resulting in a cryptic splice site. Thus, the RNP complexes or compositions thereof described herein can be used to correct, or reverse a mutation resulting in a cryptic splice site. In one embodiment, the gene having a cryptic splice site is β-Globin. In one embodiment, the genetic mutation resulting in a cryptic splice site is IVS1-110G>A or IVS2-654C>T. In various embodiment, the gene having a cryptic splice site is selected from those genes listed in Table 2.

In one embodiment, the RNP complex or composition thereof, or composition of edited cells described herein is used in an ex vivo method of producing a progenitor cell or a population of progenitor cell wherein the cells or the differentiated progeny thereof have an altered genetic sequence.

In one embodiment, the RNP complex or composition thereof, or composition of edited cells described herein is used in an ex vivo method of producing a progenitor cell or a population of progenitor cell wherein the cells or the differentiated progeny thereof have corrected a IVS1-110G>A or IVS2-654C>T mutation.

In one embodiment, the RNP complex or composition thereof, or composition of edited cells described herein is used in an ex vivo method of producing a progenitor cell or a population of progenitor cell wherein the cells or the differentiated progeny thereof have at least one genetic modification in the β-Globin gene.

In one embodiment, the RNP complex or composition thereof, or composition of edited cells described herein is used in an ex vivo method of producing an isolated genetic engineered human cell or a population of genetic engineered human cells having an altered genetic sequence.

In one embodiment, the RNP complex or composition thereof, or composition of edited cells described herein is used in an ex vivo method of producing an isolated genetic engineered human cell or a population of genetic engineered human cells which have corrected a IVS1-110G>A or IVS2-654C>T mutation.

In one embodiment, the RNP complex or composition thereof, or composition of edited cells described herein is used in an ex vivo method of producing an isolated genetic engineered human cell or a population of genetic engineered human cells having at least one genetic modification in the β-Globin gene.

Further provided herein is a method for correcting an isolated progenitor cell or a population of isolated progenitor cells having a IVS1-110G>A or IVS2-654C>T mutation in the β-Globin gene comprising contacting an isolated progenitor cell with an effective amount of any RNP complex or composition thereof, or composition of edited cells described herein, whereby the contacted cells or the differentiated progeny cells therefrom have corrected the IVS1-110G>A or IVS2-654C>T mutation in the β-Globin gene.

In one embodiment, the methods and compositions described herein are used for altering the expression of adult hemoglobin. In another embodiment, the methods described herein are used for increasing the expression of adult hemoglobin. As used herein the term “increasing the adult hemoglobin levels” in a cell indicates that adult hemoglobin is at least 5% higher in populations treated with any agent (e.g., RNP complex, edited cell, or composition thereof), than in a comparable, control population, wherein no agent is present. It is preferred that the percentage of adult hemoglobin expression in a population treated with such NLS-CRISPR enzyme described herein is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a control treated population of comparable size and culture conditions. The term “control treated population” is used herein to describe an otherwise identical population of cells (e.g., that has been treated with identical media, viral induction, nucleic acid sequences, temperature, confluency, flask size, pH, etc.) that is not treated with any of the agents described herein. In one embodiment, any method known in the art can be used to measure an increase in adult hemoglobin expression, e. g. Western Blot analysis of adult β-globin protein and quantifying mRNA of adult β-globin.

### Engineered Cells

In one embodiment, the RNP complex, or a composition thereof described herein can be used to engineer a cell that has an altered gene expression as compared to a wild-type cell. In another embodiment, the methods described herein can be used to engineer a cell that has an altered gene expression as compared to a wild-type cell. For example, a HSC can be engineered to have altered β-Globin gene, such that a mutation resulting in a cryptic splice site is corrected in the β-Globin gene using methods described herein. In one embodiment, the engineered cell is a HSC or a cell derived therefrom. In one embodiment, the engineered cell is a HSC that can be administered to a subject in need thereof. In one embodiment, the engineered cell can be an isolated cell, or can be comprised in an isolated population.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched. In some embodiments, the isolated population is an isolated population of engineered human hematopoietic progenitor cells, e.g., a substantially pure population of engineered human hematopoietic progenitor cells as compared to a heterogeneous population of cells comprising engineered human hematopoietic progenitor cells and cells from which the human hematopoietic progenitor cells were derived.

Isolated populations of cells useful as a therapeutic are often desired to be substantially pure. The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of, for example, engineered hematopoietic progenitor cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not engineered hematopoietic progenitor cells as defined by the terms herein.

In one embodiment, the engineered cell can be comprised in a composition. In another embodiment, the engineered cell can be comprised in a pharmaceutical composition. A composition of cell described herein can further comprise a pharmaceutically acceptable carrier. It is desired that any pharmaceutically acceptable carrier used is beneficial in promoting the health and/or growth of the cells and does not result in an adverse effect or negatively impact the cells comprised in the composition. For example, a carrier that results in cell death or alters the physiological properties (e.g., size, shape, pH, etc.) would not be desired.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

In one embodiment, the population of edited cells, e.g., edited hematopoietic progenitor or stem cells, is cryopreserved and stored or reintroduced into the mammal. In another embodiment, the cryopreserved population of edited hematopoietic progenitor or stem cells is thawed and then reintroduced into the mammal. In further embodiment of this method, the method comprises administering to a subject chemotherapy and/or radiation therapy to remove or reduced the endogenous hematopoietic progenitor or stem cells prior to reintroducing thawed cells into the subject. In certain embodiments, the methods further comprises selecting a subject in need of expression of altered β-Globin, e.g., a subject having a mutation resulting in a cryptic splice site as described herein.

Hematopoietic progenitor or stem cells can be substituted with an iPSCs described herein in all methods and compositions described herein. In various embodiments, the hematopoietic progenitor or stem cells or iPSCs are autologous to the mammal, meaning the cells are derived from the same mammal. Alternatively, the hematopoietic progenitor or stem cells or iPSCs are non-autologous to the mammal, meaning the cells are not derived from the same mammal, but another mammal of the same species. For example, the mammal is a human.

In one embodiment, the cells of any compositions described herein are autologous to the subject who is the recipient of the cells in a transplantation procedure, i.e., the cells of the composition are derived or harvested from the subject prior to any described modification. In one embodiment of this method, the method comprises administering to a subject chemotherapy and/or radiation therapy to remove or reduced the endogenous hematopoietic progenitor or stem cells aftern harvesting the cells, and prior to reintroducing the cells into the subject.

In one embodiment, the cells of any compositions described are non-autologous to the subject who is the recipient of the cells in a transplantation procedure, i.e., the cells of the composition are not derived or harvested from the subject prior to any described modification.

In one embodiment, the cells of any compositions described are at the minimum HLA type matched with to the subject who is the recipient of the cells in a transplantation procedure.

In one embodiment, a cell is any cell produced using methods described herein. In one embodiment, a composition comprises any cell produced using methods described herein.

The genetically modified cells may be administered as part of a bone marrow or cord blood transplant in an individual that has or has not undergone bone marrow ablative therapy. In one embodiment, genetically modified cells contemplated herein are administered in a bone marrow transplant to an individual that has undergone chemoablative or radioablative bone marrow therapy.

In one embodiment, a dose of genetically modified cells is delivered to a subject intravenously. In one embodiment, genetically modified hematopoietic cells are intravenously administered to a subject.

In particular embodiments, patients receive a dose of genetically modified cells, e.g., hematopoietic stem cells, of about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single intravenous dose. In certain embodiments, patients receive a dose of genetically modified cells, e.g., hematopoietic stem cells described herein or genetic engineered cells described herein or progeny thereof, of at least 1×10⁵ cells/kg, at least 5×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 5×10⁷ cells/kg, at least 1×10⁸ cells/kg, or more in one single intravenous dose.

In an additional embodiment, patients receive a dose of genetically modified cells, e.g., hematopoietic stem cells, of about 1×10⁵ cells/kg to about 1×10⁸ cells/kg, about 1×10⁶ cells/kg to about 1×10⁸ cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In various embodiments, the methods described here provide more robust and safe gene therapy than existing methods and comprise administering a population or dose of cells comprising about 5% transduced/ genetically modified cells, about 10% transduced/genetically modified cells, about 15% transduced/genetically modified cells, about 20% transduce/genetically modified d cells, about 25% transduced/genetically modified cells, about 30% transduced/genetically modified cells, about 35% transduced/genetically modified cells, about 40% transduced/genetically modified cells, about 45% transduced/genetically modified cells, or about 50% transduce/genetically modified cells, to a subject.

In one embodiment, the invention provides genetically modified cells, such as a stem cell, e.g., hematopoietic stem cell, with the potential to expand or increase a population of erythroid cells. Hematopoietic stem cells are the origin of erythroid cells and thus, are preferred.

In one embodiment, the hematopoietic stem cell or hematopoietic progenitor cell is collected from peripheral blood, cord blood, chorionic villi, amniotic fluid, placental blood, or bone marrow.

In one embodiment, the contacted hematopoietic stem cells described herein or genetic engineered cells described herein or the the progeny cells thereof are treated ex vivo with prostaglandin E2 and/or antioxidant N-acetyl-L-cysteine (NAC) to promote subsequent engraftment in a recipient subject.

In one embodiment, the method further comprises obtaining a sample or a population of embryonic stem cells, somatic stem cells, progenitor cells, bone marrow cells, hematopoietic stem cells, or hematopoietic progenitor cells from the subject.

In one embodiment, the embryonic stem cells, somatic stem cells, progenitor cells, bone marrow cells, hematopoietic stem cells, hematopoietic progenitor cells are isolated from the host subject, transfected, cultured (optional), and transplanted back into the same host, i. e. an autologous cell transplant. In another embodiment, the embryonic stem cells, somatic stem cells, progenitor cells, bone marrow cells, hematopoietic stem cells, or hematopoietic progenitor cells are isolated from a donor who is an HLA-type match with a host (recipient) who is diagnosed with or at risk of developing a hemoglobinopathy. Donor-recipient antigen type-matching is well known in the art. The HLA-types include HLA-A, HLA-C, and HLA-D. These represent the minimum number of cell surface antigen matching required for transplantation. That is the transfected cells are transplanted into a different host, i.e., allogeneic to the recipient host subject. The donor's or subject's embryonic stem cells, somatic stem cells, progenitor cells, bone marrow cells, hematopoietic stem cells, or hematopoietic progenitor cells can be contacted (electroporated) with a nucleic acid molecule described herein, the contacted cells are culture expanded, and then transplanted into the host subject. In one embodiment, the transplanted cells engraft in the host subject. The transfected cells can also be cryopreserved after transfected and stored, or cryopreserved after cell expansion and stored.

In one aspect of any method, the embryonic stem cell, somatic stem cell, progenitor cell, bone marrow cell, hematopoietic stem cell, or hematopoietic progenitor cell is autologous or allogeneic to the subject.

In a further embodiment of any methods described herein, the recipient subject is treated with chemotherapy and/or radiation prior to implantation of the contacted or transfected cells (i.e., the contacted hematopoietic stem cells described herein or genetic engineered cells described herein or the the progeny cells thereof). The chemotherapy and/or radiation is to reduce endogenous stem cells to facilitate engraftment of the implanted cells.

### Hemoglobinopathies

Provided herein is a method of treating a disease associated with IVS1-110G>A or IVS2-654C>T mutation in the β-Globin gene comprising administering to a subject in need thereof any of the RNP complexes or compositions thereof, or any of the genetically edited progenitor cells or compositions thereof described herein. In one embodiment, the disease is thalassemia or β-thalassemia.

Fetal hemoglobin (HbF) is a tetramer of two adult α-globin polypeptides and two fetal β-like γ-globin polypeptides. During gestation, the duplicated γ-globin genes constitute the predominant genes transcribed from the β-globin locus. Following birth, γ-globin becomes progressively replaced by adult β-globin, a process referred to as the “fetal switch” (3). The molecular mechanisms underlying this switch have remained largely undefined and have been a subject of intense research. The developmental switch from production of predominantly fetal hemoglobin or HbF (α2γ2) to production of adult hemoglobin or HbA (α2β2) begins at about 28 to 34 weeks of gestation and continues shortly after birth at which point HbA becomes predominant. This switch results primarily from decreased transcription of the gamma-globin genes and increased transcription of beta-globin genes. On average, the blood of a normal adult contains only about 2% HbF, though residual HbF levels have a variance of over 20 fold in healthy adults (Atweh, Semin. Hematol. 38(4):367-73 (2001)).

Hemoglobinopathies encompass a number of anemias of genetic origin in which there is a decreased production and/or increased destruction (hemolysis) of red blood cells (RBCs). These disorders also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders specifically associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickled) hemoglobin (HbS). HbS RBCs are more fragile than normal RBCs and undergo hemolysis more readily, leading eventually to anemia (Atweh, Semin. Hematol. 38(4):367-73 (2001)). Moreover, the presence of a BCL11A genetic variant, HBS1L-MYB variation, ameliorates the clinical severity in beta-thalassemia. This variant has been shown to be associated with HbF levels. It has been shown that there is an odds ratio of 5 for having a less severe form of beta-thalassemia with the high-HbF variant (Galanello S. et al., 2009, Blood, in press).

As used herein, treating or reducing a risk of developing a hemoglobinopathy in a subject means to ameliorate at least one symptom of hemoglobinopathy. In one aspect, the invention features methods of treating, e.g., reducing severity or progression of, a hemoglobinopathy in a subject. In another aspect, the methods can also be used to reduce a risk of developing a hemoglobinopathy in a subject, delaying the onset of symptoms of a hemoglobinopathy in a subject, or increasing the longevity of a subject having a hemoglobinopathy. In one aspect, the methods can include selecting a subject on the basis that they have, or are at risk of developing, a hemoglobinopathy, but do not yet have a hemoglobinopathy, or a subject with an underlying hemoglobinopathy. Selection of a subject can include detecting symptoms of a hemoglobinopathy, a blood test, genetic testing, or clinical recordings. If the results of the test(s) indicate that the subject has a hemoglobinopathy, the methods also include administering the compositions described herein, thereby treating, or reducing the risk of developing, a hemoglobinopathy in the subject. For example, a subject who is diagnosis of β-thalassemia with genotype β⁺β0 thalassemia.

As used herein, the term “hemoglobinopathy” refers to a condition involving the presence of an abnormal hemoglobin molecule in the blood. Examples of hemoglobinopathies include, but are not limited to, SCD and THAL. Also included are hemoglobinopathies in which a combination of abnormal hemoglobins is present in the blood (e.g., sickle cell/Hb-C disease). An exemplary example of such a disease includes, but is not limited to, SCD and THAL. SCD and THAL and their symptoms are well-known in the art and are described in further detail below. Subjects can be diagnosed as having a hemoglobinopathy by a health care provider, medical caregiver, physician, nurse, family member, or acquaintance, who recognizes, appreciates, acknowledges, determines, concludes, opines, or decides that the subject has a hemoglobinopathy.

The term “SCD” is defined herein to include any symptomatic anemic condition which results from sickling of red blood cells. Manifestations of SCD include: anemia; pain; and/or organ dysfunction, such as renal failure, retinopathy, acute-chest syndrome, ischemia, priapism, and stroke. As used herein the term “SCD” refers to a variety of clinical problems attendant upon SCD, especially in those subjects who are homozygotes for the sickle cell substitution in HbS. Among the constitutional manifestations referred to herein by use of the term of SCD are delay of growth and development, an increased tendency to develop serious infections, particularly due to pneumococcus, marked impairment of splenic function, preventing effective clearance of circulating bacteria, with recurrent infarcts and eventual destruction of splenic tissue. Also included in the term “SCD” are acute episodes of musculoskeletal pain, which affect primarily the lumbar spine, abdomen, and femoral shaft, and which are similar in mechanism and in severity. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia, and dehydration, all of which potentiate intracellular polymerization of HbS (J. H. Jandl, Blood: Textbook of Hematology, 2nd Ed., Little, Brown and Company, Boston, 1996, pages 544-545).

As used herein, “THAL” refers to a hereditary disorder characterized by defective production of hemoglobin. In one embodiment, the term encompasses hereditary anemias that occur due to mutations affecting the synthesis of hemoglobins. In other embodiments, the term includes any symptomatic anemia resulting from thalassemic conditions such as severe or β-thalassemia, thalassemia major, thalassemia intermedia, α-thalassemias such as hemoglobin H disease. β-thalassemias are caused by a mutation in the β-globin chain, and can occur in a major or minor form. In the major form of β-thalassemia, children are normal at birth, but develop anemia during the first year of life. The mild form of β-thalassemia produces small red blood cells. Alpha-thalassemias are caused by deletion of a gene or genes from the globin chain.

By the phrase “risk of developing disease” is meant the relative probability that a subject will develop a hemoglobinopathy in the future as compared to a control subject or population (e.g., a healthy subject or population). For example, an individual carrying the genetic mutation associated with SCD, an A to T mutation of the β-globin gene, and whether the individual in heterozygous or homozygous for that mutation increases that individual's risk.

### Hematopoietic Progenitor Cells

In one embodiment, the hematopoietic progenitor cell is contacted, e.g., with a RNP complex or composition described herein, ex vivo or in vitro. In a specific embodiment, the cell being contacted is a cell of the erythroid lineage. In one embodiment, the cell composition comprises cells having increased, proper splicing of the β-Globin gene.

In one embodiment, the cell is a quiescent cell. As used herein, “quiescent cell” refers to a cell in a reversible state in which it does not divide but retains the ability to re-enter cell proliferation. Exemplary quiescent cells include, but are not limited to, a hematopoietic stem cell, a muscle stem cell, a neural stem cell, an intestinal stem cell, a skin stem cell or epidermal stem cell, a mesenchymal stem cell, a resting T cell, a memory T cell, a neuron, a neuronal stem cell, a myotube or skeletal myoblast or satellite cell, and a hepatocyte.

“Hematopoietic progenitor cell” as the term is used herein, refers to cells of a stem cell lineage that give rise to all the blood cell types including the myeloid (monocytes and macrophages, neutrophils, basophils, eosinophils, erythrocytes, megakaryocytes/platelets, dendritic cells), and the lymphoid lineages (T-cells, B-cells, NK-cells). A “cell of the erythroid lineage” indicates that the cell being contacted is a cell that undergoes erythropoiesis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage”, as the term is used herein, comprise hematopoietic progenitor cells, rubriblasts, prorubricytes, erythroblasts, metarubricytes, reticulocytes, and erythrocytes.

In some embodiment, the hematopoietic progenitor cell has at least one of the cell surface marker characteristic of hematopoietic progenitor cells: CD34+, CD59+, Thy1/CD90+, CD38lo/−, and C-kit/CD117+. Preferably, the hematopoietic progenitor cells have several of these markers. One skilled in the art can assess if a cell, e.g., a hematopoietic progenitor cell, comprises as least one marker described herein above using standard techniques, for example, FACS sorting.

In some embodiments, the hematopoietic progenitor cells of the erythroid lineage have the cell surface marker characteristic of the erythroid lineage: CD71 and Ter119. One skilled in the art can assess if a cell, e.g., of the erythroid lineage, comprises as least one marker described herein above using standard techniques, for example, FACS sorting.

Stem cells, such as hematopoietic progenitor cells, are capable of proliferation and giving rise to more progenitor cells having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a hematopoietic progenitor cell), which in turn can differentiate into other types of precursor cells further down the pathway (such as an erythrocyte precursor), and then to an end-stage differentiated cell, such as an erythrocyte, which plays a characteristic role in a certain tissue type, and may or may not retain the capacity to proliferate further.

### Induced Pluripotent Stem Cells

In some embodiments, the genetic engineered human cells described herein are derived from isolated pluripotent stem cells. An advantage of using iPSCs is that the cells can be derived from the same subject to which the progenitor cells are to be administered. That is, a somatic cell can be obtained from a subject, reprogrammed to an induced pluripotent stem cell, and then re-differentiated into a hematopoietic progenitor cell to be administered to the subject (e.g., autologous cells). Since the progenitors are essentially derived from an autologous source, the risk of engraftment rejection or allergic responses is reduced compared to the use of cells from another subject or group of subjects. In some embodiments, the hematopoietic progenitors are derived from non-autologous sources. In addition, the use of iPSCs negates the need for cells obtained from an embryonic source. Thus, in one embodiment, the stem cells used in the disclosed methods are not embryonic stem cells.

Although differentiation is generally irreversible under physiological contexts, several methods have been recently developed to reprogram somatic cells to induced pluripotent stem cells. Exemplary methods are known to those of skill in the art and are described briefly herein below.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods described herein can also be of use for such purposes, in some embodiments.

The specific approach or method used to generate pluripotent stem cells from somatic cells (broadly referred to as “reprogramming”) is not critical to the claimed invention. Thus, any method that re-programs a somatic cell to the pluripotent phenotype would be appropriate for use in the methods described herein.

Reprogramming methodologies for generating pluripotent cells using defined combinations of transcription factors have been described induced pluripotent stem cells. Yamanaka and Takahashi converted mouse somatic cells to ES cell-like cells with expanded developmental potential by the direct transduction of Oct4, Sox2, Klf4, and c-Myc (Takahashi and Yamanaka, 2006). iPSCs resemble ES cells as they restore the pluripotency-associated transcriptional circuitry and muc of the epigenetic landscape. In addition, mouse iPSCs satisfy all the standard assays for pluripotency: specifically, in vitro differentiation into cell types of the three germ layers, teratoma formation, contribution to chimeras, germline transmission (Maherali and Hochedlinger, 2008), and tetraploid complementation (Woltjen et al., 2009).

Subsequent studies have shown that human iPS cells can be obtained using similar transduction methods (Lowry et al., 2008; Park et al., 2008; Takahashi et al., 2007; Yu et al., 2007b), and the transcription factor trio, OCT4, SOX2, and NANOG, has been established as the core set of transcription factors that govern pluripotency (Jaenisch and Young, 2008). The production of iPS cells can be achieved by the introduction of nucleic acid sequences encoding stem cell-associated genes into an adult, somatic cell, historically using viral vectors.

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 2010 Nov. 5; 7(5):618-30). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise introducing one or more of each of Oct 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (-)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

### Somatic Cells for Reprogramming

Somatic cells, as that term is used herein, refer to any cells forming the body of an organism, excluding germline cells. Every cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

When reprogrammed cells are used for generation of hematopoietic progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2;β-catenin, and Bmi1. Such cells can also be characterized by the down-regulation of markers characteristic of the somatic cell from which the induced pluripotent stem cell is derived.

### Pharmaceutically Acceptable Carriers

The methods of administering human hematopoietic progenitor cells or genetic engineered cells described herein or their progeny to a subject as described herein involve the use of therapeutic compositions comprising said hematopoietic progenitor cells. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired.

In general, the hematopoietic progenitor cells described herein or genetic engineered cells described herein or their progeny are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the hematopoietic progenitor cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

In some embodiments, the compositions of isolated genetic engineered cells described further comprises a pharmaceutically acceptable carrier. In one embodiment, the pharmaceutically acceptable carrier does not include tissue or cell culture media.

In some embodiments, the compositions of RNP complexes described further comprises a pharmaceutically acceptable carrier. In one embodiment, the pharmaceutically acceptable carrier does not include tissue or cell culture media.

### Administration & Efficacy

As used herein, the terms “administering,” “introducing” and “transplanting” are used interchangeably in the context of the placement of cells, e.g. hematopoietic progenitor cells, as described herein into a subject, by a method or route which results in at least partial localization of the introduced cells at a desired site, such as a site of injury or repair, such that a desired effect(s) is produced. The cells e.g. hematopoietic progenitor cells, or their differentiated progeny can be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. For example, in some embodiments of the aspects described herein, an effective amount of hematopoietic progenitor cells or engineered cells with proper β-Globin splicing is administered via a systemic route of administration, such as an intraperitoneal or intravenous route.

When provided prophylactically, hematopoietic progenitor cells or engineered cells with proper β-Globin splicing described herein can be administered to a subject in advance of any symptom of a hemoglobinopathy, e.g., prior to the switch from fetal γ-globin to predominantly β-globin. Accordingly, the prophylactic administration of a hematopoietic progenitor cell population serves to prevent a hemoglobinopathy, as disclosed herein.

When provided therapeutically, hematopoietic progenitor cells are provided at (or after) the onset of a symptom or indication of a hemoglobinopathy, e.g., upon the onset of β-thalassemia.

In some embodiments of the aspects described herein, the hematopoietic progenitor cell population or engineered cells with proper β-Globin splicing being administered according to the methods described herein comprises allogeneic hematopoietic progenitor cells obtained from one or more donors. As used herein, “allogeneic” refers to a hematopoietic progenitor cell or biological samples comprising hematopoietic progenitor cells obtained from one or more different donors of the same species, where the genes at one or more loci are not identical. For example, a hematopoietic progenitor cell population or engineered cells with proper β-Globin splicing being administered to a subject can be derived from umbilical cord blood obtained from one more unrelated donor subjects, or from one or more non-identical siblings. In some embodiments, syngeneic hematopoietic progenitor cell populations can be used, such as those obtained from genetically identical animals, or from identical twins. In other embodiments of this aspect, the hematopoietic progenitor cells are autologous cells; that is, the hematopoietic progenitor cells are obtained or isolated from a subject and administered to the same subject, i.e., the donor and recipient are the same.

For use in the various aspects described herein, an effective amount of hematopoietic progenitor cells or engineered cells with proper β-Globin splicing comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ hematopoietic progenitor cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The hematopoietic progenitor cells or engineered cells with proper β-Globin splicing can be derived from one or more donors, or can be obtained from an autologous source. In some embodiments of the aspects described herein, the hematopoietic progenitor cells are expanded in culture prior to administration to a subject in need thereof.

In one embodiment, the term “effective amount” as used herein refers to the amount of an agent described herein (e.g., an RNP complex, a population of human hematopoietic progenitor cells or their progeny, or composition thereof) needed to alleviate at least one or more symptom of a hemoglobinopathy, and relates to a sufficient amount of a composition to provide the desired effect, e.g., treat a subject having a hemoglobinopathy. The term “therapeutically effective amount” therefore refers to an amount of an agent described herein that is sufficient to promote a particular effect when administered to a typical subject, such as one who has or is at risk for a hemoglobinopathy. An effective amount as used herein would also include an amount sufficient to prevent or delay the development of a symptom of the disease, alter the course of a symptom disease (for example but not limited to, slow the progression of a symptom of the disease), or reverse a symptom of the disease. It is understood that for any given case, an appropriate “effective amount” can be determined by one of ordinary skill in the art using routine experimentation.

As used herein, “administered” refers to the delivery an agent described herein (e.g., an RNP complex, a population of human hematopoietic progenitor cells or their progeny, or composition thereof) into a subject by a method or route which results in at least partial localization of the agent at a desired site. An agent can be administered by any appropriate route which results in effective treatment in the subject, i.e. administration results in delivery to a desired location in the subject where at least a portion of the composition delivered, i.e. a composition of at least 1×10⁴ cells are delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. For the delivery of cells or compositions thereof, administration by injection or infusion is generally preferred.

In one embodiment, the cells as described herein are administered systemically. The phrases “systemic administration,” “administered systemically”, “peripheral administration” and “administered peripherally” as used herein refer to the administration of an agent described herein (e.g., an RNP complex, a population of human hematopoietic progenitor cells or their progeny, or composition thereof) other than directly into a target site, tissue, or organ, such that it enters, instead, the subject's circulatory system and, thus, is subject to metabolism and other like processes.

The efficacy of a treatment comprising a composition as described herein for the treatment of a hemoglobinopathy can be determined by the skilled clinician. However, a treatment is considered “effective treatment,” as the term is used herein, if any one or all of the signs or symptoms of, as but one example, levels of proper β-Globin splicing are altered in a beneficial manner, other clinically accepted symptoms or markers of disease are improved or ameliorated, e.g., by at least 10% following treatment with an RNP. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of sepsis; or (2) relieving the disease, e.g., causing regression of symptoms; and (3) preventing or reducing the likelihood of the development of infection or sepsis.

The treatment according to the present invention ameliorates one or more symptoms associated with a β-globin disorder by increasing the amount of proper β-Globin splicing in the individual. Symptoms typically associated with a hemoglobinopathy, include for example, anemia, tissue hypoxia, organ dysfunction, abnormal hematocrit values, ineffective erythropoiesis, abnormal reticulocyte (erythrocyte) count, abnormal iron load, the presence of ring sideroblasts, splenomegaly, hepatomegaly, impaired peripheral blood flow, dyspnea, increased hemolysis, jaundice, anemic pain crises, acute chest syndrome, splenic sequestration, priapism, stroke, hand-foot syndrome, and pain such as angina pectoris.

In one embodiment, the hematopoietic progenitor cell is contacted ex vivo or in vitro with a DNA targeting endonuclease, and the cell or its progeny is administered to the mammal (e.g., human). In a further embodiment, the hematopoietic progenitor cell is a cell of the erythroid lineage. In one embodiment, a composition comprising a hematopoietic progenitor cell that was previously contacted with a DNA-targeting endonuclease and a pharmaceutically acceptable carrier and is administered to a mammal.

In one embodiment, any method known in the art can be used to measure an increase in adult hemoglobin expression, e.g., PCR-based assays and Western Blot analysis to assess mRNA and protein levels of adult β-globin, respectively.

In one embodiment, the hematopoietic progenitor cell is contacted with a RNP complex described herein in vitro, or ex vivo. In one embodiment, the cell is of human origin (e.g., an autologous or heterologous cell). In one embodiment, the composition causes an increase in fetal hemoglobin expression in the host it is delivered, for example a human subject, or a cell.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Furthermore, the disclosure described herein does not concern the destruction of a human embryo.

This invention is further illustrated by the following example which should not be construed as limiting. The contents of all references cited throughout this application, as well as the figures and table are incorporated herein by reference.

Some embodiments of the invention described herein can be defined according to any of the following numbered paragraphs:
1) A method of treating a disease caused by or associated with a mutation resulting in an aberrant splice site in a gene in a subject in need thereof, the method comprising:
contacting a cell obtained from the subject with a DNA editing enzyme configured to correct, disrupt, or delete the mutation; and
administering the cell resulting from step a to the subject.
2) A method of treating a disease caused by or associated with a mutation resulting in an aberrant splice site in a gene in a subject in need thereof, the method comprising:
contacting a cell in a subject with a DNA editing enzyme configured to correct, disrupt, or delete the mutation.
3) The method of any preceding paragraph, wherein the cell is a stem or progenitor cell.
4) The method of any preceding paragraph, wherein the cell is a hematopoietic stem and progenitor cell (HPSC) or hematopoietic stem cell (HSC).
5) The method of any preceding paragraph, wherein the DNA editing enzyme is a CRISPR enzyme, a base editor, or nuclease.
6) The method of any preceding paragraph, wherein the CRISPR enzyme is Cas9, SpCas9, Cas12a, or LbCas12a.
7) The method of any preceding paragraph, wherein the CRISPR is provided with a crRNA having the sequence of any of SEQ ID NOs: 1-4.
8) The method of any preceding paragraph, wherein the DNA editing enzyme is provided in a RNP.
9) The method of any preceding paragraph, wherein the cell is further contacted with a template nucleic acid that comprises a sequence of the gene in which the mutation is corrected, disrupted, or deleted.
10) The method of any preceding paragraph, wherein the gene is β-globin.
11) The method of any preceding paragraph, wherein the mutation is IVS1-110G>A or IVS2-654C>T.
12) The method of any preceding paragraph, wherein the mutation is a mutation selected from Table 2.
13) The method of any preceding paragraph, wherein the disease is thalassemia or β-thalassemia.

Some embodiments of the invention described herein can be further defined according to any of the following additional numbered paragraphs:
1) A ribonucleoprotein (RNP) complex comprising a DNA-targeting endonuclease Cas (CRISPR-associated) protein and a guide RNA comprising the sequence of SEQ ID NO: 1 or 3 that targets and hybridizes to a target sequence on a DNA molecule.
2) The RNP complex of any of the preceding paragraphs, wherein the CRISPR enzyme is a type II CRISPR system enzyme.
3) The RNP complex of any of the preceding paragraphs, wherein the CRISPR enzyme is a Cas enzyme.
4) The RNP complex of any of the preceding paragraphs, wherein the Cas protein is selected from the group consisting of: Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Cs1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.
5) The RNP complex of any of the preceding paragraphs, wherein the Cas protein is Cas9 or Cas12a.
6) T The RNP complex of any of the preceding paragraphs for use in altering the genetic sequence of a gene.
7) The RNP complex of any of the preceding paragraphs, wherein altering is a nucleotide deletion, insertion or substitution of the genetic sequence.
8) The RNP complex of any of the preceding paragraphs, wherein altering promotes proper intron splicing of a gene.
9) The RNP complex of any of the preceding paragraphs, wherein altering is correcting a genetic mutation in a gene.
10) The RNP complex of any of the preceding paragraphs, wherein the gene is β-Globin.
11) The RNP complex of any of the preceding paragraphs, wherein the genetic mutation is IVS1-110G>A or IVS2-654C>T.
12) The RNP complex of any of the preceding paragraphs, wherein the genetic mutation is selected from those listed in Table 2.
13) The RNP complex of any of the preceding paragraphs, wherein the guide RNA comprises a sequence selected from those listed in Table 2.
14) The RNP complex of any of the preceding paragraphs, further comprising a crRNA/tracrRNA sequence.
15) The RNP complex of any of the preceding paragraphs for use in an ex vivo method of producing a progenitor cell or a population of progenitor cell wherein the cells or the differentiated progeny thereof have an altered genetic sequence.
16) The RNP complex of any of the preceding paragraphs for use in an ex vivo method of producing a progenitor cell or a population of progenitor cell wherein the cells or the differentiated progeny thereof have corrected a IVS1-110G>A or IVS2-654C>T mutation.
17) The RNP complex of any of the preceding paragraphs for use in an ex vivo method of producing a progenitor cell or a population of progenitor cell wherein the cells or the differentiated progeny thereof have at least one genetic modification in the β-Globin gene.
18) The RNP complex of any of the preceding paragraphs for use in an ex vivo method of producing an isolated genetic engineered human cell or a population of genetic engineered human cells having an altered genetic sequence.
19) The RNP complex of any of the preceding paragraphs for use in an ex vivo method of producing an isolated genetic engineered human cell or a population of genetic engineered human cells which have corrected a IVS1-110G>A or IVS2-654C>T mutation.
20) The RNP complex of any of the preceding paragraphs for use in an ex vivo method of producing an isolated genetic engineered human cell or a population of genetic engineered human cells having at least one genetic modification in the β-Globin gene.
21) The RNP complex of any of the preceding paragraphs, wherein the cell is a hematopoietic progenitor cell or a hematopoietic stem cell.
22) The RNP complex of any of the preceding paragraphs, wherein the hematopoietic progenitor is a cell of the erythroid lineage.
23) The RNP complex of any of the preceding paragraphs, wherein the isolated human cell is an induced pluripotent stem cell.
24) The RNP complex of any of the preceding paragraphs, wherein IVS1-110G>A or IVS2-654C>T mutation is present in the β-Globin gene
25) A composition comprising the RNP complex of any of paragraphs 1-13.
26) A composition comprising any of the progenitor cell or a population of progenitor cell of paragraphs 15-17, or the isolated genetic engineered human cell or a population of genetic engineered human cells of paragraphs 18-20.
27) The composition of any of the preceding paragraphs, further comprising a pharmaceutically acceptable carrier.
28) The composition of any of the preceding paragraphs for use in an ex vivo method of producing a progenitor cell or a population of progenitor cells wherein the cells or the differentiated progeny therefrom have an altered genetic sequence, have corrected a IVS1-110G>A or IVS2-654C>T mutation, and/or have at least one genetic modification in the β-Globin gene.
29) The composition of any of the preceding paragraphs for use in an ex vivo method of producing an isolated genetic engineered human cell or a population of progenitor cells having an altered genetic sequence, having a corrected a IVS1-110G>A or IVS2-654C>T mutation, and/or having at least one genetic modification in the β-Globin gene.
30) A method for correcting an isolated progenitor cell or a population of isolated progenitor cells having a IVS1-110G>A or IVS2-654C>T mutation in the β-Globin gene, the method comprising contacting an isolated progenitor cell with an effective amount of any of the ribonucleoprotein (RNP) complexes of paragraphs 1-13, or the composition of paragraph 25, whereby the contacted cells or the differentiated progeny cells therefrom have corrected the IVS1-110G>A or IVS2-654C>T mutation in the β-Globin gene.
31) The method of any of the preceding paragraphs, wherein the isolated progenitor cell is a hematopoietic progenitor cell or a hematopoietic stem cell.
32) The method of any of the preceding paragraphs, wherein the hematopoietic progenitor is a cell of the erythroid lineage.
33) The method of any of the preceding paragraphs, wherein the isolated progenitor cell is an induced pluripotent stem cell.
34) The method of any of the preceding paragraphs, wherein the isolated progenitor cell is contacted ex vivo or in vitro.
35) A population of genetically edited progenitor cells produced by methods of any of paragraphs 30-34.
36) The population of any of the preceding paragraphs, wherein the genetically edited human cells are isolated.
37) A composition comprising isolated genetically edited human cells of paragraphs 35 and 36.
38) The composition of any of the preceding paragraphs, further comprising a pharmaceutically acceptable carrier.
39) A method of treating a disease associated with IVS1-110G>A or IVS2-654C>T mutation in the β-Globin gene, the method comprising, administering to a subject in need thereof any of the RNP complexes of any of paragraphs 1-13, any of the compositions of any of paragraphs 25-27 or 37-38, or the population of genetically edited progenitor cells of paragraphs 35-36.
40) The method of any of the preceding paragraphs, wherein the disease is thalassemia or β-thalassemia.
41) A RNP complex comprising a DNA-targeting endonuclease Cas9 protein and a guide RNA comprising the sequence of SEQ ID NO: 1 that targets and hybridizes to a target sequence on a DNA molecule.
42) A RNP complex comprising a DNA-targeting endonuclease Cas12a protein and a guide RNA comprising the sequence of SEQ ID NO: 3 that targets and hybridizes to a target sequence on a DNA molecule.
43) The RNP complex of any of the preceding paragraphs, wherein targeting and hybridizing corrects a IVS1-110G>A or mutation is present in the β-Globin gene
44) The RNP complex of any of the preceding paragraphs, wherein targeting and hybridizing corrects a IVS2-654C>T mutation is present in the β-Globin gene.

### EXAMPLES

### Example 1

### Introduction

Therapeutic genome editing is a promising treatment modality for inherited blood disorders in which genetic modification of autologous hematopoietic stem cells (HSCs) would result in durable correction of the hematopoietic system'. Gene editing is a byproduct of endogenous DNA damage repair pathways, such as homologous recombination (HR), nonhomologous end joining (NHEJ) and microhomology mediated end joining (MMEJ), acting on double strand breaks (DSBs) produced by programmable nucleases'. HR enables the precise templated repair of mutations. However the required co-delivery of an exogenous donor template, competing non-templated mutagenic repair and cell-cycle dependent activity are challenges to achieving therapeutic HR in quiescent HSCs³⁻⁵. NHEJ-based genetic disruption is a highly efficient and simple approach suitable when elimination of a functional sequence element will achieve a desired therapeutic outcome. Recently we have shown that the erythroid enhancer of BCL11A represents a therapeutic target for efficient genetic disruption by Cas9 in human HSCs with subsequent derepression of fetal hemoglobin (HbF) level [Wu et al.].

The β-thalassemias are a genetically heterogeneous set of conditions in which various mutations at HBB result in partial (β⁺) or complete (β⁰) loss of β-globin expression⁶. Several of the most common mutant alleles disrupt HBB splicing through the creation of aberrant splice sites. For example IVS1-110G>A (HBB:c.93-21G>A, rs35004220) is one of the most common mutations throughout the Mediterranean and Middle East and the most prevalent mutation in Cyprus⁷. This mutation generates a de novo splice acceptor site in HBB intron-1 that leads to an aberrant mRNA that includes 19 nt prior to the start of exon 2 resulting in a premature stop codon⁸. IVS2-654C>T (HBB:c.316-197C>T, rs34451549) is among the most frequent β-thalassemia mutations in East Asia⁹. This mutation creates a de novo splice donor site in HBB intron-2, resulting in an aberrant β-globin mRNA containing an additional 73 nt exon that produces a premature stop codon^{10,11}.

### Methods

### Protein Purification

Protein purification for 3×NLS-SpCas9 and LbCas12a-2×NLS used a common protocol. The generation and characterization of the 3×NLS-SpCas9 and LbCas12a-2×NLS constructs have been recently described (Wu et al. & Liu et al.). The pET21a plasmid backbone (Novagen) is used to drive the expression of each protein. The plasmid expressing 3×NLS-SpCas9 (or LbCas12a-2×NLS) was transformed into E. coli Rosetta (DE3) pLysS cells (EMD Millipore) for protein production. Cells were grown at 37° C. to an OD600 of ˜0.2, then shifted to 18° C. and induced at an OD600 of ˜0.4 for 16 hours with IPTG (1 mM final concentration). Following induction, cells were pelleted by centrifugation and then resuspended with Nickel-NTA buffer (20 mM TRIS+1 M NaCl+20 mM imidazole+1 mM TCEP, pH 7.5) supplemented with HALT Protease Inhibitor Cocktail, EDTA-Free (100×) [ThermoFisher] and lysed with M-110s Microfluidizer (Microfluidics) following the manufacturer's instructions. The protein was purified from the cell lysate using Ni-NTA resin, washed with five volumes of Nickel-NTA buffer and then eluted with elution buffer (20 mM TRIS, 500 mM NaCl, 500 mM Imidazole, 10% glycerol, pH 7.5). The 3×NLS-SpCas9 (or LbCas12a protein) was dialyzed overnight at 4° C. in 20 mM HEPES, 500 mM NaCl, 1 mM EDTA, 10% glycerol, pH 7.5. Subsequently, the protein was step dialyzed from 500 mM NaCl to 200 mM NaCl (final dialysis buffer: 20 mM HEPES, 200 mM NaCl, 1 mM EDTA, 10% glycerol, pH 7.5). Next, the protein was purified by cation exchange chromatography (5 ml HiTrap-S column, Buffer A 20 mM HEPES pH 7.5+1 mM TCEP, Buffer B 20 mM HEPES pH 7.5+1 M NaCl+1 mM TCEP, flow rate 5 ml/min, column volume 5 ml) followed by size-exclusion chromatography (SEC) on Superdex-200 (16/60) column (Isocratic size-exclusion running buffer=20 mM HEPES pH 7.5, 150 mM NaCl, 1 mM TCEP for 3×NLS-SpCas9 [or 20 mM HEPES pH 7.5, 300 mM NaCl, 1 mM TCEP for LbCas12a-2×NLS]). The primary protein peak from the SEC was concentrated in an Ultra-15 Centrifugal Filters Ultracel −30K (Amicon) to a concentration around 100 μM, based on absorbance at 280 nm. The purified protein quality was assessed by SDS-PAGE/Coomassie staining to be >95% pure and protein concentration was quantified with Pierce™ BCA Protein Assay Kit (ThermoFisher Scientific).

### Synthesis of IVS1-110A and IVS2-654T Specific Guide RNAs

Synthetic sgRNA to target SpCas9 to the IVS1-110A mutation site and AAVS1 control site were synthesized by Synthego with end protection containing the following guide sequences: GGGUGGGAAAAUAGACUAAU (SEQ ID NO: 1) and CUCCCUCCCAGGAUCCUCUC (SEQ ID NO: 2). Synthetic LbCas12a crRNAs to rs34451549T/rs1609812T TS1 and AAVS1 control site were synthesized by Integrated DNA Technologies (IDT) with proprietary modifications to each end of the crRNA (AITR1 on 5′ end and AITR2 on 3′ end):

LbCas12a rs34451549T/rs1609812T crRNA sequence:
(SEQ ID NO: 3)
/AlTR1/rUrArArUrUrUrCrUrArCrUrArArGrUrGrUrArGrAr
UrUrArUrGrCrArGrArArArUrArUrUrGrCrUrArUrUrArCrC/
AlTR2/
LbCas12a AAVS1 crRNA sequence:
(SEQ ID NO: 4)
/AlTR1/rUrArArUrUrUrCrUrArCrUrArArGrUrGrUrArGrAr
UrUrCrUrGrUrCrCrCrCrUrCrCrArCrCrCrCrArCrArGrUrG/
AlTR2/

### CD34− HSPC Isolation, RNP Electroporation, and Culture

Healthy human CD34⁺ HSPCs from mobilized peripheral blood of deidentified healthy donors were obtained from Fred Hutchinson Cancer Research Center, Seattle, Wash. CD34⁺ HSPCs of β-thalassemia patients were isolated from non-mobilized peripheral blood following Boston Children's Hospital institutional review board approval and patient informed consent. CD34⁺ HSPCs were enriched using the Miltenyi CD34 Microbead kit (Miltenyi Biotec). CD34⁺ HSPCs were cultured with X-VIVO 15 (Lonza, 04-418Q) supplemented with 100 ng ml⁻¹ human SCF, 100 ng ml⁻¹ human thrombopoietin (TPO) and 100 ng ml⁻¹ recombinant human Flt3-ligand (Flt3-L). After 24 hours of culture, HSPCs were electroporated with SpCas9 RNP or LbCas12a RNP. Electroporation was performed using Lonza 4D Nucleofector (V4XP-3032 for 20 μl Nucleocuvette Strips) following the manufacturer's instructions. The RNP complex was prepared by mixing Cas9 (100 pmol) and sgRNA (300 pmol, OD based quantification) or LbCas12a (400 pmol) and crRNA (400 pmol, OD based quantification) and incubating for 15 min at room temperature immediately before electroporation. 50K HSPCs resuspended in 20 μl P3 solution were mixed with RNP and transferred to a cuvette for electroporation with program EO-100. The electroporated cells were resuspended with X-VIVO media with cytokines and changed into erythroid differentiation medium (EDM) 24 h later for in vitro differentiation. EDM consisted of IMDM supplemented with 330 μg/ml human holo-transferrin, 10 μg/ml recombinant human insulin, 2 IU/ml heparin, 5% human solvent detergent pooled plasma AB, 3 IU/ml erythropoietin, 1% L-glutamine, and 1% penicillin/streptomycin. During days 0-7 of erythroid culture, EDM was further supplemented with 10⁻⁶ M hydrocortisone (Sigma), 100 ng ml⁻¹ human SCF, and 5 ng ml⁻¹ human IL-3 (R&D) as EDM-1. During days 7-11 of culture, EDM was supplemented with 100 ng ml⁻¹ human SCF only as EDM-2. During days 11-18 of culture, EDM had no additional supplements as EDM-3. Globin gene expression, hemoglobin HPLC, enucleation percentage, and cell size were assessed on day 18 of erythroid culture. For clonal liquid culture, edited CD34⁺ HSPCs were sorted into 150 μl EDM-1 in 96-well round bottom plates (Nunc) at one cell per well using FACSAria II. The cells were changed into EDM-2 media 7 days later in 96-well flat bottom plates (Nunc). After additional 4 days of culture, cells were changed into 150 μl-500 μl EDM-3 at a concentration of 1M/ml for further differentiation. After additional 7 days of culture, 1/10 of the cells were harvested for genotyping analysis, 1/10 of cells were harvested for RNA isolation with RNeasy Micro Kit (74004, Qiagen), and the remaining cells were processed by Hemolysate reagent (5125, Helena Laboratories).

### Sequence Analysis

Indel frequencies were measured from cells cultured in EDM 5 days after electroporation. Briefly, genomic DNA was extracted using the Qiagen Blood and Tissue kit. The HBB locus was amplified with KOD Hot Start DNA Polymerase and corresponding primers (Supplementary Table 4) using the following cycling conditions: 95 degrees for 3 min; 35 cycles of 95 degrees for 20 s, 60 degrees for 10 s, and 70 degrees for 10 s; 70 degrees for 5 min. Resulting PCR products were subjected to Sanger or Illumina deep sequencing. For the IVS1-110 target site analysis, a nested PCR approach was used, with the first round of 10 cycles, and then 1:10 dilution used as template for 35-cycle second round PCR. The deep sequencing data was analyzed by CRISPResso2 software [Clement et al, Nature Biotechnology, in press]¹². We predicted nuclease cleavage positions to be between position 3 and 4 counting from the NGG PAM for SpCas9 and between positions 20 and 21 counting from the TTTV PAM for LbCas12a. After alignment, the guide and predicted cleavage site are identified, and the window is set around the cleavage site to determine whether the read has been modified from the reference sequence. We used a minimum alignment identity of 60% and window size of 2 bp around the cleavage site for SpCas9 and 8 bp around the cleavage site for LbCas12a to account for the known staggered cleavage of the latter nuclease. We manually removed HBD-aligning reads and collapsed read counts by mutations so that reads with the same mutations adjacent the cleavage sites but differences in non-adjacent regions were classified as the same allele. These latter differences are mainly due to sequencing errors or trimming artifacts and not genome editing.

Amplicon sequences were aligned to respective pathogenic (IVS1-110G>A or IVS2-654C>T) reference sequences in CRISPResso2 to generate nucleotide quilts and allele plots. To determine allele-specific editing efficiency, we aligned reads to both pathogenic (IVS1-110G>A or IVS2-654C>T) and non-pathogenic (IVS1-110G or IVS2-654C) reference sequences. Many edited reads aligned equivalently to both reference alleles, so we regarded all edited reads as a single category and calculated editing efficiency using the number of unedited reads for each reference sequence. To account for all edited reads, we split reads with ambiguous alignments in CRISPResso2, which allocates ambiguously aligned reads equally toward both reference sequences. The total read count was then corrected by subtracting double-counted ambiguous alignments from the pool of edited reads. The percent unedited non-pathogenic (IVS1-110G and IVS2-654C), unedited pathogenic (IVS1-110G>A and IVS2-654C>T), and edited reads for each treatment and donor replicate were calculated by dividing the respective read counts by the total read number and multiplying by 100. We then calculated the percent edited reads for each reference allele and treatment by subtracting the percent unedited targeted (IVS1-110A and IVS2-654T RNPs) amplicons from the percent unedited control (sgAAVS1) amplicons. The percent edited reads were then divided by the percent unedited control reads and multiplied by 100 to find the editing efficiency of the IVS1-110A and IVS2-654T RNPs for their respective target sequences.

### Gene Expression

RNA isolation with RNeasy columns (Qiagen, 74106), reverse transcription with iScript cDNA synthesis kit (Bio-Rad, 170-8890), RT-qPCR with iQ SYBR Green Supermix (Bio-Rad, 170-8880) was performed to determine globin gene expression. Primers listed in Supplementary Table 4.

### Hemoglobin HPLC

Hemolysates were prepared from erythroid cells after 18 days of differentiation using Hemolysate reagent (5125, Helena Laboratories) and analyzed with D-10 Hemoglobin Analyzer (Bio-Rad). Because the D-10 Hemoglobin Analyzer is not calibrated to measure HbA2/Lepore/HbE, we calculated hemoglobin percentages from areas under the curve (AUCs) measured from HPLC traces in ImageJ (version 2.0.0-rc-68/1.52i). If the HbA peak exceeded the HPLC trace boundaries (e.g. the sgWS1-110G>A RNP-edited sample from the β⁺β^{Lepore} donor), the HbA AUC was extrapolated by dividing the HbF AUC by the HbF percent calculated by D-10 Hemoglobin Analyzer and multiplying the difference by the HbA percent calculated by the D-10 Hemoglobin Analyzer. HbF, HbA, HbA2/Lepore/HbE percentages were then calculated from the summed AUCs of the three peaks.

### Flow Cytometry

For HSC immunophenotyping, CD34⁺ HSPCs were incubated with Pacific Blue anti-human CD34 Antibody (343512, Biolegend), PE/Cy5 anti-human CD38 (303508, Biolegend), APC anti-human CD90 (328114, Biolegend), APC-H7 Mouse Anti-Human CD45RA (560674, BD Bioscience) and Brilliant Violet 510 anti-human Lineage Cocktail (348807, Biolegend). Cell sorting was performed on a FACSAria II machine (BD Biosciences). For enucleation analysis, cells were stained with 2 μg ml⁻¹ of the cell-permeable DNA dye Hoechst 33342 (Life Technologies) for 10 min at 37° C. The Hoechst 33342 negative cells were further gated for cell size analysis with forward scatter area (FSC-A) parameter. Relative cell size was calculated as median FSC-A of test samples as compared to healthy donor cells.

### Analysis of Linkage Between IVS2-654C>T and rs1609812

Samples from either individuals or family members underwent β-globin gene nucleotide sequencing at the Hemoglobin Diagnostic Reference Laboratory, Boston Medical Center. Individuals and families with at least one member found to have IVS2-654C>T mutation were further examined for their genotype at rs1609812.

### Analysis of Splice Sites

More than 20,000 splice sites from the human genome¹³ were used to generate a TRANSFAC format matrix. Weblogo3.0 (e.g., available on the world wide web at http://weblogo.threeplusone.com/create.cgi) was used to build a probability sequence logo for consensus splice acceptor and consensus splice donor sequences.

### Results

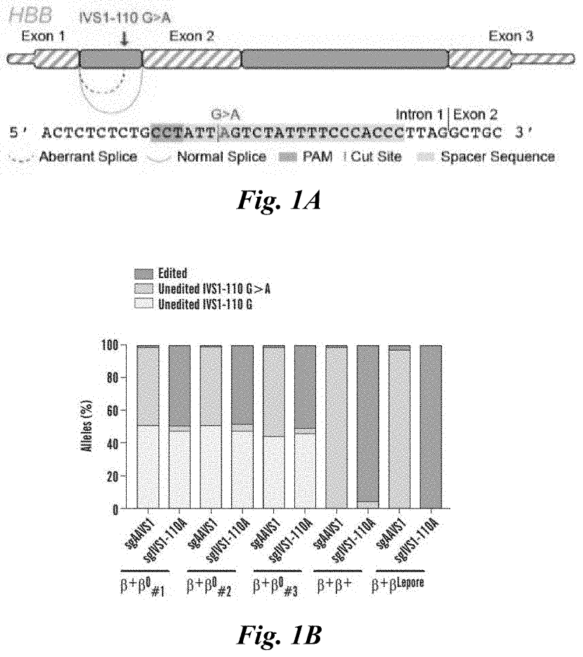

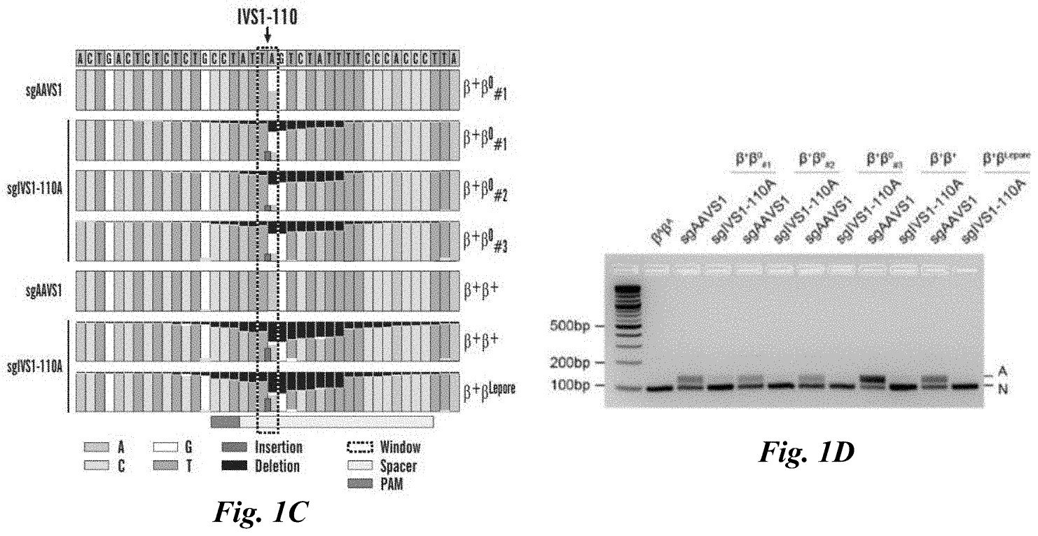

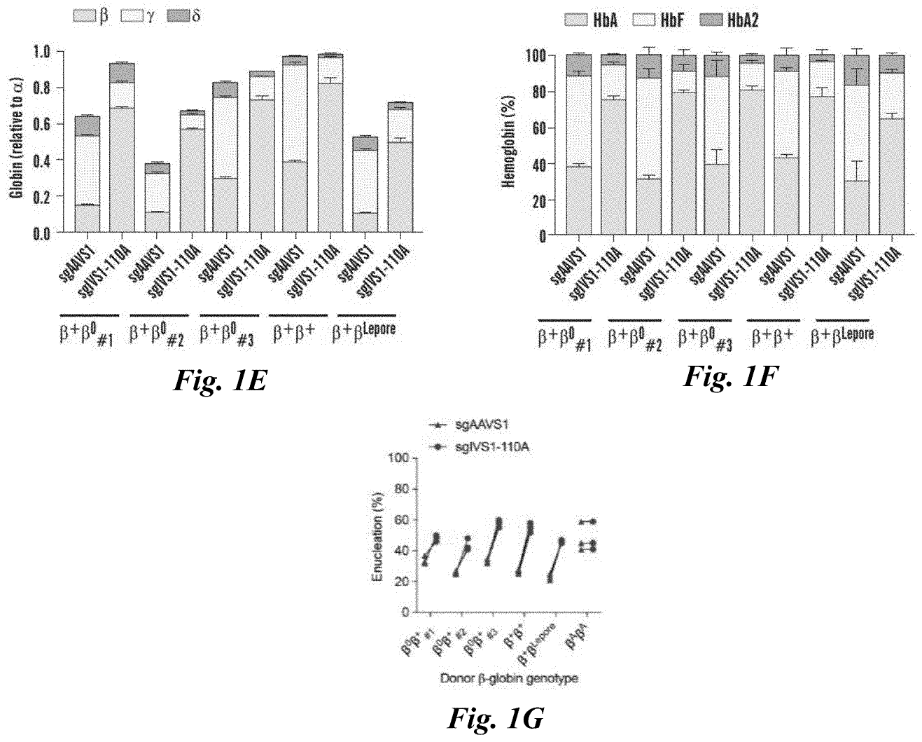

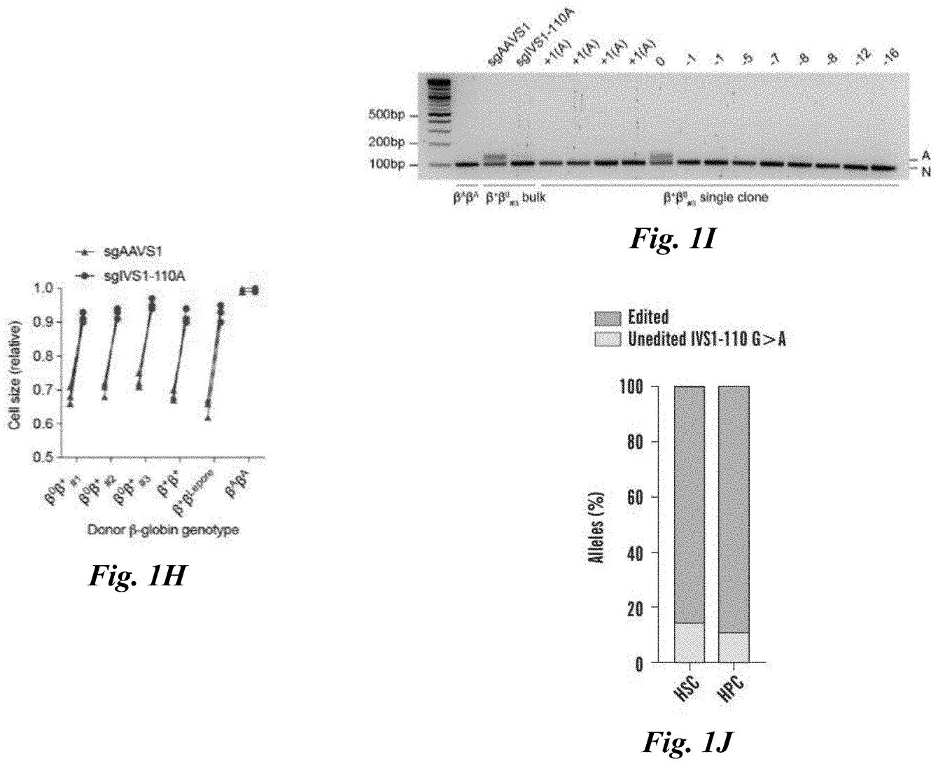

Recently we optimized conditions for high-efficiency S. pyogenes Cas9 (SpCas9) RNP editing of CD34+ HSPCs by electroporation [Wu et al.]. We hypothesized that RNP electroporation of patient CD34+ HSPCs could introduce high efficiency indels disrupting the aberrant splice sites, abrogating abnormal splicing and restoring β-globin expression. For the IVS1-110G>A target site, we identified a suitable SpCas9 NGG PAM that would direct DSB formation directly adjacent to the de novo splice acceptor site (FIG. 1A). We isolated non-mobilized peripheral blood CD34+ HSPCs from five transfusion-dependent β-thalassemia subjects carrying at least one HBB IVS1-110G>A allele. Three of these subjects were compound heterozygous for the IVS1-110G>A allele and a HBB null allele (β⁺β⁰_{#1}, β⁺β⁰_{#2}, β⁺β⁰_{#3}), one was homozygous for IVS1-110G>A (β⁺β⁺) and one was hemizygous for IVS1-110G>A with an HBB-HBD Lepore-Boston-Washington deletion of the other allele (β⁺β^{Lepore}) (Table 3). We electroporated CD34+ HSPCs from each of these donors with RNP composed of SpCas9-3×NLS protein and a chemically protected sgRNA complementary to the IVS1-110G>A mutant sequence (sgIVS1-110A). Then we subjected the electroporated cells to an 18-day 3-phase erythroid maturation protocol¹⁴. We found that SpCas9 mutagenesis was highly efficient, with mean 94.5% indel frequency at the IVS1-110G>A alleles within the treated population (FIG. 1B, 1C, 3A). The SpCas9 RNP preferentially targeted the IVS1-110G>A allele over the IVS1-110G allele due to a single base mismatch between the guide and target sequence. In the three evaluable compound heterozygous subjects (β⁺β⁰_{#1}, β⁺β⁰_{#2}, β⁺β⁰_{#3}), the IVS1-110G allele was inefficiently edited with mean 4.5% indel frequency despite nearly complete editing of IVS1-110G>A.

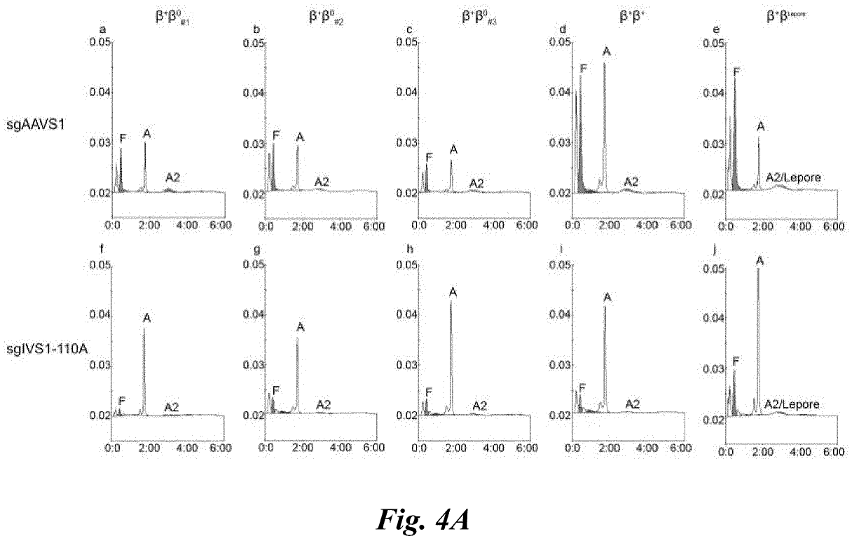

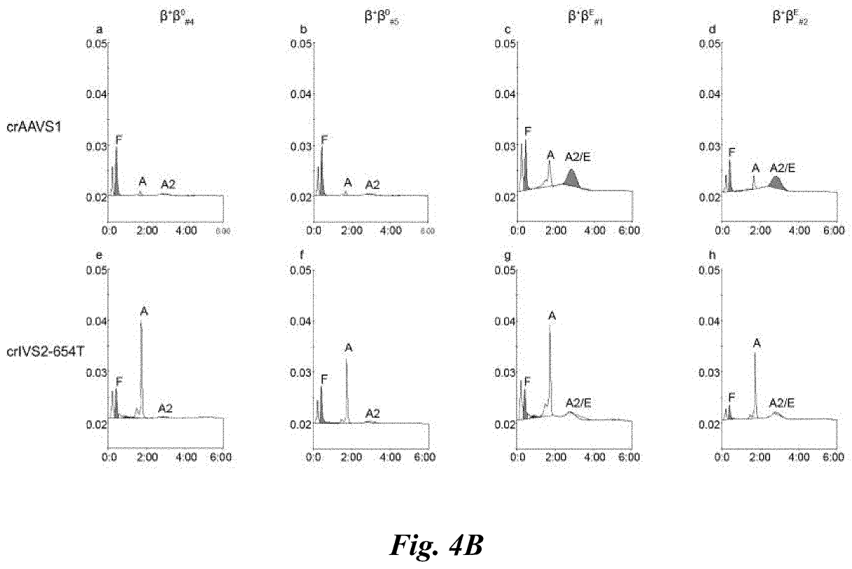

To test if genetic disruption of IVS1-110G>A within CD34+ HSPCs is sufficient to restore β-globin splicing and expression, we analyzed globin gene and hemoglobin protein expression following erythroid differentiation. We performed RT-PCR of β-globin mRNA, spanning the exon 1 to exon 2 junction, followed by gel electrophoresis (FIG. 1D). From a healthy donor sample, we observed a single band of the expected size (101 bp amplicon). However, for each of the five patients we found a lower mobility amplicon representing the expected aberrant splice product (118 bp amplicon). After SpCas9 sgIVS1-110A RNP editing, in each of the five patient donors, we observed a disappearance of the aberrant splice product and an increase in the intensity of the normal splice product. To quantify these changes we performed RT-qPCR with a β-globin primer pair specific to the normally spliced isoform. We observed that the expression of β-globin relative to β-globin increased from 20.8% in the sgAAVS1 controls compared to 66.2% in the sgIVS1-110A RNP edited samples (FIG. 1E). Hemoglobin quantification via HPLC showed a corresponding increase in the fraction of HbA from 36.4% to 75.6% after sgIVS1-110A RNP editing (FIGS. 1F, 4A and 4B). We hypothesized that restoration of globin chain balance would improve the quality of terminal erythroid maturation in vitro. We found for each of the five β-thalassemia patient donors that therapeutic editing restored the enucleation fraction and cell size to the normal range for differentiated erythroid cells, while the same editing had no effect on healthy donor differentiated erythroid cells (FIGS. 1G and 1H).

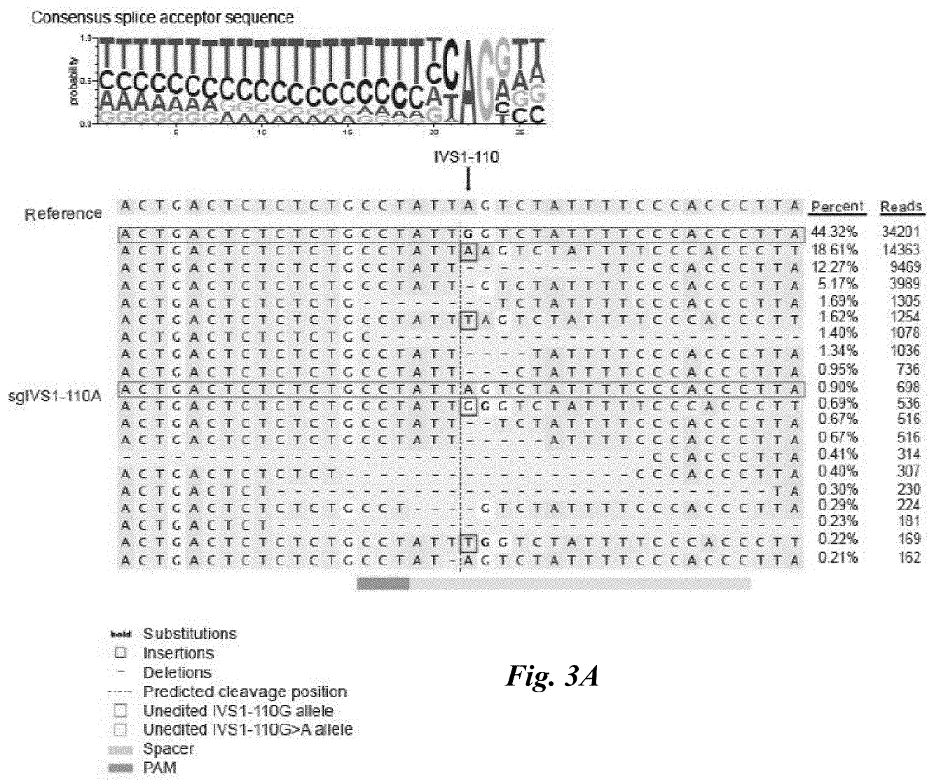

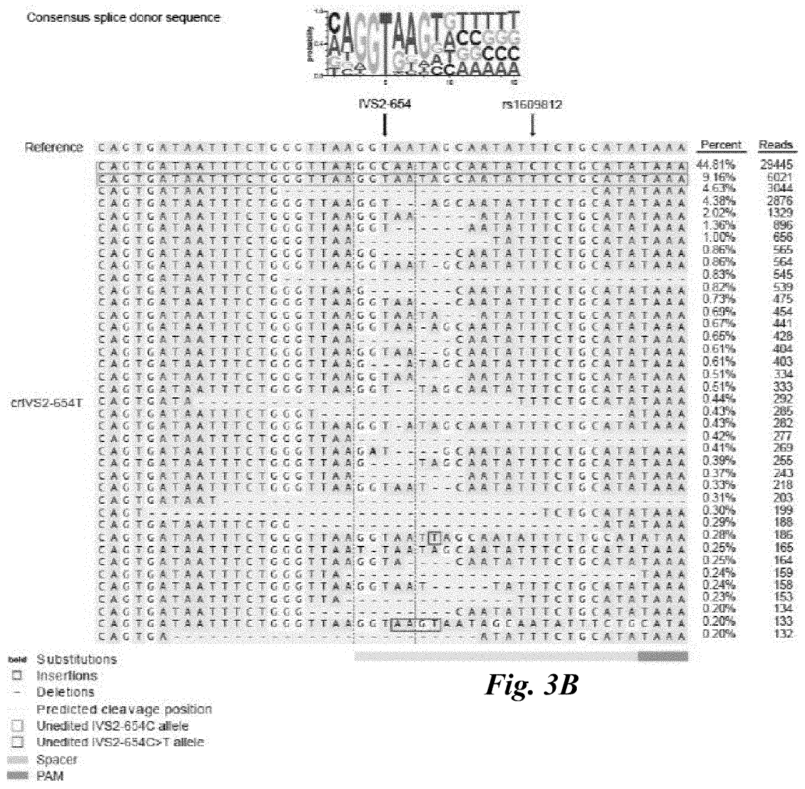

To correlate the genotype of individual edited alleles with β-globin expression, we sorted individual cells from donor β⁺β⁰_{#3} for clonal erythroid liquid culture following SpCas9 sgIVS1-110A RNP electroporation of CD34+ HPSCs. We performed paired Sanger genotyping and RT-PCR from 13 clones. In each clone we found that the WS1-110G allele was unedited. In one clone the IVS1-110G>A allele was unedited and the aberrant splicing product remained. In each of the other 12 clones a single indel was present in the IVS1-110G>A allele, ranging in length from a 1 bp insertion to a 16 bp deletion. In these twelve edited clones, the aberrant splice product was absent and only the normal splice product remained (FIG. 1I). These results demonstrate that even a +1(A) insertion adjacent to the IVS1-110G>A mutation was sufficient to restore normal β-globin splicing, consistent with nucleotide preference of the consensus splice acceptor site (FIG. 3A)¹⁵.

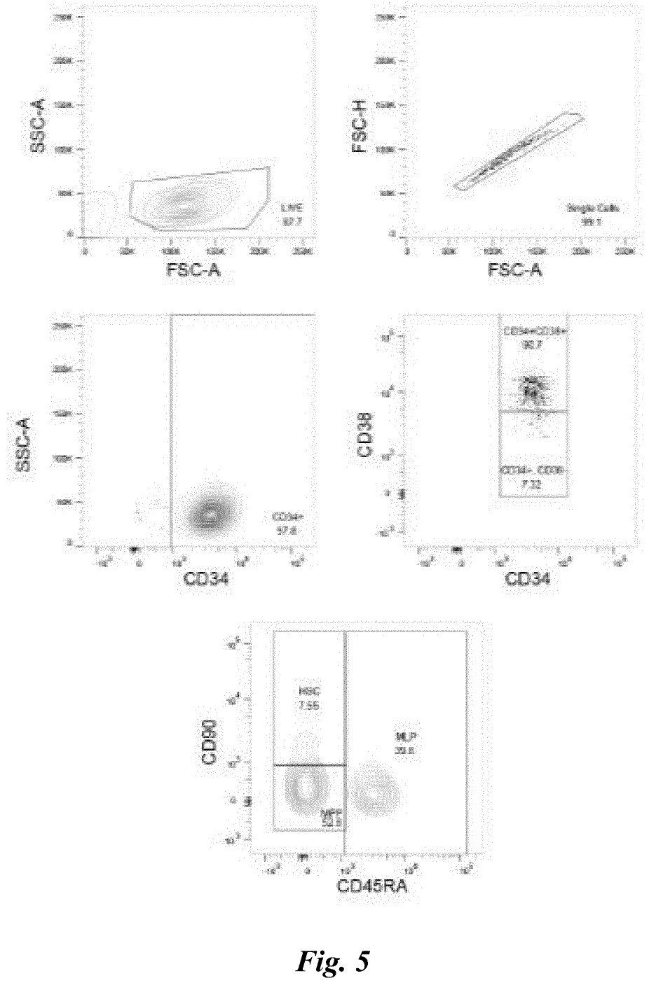

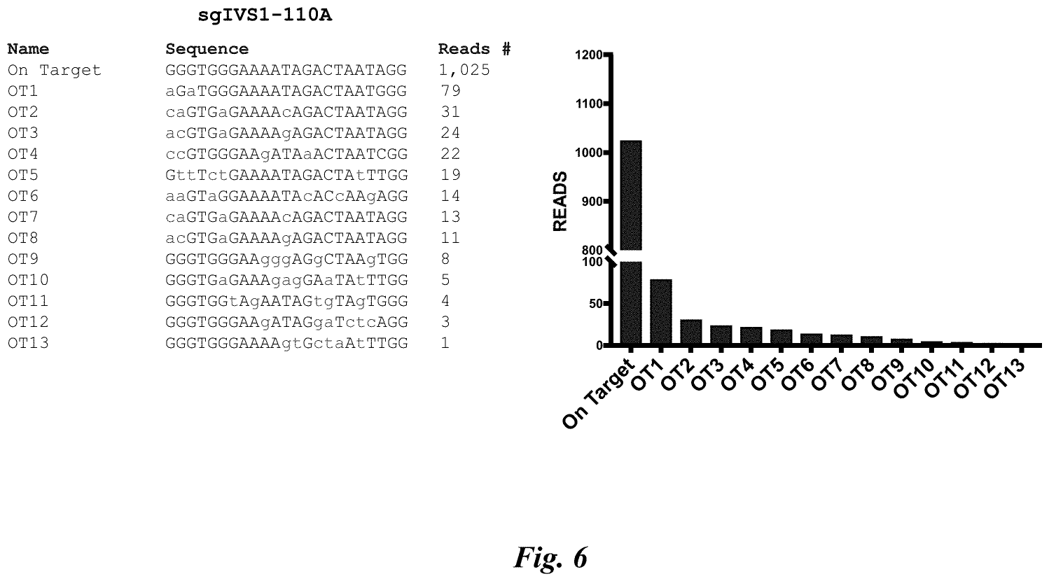

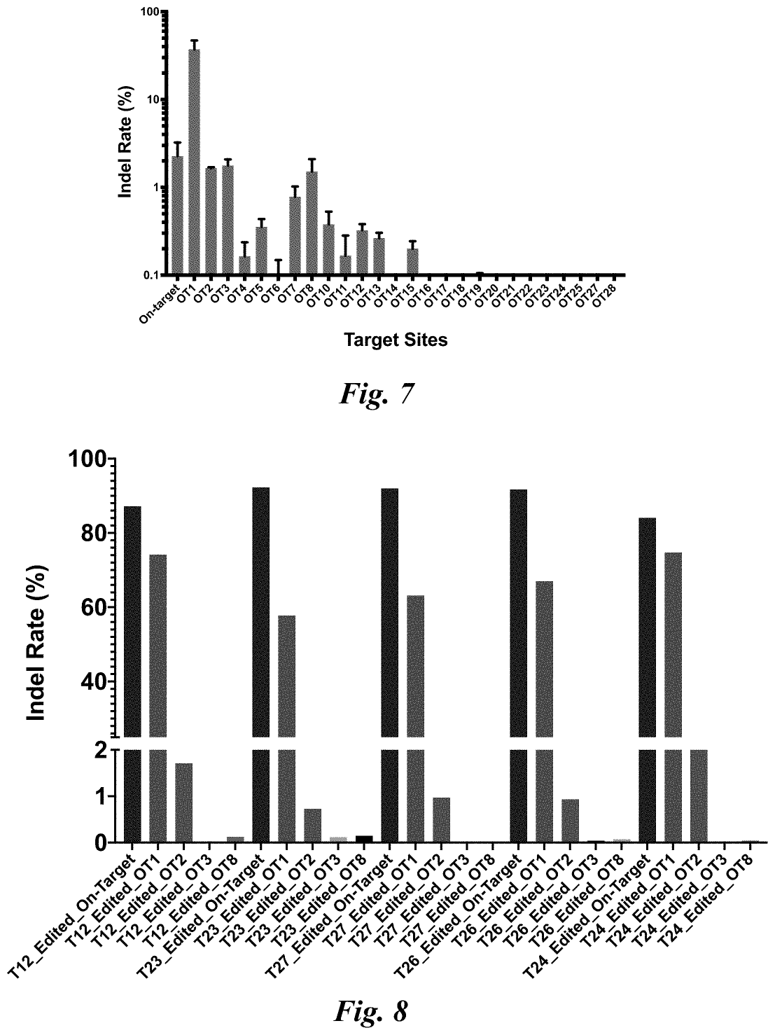

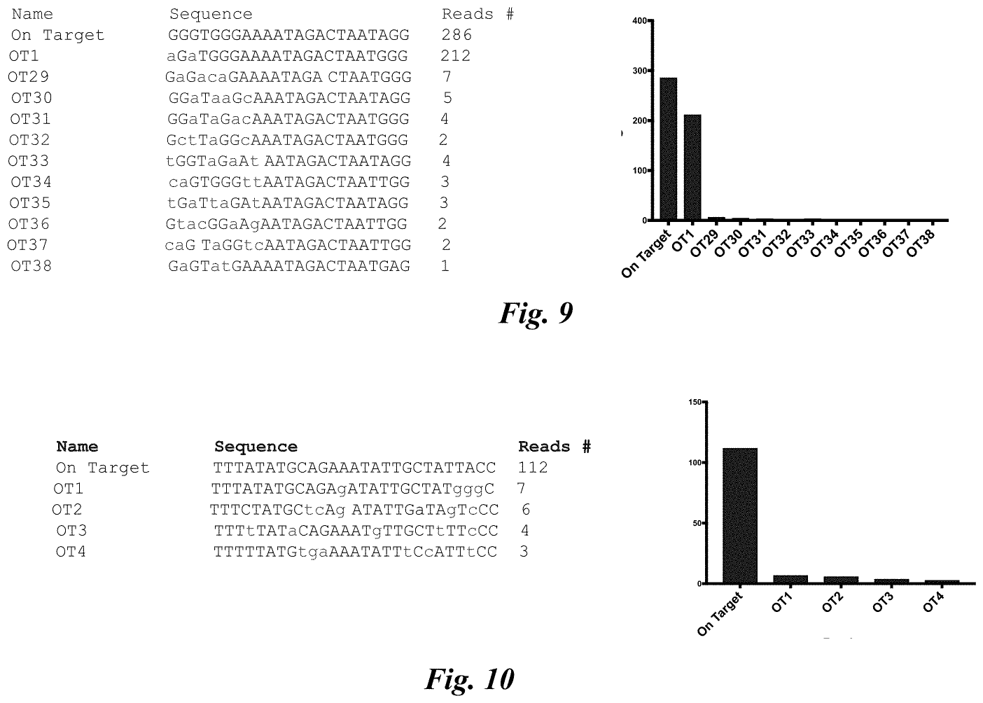

Since CD34+ HSPCs are a heterogeneous population of cells¹⁶, of which the majority are committed progenitors, we evaluated the editing in CD34+CD38+ hematopoietic progenitors (HPCs) as compared to an HSC enriched CD34+CD38−CD90+CD45RA-immunophenotype population (FIG. 5). We sorted the HSC and HPC populations 2 hours after SpCas9 RNP editing, which was performed 24 hours after CD34 HSPC isolation. We found that indel frequencies were similar in the HSC gated population (85.4%) as compared to the HPC gated population (88.9%) indicating that this strategy could efficiently generate therapeutic indels in HSCs.

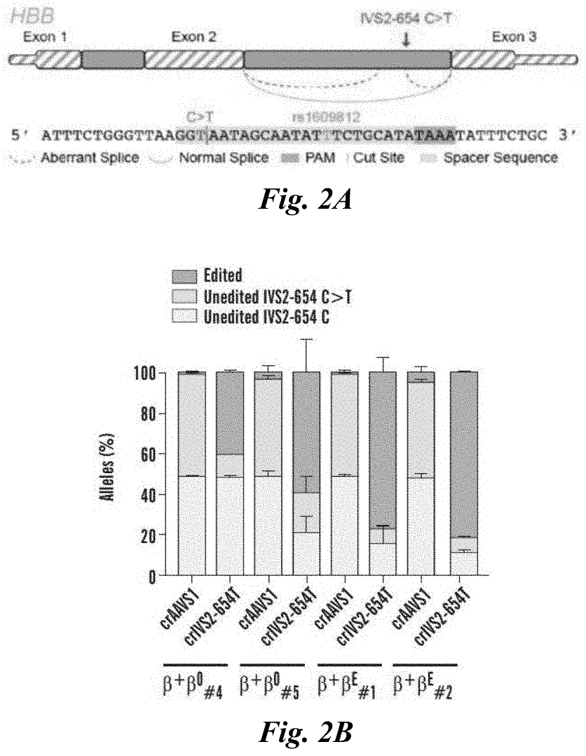

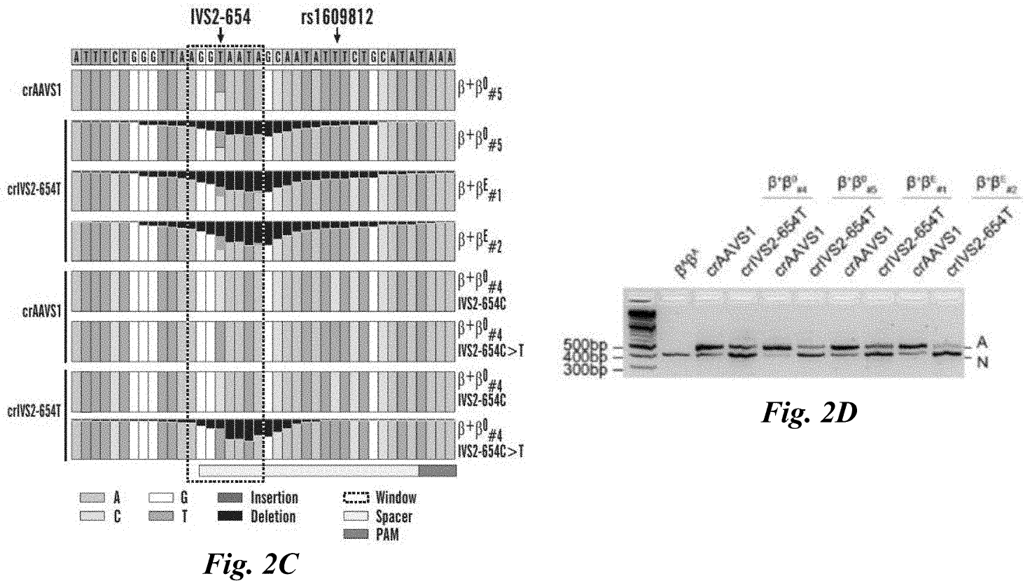

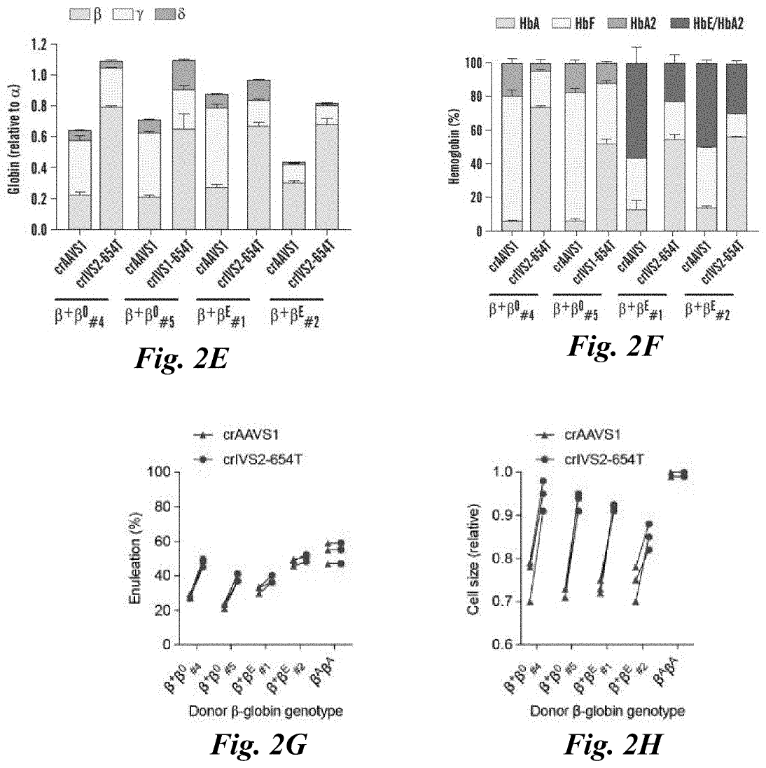

For IVS2-654C>T, there is no suitable NGG PAM neighboring the pathogenic mutation to target SpCas9 cleavage directly to the aberrant splice site. However, a TTTV PAM is appropriately positioned to target cleavage by L. bacterium ND2006 Cas12a/Cpf1 (LbCas12a) to the mutation (FIG. 2A). We identified four donors with transfusion-dependent β-thalassemia who carried the IVS2-654C>T mutation. Two of these subjects were compound heterozygous for IVS2-654C>T and a HBB null mutation (β⁺β⁰_{#4}, β⁺β⁰_{#5}) and two were compound heterozygous for the IVS2-654C>T mutation and a hemoglobin E mutation (β⁺β^{E}_{#1}, β⁺β^{E}_{#2}). One of these four subjects, β⁺β⁰_{#4}, was also heterozygous for the common SNP rs1609812 that overlaps the LbCas12a guide RNA sequence while the other three subjects were rs1609812-T/T homozygotes. Since Cas12a has been reported to be exquisitely specific with even a single mismatch to the guide sequence can prevent cleavage¹⁷, we determined the linkage of the IVS2-654C/T and rs1609812-C/T variants. We queried a set of 32 IVS2-654C>T alleles that had been ascertained through clinical sequencing for which linkage could be assigned. We found in each case IVS2-654T and rs1609812-T were found on the same haplotype, indicating perfect linkage disequilibrium between IVS2-654T and rs1609812-T (D′=1) (Table 4). Consistent with this analysis, deep sequencing confirmed that IVS2-654T was coinherited with rs1609812-T in the β⁺β⁰_{#4} donor.

We electroporated CD34+ HSPCs from each donor with LbCas12a RNP composed of LbCas12a protein and a crRNA complementary to the IVS2-654C>T mutant sequence (crIVS2-654T) and rs1609812-T. The cells were then subjected to the same erythroid differentiation protocol as described above. Editing by LbCas12a was efficient, with mean 77.0% indel frequency at the IVS2-654C>T alleles (FIGS. 2B, 2C, and 3B). The LbCas12a RNP was able to distinguish against alleles with IVS2-654C/rs1609812-C genotype (1.1% indels) but not against alleles with IVS2-654C/rs1609812-T genotype (67.6% indels). Each of the frequent indels at IVS2-654C>T were deletions overlapping the mutation that disrupt the aberrant splice donor site (FIG. 3B).

RT-PCR of β-globin, spanning the exon 2 to exon 3 junction, followed by gel electrophoresis, demonstrated the expression of normal and aberrant splice products in the differentiated erythroid cells from each affected donor (FIG. 2D). From a healthy donor sample, we observed only a single band of the expected size (395 bp amplicon). In the unedited patient samples we observed an additional band demonstrating the expected aberrant splice product (468 bp amplicon). After sgIVS2-654C>T RNP editing, in each of the four patient donors, we observed a reduction of the aberrant splice product and reciprocal increase of the normal splice product. We performed RT-qPCR with a β-globin primer pair specific to the normally spliced isoform to quantify the increase in properly spliced product. After editing we observed that the expression of β-globin relative to β-globin increased from 25.5% in crAAVS1-treated control to 70.1% in crIVS2-654T RNP edited samples (FIG. 2E). Hemoglobin quantification via HPLC showed an increase in the fraction of HbA from 9.9% to 59.1% after IVS2-654T editing (FIGS. 2F, 4A and 4B). For each of the four β-thalassemia patient donors, therapeutic editing of IVS2-654C>T by LbCas12a restored the enucleation fraction and cell size towards the normal range, while the same editing had no effect on healthy donor cells (FIGS. 2G and 2H).

### Discussion

In this study we demonstrate that CRISPR-Cas RNP electroporation of CD34+ HSPCs is an efficient strategy to disrupt aberrant splice site mutations. We demonstrate the application of this approach to yield phenotypic rescue of two common β-thalassemia mutations, IVS1-110G>A and IVS2-654C>T. These are among the most frequent mutations in specific populations affected by β-thalassemia, namely individuals of Mediterranean or East Asian ancestry, respectively. We find that all observed SpCas9-induced indels adjacent to the aberrant IVS1-110G>A splice acceptor site, including the frequent +1(A) insertion, restore normal splicing to β-globin. The overall efficiency of indels in CD34+ HSPCs plus the penetrance of splice site disruption indicate the robustness of this therapeutic editing strategy.

This is the first description to our knowledge of efficient RNP editing in CD34+ HSPCs with the Cas12a nuclease platform. It is possible that further optimization of the LbCas12a RNP could lead to even higher editing frequencies, analogous to the iterative improvements of SpCas9 RNP editing in CD34+ HSPCs reported over recent years⁵ [also Wu et al.]. Although the efficiency of mutagenesis by LbCas12a was modestly lower than SpCas9, the indels that were produced in HSPCs were almost exclusively deletions that span the mutation and aberrant splice site. This property of Cas12a proteins to produce slightly longer deletions and fewer insertions as compared to SpCas9 may make them especially useful for the targeted disruption of genomic elements'. Furthermore, we found that the IVS2-654C>T mutation was in perfect LD with the T allele at the common SNP rs1609812, indicating that a universal guide RNA design complementary to rs1609812-T can be used to target the IVS2-654C>T allele in the majority of affected individuals.

Alternative genetic therapies for the β-hemoglobin disorders have largely focused on globin gene addition, induction of HbF or repair of the HbS mutation¹⁹. A challenge to the development of gene repair approaches for the β-thalassemias has been the apparent need to develop individual repair strategies for each mutation in addition to intrinsic challenges of therapeutic HR^{20,21}. Here we propose that aberrant splice site disruption could be a simple and efficient strategy for β-thalassemia patients carrying at least one aberrant splice site mutation. Even monoallelic restoration of normal β-globin expression in a subset of HSCs could be sufficient to convert transfusion-dependent β-thalassemia to an asymptomatic hematologic condition²². We anticipate that this aberrant splice site disruption approach can be extended to additional mutations, disorders, and editing systems (Table 2).

### REFERENCES

1. Hoban, M. D. & Bauer, D. E. A genome editing primer for the hematologist. Blood 127, 2525-2535 (2016).
2. Chang, H. H. Y., Pannunzio, N. R., Adachi, N. & Lieber, M. R. Non-homologous DNA end joining and alternative pathways to double-strand break repair. Nat. Publ. Gr. 18, 495-506 (2017).
3. Mohrin, M. et al. Hematopoietic stem cell quiescence promotes error-prone DNA repair and mutagenesis. Cell Stem Cell 7, 174-185 (2010).
4. Genovese, P. et al. Targeted genome editing in human repopulating haematopoietic stem cells. Nature 510, 235-40 (2014).
5. Charlesworth, C. T. et al. Priming Human Repopulating Hematopoietic Stem and Progenitor Cells for Cas9/sgRNA Gene Targeting. Mol. Ther. Nucleic Acid 12, 89-104 (2018).
6. Origa, R. Beta-Thalassemia. GeneReviews 1-33 (2018).
7. Kountouris, P. et al. IthaGenes: An interactive database for haemoglobin variations and epidemiology. PLoS One 9, (2014).
8. Spritz, R. A. et al. Base substitution in an intervening sequence of a beta+-thalassemic human globin gene. Proc Natl Acad Sci USA 78, 2455-9 ST-Base substitution in an intervening s (1981).
9. Lau, Y.-L. et al. Prevalence and Genotypes of α- and β-Thalassemia Carriers in Hong Kong—Implications for Population Screening. N Engl. J. Med. 336, 1298-1301 (1997).
10. Cheng, T. C. et al. beta-Thalassemia in Chinese: use of in vivo RNA analysis and oligonucleotide hybridization in systematic characterization of molecular defects. Proc. Natl. Acad. Sci. U S. A. 81, 2821-5 (1984).
11. Takihara, Y. et al. One base substitution in IVS-2 causes a beta-plus thalassemia phenotype in a Chinese patient. Biochem. Biophys. Res. Commun. 121, 324-330 (1984).
12. Pinello, L. et al. CRISPResso: sequencing analysis toolbox for CRISPR-Cas9 genome editing. Nat. Biotechnol. 34, 695-697 (2016).
13. Ma, S. L. et al. Whole Exome Sequencing Reveals Novel PHEX Splice Site Mutations in Patients with Hypophosphatemic Rickets. 1-12 (2015). doi:10.1371/journal.pone.0130729
14. Giarratana, M. C. et al. Proof of principle for transfusion of in vitro-generated red blood cells. Blood 118, 5071-5079 (2011).
15. Yeo, G. & Burge, C. B. Maximum Entropy Modeling of Short Sequence Motifs with Applications to RNA Splicing Signals. J. Comput. Biol. 11, 377-394 (2004).
16. Notta, F. et al. Isolation of Single Human Hematopoietic Stem Cells Capable of Long-Term Multilineage Engraftment. Science (80-.). 333, 218-221 (2011).
17. Strohkendl, I., Saifuddin, F. A., Rybarski, J. R., Finkelstein, I. J. & Russell, R. Kinetic Basis for DNA Target Specificity of CRISPR-Cas12a. Mol. Cell 71, 816-824.e3 (2018).
18. Jinek, M. Cas9 versus Cas12a/Cpf1:Structure—function comparisons and implications for genome editing. 1-19 (2018). doi:10.1002/wrna.1481
19. Ferrari, G., Cavazzana, M. & Mavilio, F. Gene Therapy Approaches to Hemoglobinopathies. Hematol. Oncol. Clin. North Am. 31, 835-852 (2017).
20. Xu, P. et al. Both TALENs and CRISPR/Cas9 directly target the HBB IVS2-654 (C &gt; T) mutation in β-thalassemia-derived iPSCs. Sci. Rep. 5, 12065 (2015).
21. Antony, J. S. et al. Gene correction of HBB mutations in CD34 + hematopoietic stem cells using Cas9 mRNA and ssODN donors. 1, 1-7 (2018).
22. Andreani, M. et al. Quantitatively different red cell/nucleated cell chimerism in patients with long-term, persistent hematopoietic mixed chimerism after bone marrow transplantation for thalassemia major or sickle cell disease. Haematologica 96, 128-133 (2011).

TABLE 2
Aberrant splice site targeting in the thalassemias and other blood disorders

| | SEQ | | | | | |
|---|---|---|---|---|---|---|
| Spacer | ID NO | PAM | Nuclease | PAM_to_SNP | SNP_to_Cut | Strand |
| IVS1-110 (G>A), | | | | | | |
| HBB:c.93-21G>A | | | | | | |
| GGGTGGGAAAATAGACTAAT | 35 | AGG | Spycas9 | −4 | 0 | − |
| GGGAAAATAGACTAATAGGC | 36 | AGA | VQRSpyCas9_xCas9-3.7(NGA) | −8 | −4 | − |
| GAAAATAGACTAATAGGCAG | 37 | AGA | VQRSpyCas9_xCas9-3.7(NGA) | −10 | −6 | − |
| AAATAGACTAATAGGCAGAG | 38 | AGA | VQRSpyCas9_xCas9-3.7(NGA) | −12 | −8 | − |
| GGTGGGAAAATAGACTAATA | 39 | GGC | xCas9 3.7 (NGC) | −5 | −1 | − |
| ACTGACTCTCTCTGCCTATT | 40 | AGT | xCas9 3.7 (NGT) | 0 | −3 | + |
| GAAAATAGACTAATAGGCAGA | 41 | GAGAGT | SauCas9 | −11 | −7 | − |
| TGACTCTCTCTGCCTATTAGTCTA | 42 | TTTTCC | Nme2Cas9 | −6 | −2 | + |
| GACTCTCTCTGCCTATTAGTCTAT | 43 | TTTCCC | Nme2Cas9 | −7 | −3 | + |
| TCTCTCTGCCTATTAGTCTATTTT | 44 | CCCACC | Nme2Cas9 | −10 | −6 | + |
| CTCTCTGCCTATTAGTCTATTTTC | 45 | CCACCC | Nme2Cas9 | −11 | −7 | + |
| AGGCACTGACTCTCTCTGCCT | 46 | ATTAGT | KKHSauCas9 | 0 | −6 | + |
| AGCCTAAGGGTGGGAAAATAG | 47 | ACTAAT | KKHSauCas9 | 0 | −5 | − |
| IVS1-116 (T>G), | | | | | | |
| HBB:c.93-15T>G | | | | | | |
| GGGTGGGAAACTAGACCAAT | 48 | AGG | Spycas9 | −10 | −6 | − |
| CAGCCTAAGGGTGGGAAACT | 49 | AGA | VQRSpyCas9_xCas9-3.7(NGA) | −2 | 1 | − |
| GGTGGGAAACTAGACCAATA | 50 | GGC | xCas9 3.7 (NGC) | −11 | −7 | − |
| CTCTCTCTGCCTATTGGTCT | 51 | AGT | xCas9 3.7 (NGT) | 0 | −4 | + |
| TGACTCTCTCTGCCTATTGGTCTA | 52 | GTTTCC | Nme2Cas9 | 0 | −3 | + |
| GACTCTCTCTGCCTATTGGTCTAG | 53 | TTTCCC | Nme2Cas9 | −1 | 2 | + |
| TCTCTCTGCCTATTGGTCTAGTTT | 54 | CCCACC | Nme2Cas9 | −4 | 0 | + |
| CTCTCTGCCTATTGGTCTAGTTTC | 55 | CCACCC | Nme2Cas9 | −5 | −1 | + |
| ACCAGCAGCCTAAGGGTGGGAAAC | 56 | TAGACC | Nme2Cas9 | −1 | 2 | − |
| CTGACTCTCTCTGCCTATTGG | 57 | TCTAGT | KKHSauCas9 | 0 | −7 | + |
| AGCCTAAGGGTGGGAAACTAG | 58 | ACCAAT | KKHSauCas9 | −4 | 0 | − |
| IVS1-45 (G>C), | | | | | | |
| HBA1:c.95+45G>C | | | | | | |
| CACTGACTCTCTCTGCCTAT | 59 | AGG | Spycas9 | 0 | −3 | + |
| GGGTGGGAAAATAGACCTAT | 60 | AGG | Spycas9 | −3 | 0 | − |
| GGGAAAATAGACCTATAGGC | 61 | AGA | VQRSpyCas9_xCas9-3.7(NGA) | −7 | −3 | − |
| GAAAATAGACCTATAGGCAG | 62 | AGA | VQRSpyCas9_xCas9-3.7(NGA) | −9 | −5 | − |
| AAATAGACCTATAGGCAGAG | 63 | AGA | VQRSpyCas9_xCas9-3.7(NGA) | −11 | −7 | − |
| GGTGGGAAAATAGACCTATA | 64 | GGC | xCas9 3.7 (NGC) | −4 | 0 | − |
| ACTGACTCTCTCTGCCTATA | 65 | GGT | xCas9 3.7 (NGT) | −1 | 2 | + |
| GAAAATAGACCTATAGGCAGA | 66 | GAGAGT | SauCas9 | −10 | −6 | − |
| TGACTCTCTCTGCCTATAGGTCTA | 67 | TTTTCC | Nme2Cas9 | −7 | −3 | + |
| GACTCTCTCTGCCTATAGGTCTAT | 68 | TTTCCC | Nme2Cas9 | −8 | −4 | + |
| TCTCTCTGCCTATAGGTCTATTTT | 69 | CCCACC | Nme2Cas9 | −11 | −7 | + |
| CTCTCTGCCTATAGGTCTATTTTC | 70 | CCACCC | Nme2Cas9 | −12 | −8 | + |
| AGGCACTGACTCTCTCTGCCT | 71 | ATAGGT | KKHSauCas9 | 0 | −5 | + |
| IVS1-5 (G>A), | | | | | | |
| HBB:c.92+5G>A | | | | | | |
| CCCTGGGCAGGTTGTTATCA | 72 | AGG | Spycas9 | −6 | −2 | + |
| ACCTTGATAACAACCTGCCC | 73 | AGG | Spycas9 | −11 | −7 | − |
| CCTTGATAACAACCTGCCCA | 74 | GGG | Spycas9 | −12 | −8 | − |
| TTGTAACCTTGATAACAACC | 75 | TGC | xCas9 3.7 (NGC) | −6 | −2 | − |
| TGGTGAGGCCCTGGGCAGGT | 76 | TGT | xCas9 3.7 (NGT) | 0 | −5 | + |
| CCTGGGCAGGTTGTTATCAA | 77 | GGT | xCas9 3.7 (NGT) | −7 | −3 | + |
| AACCTGTCTTGTAACCTTGATAA | 78 | TTA | FnCpf1 | 23 | 0 | − |
| TAACCTTGATAACAACCTGCCCA | 79 | TTG | FnCpf1 | 12 | 7 | − |
| TAAACCTGTCTTGTAACCTTGATA | 80 | ACAACC | Nme2Cas9 | 0 | −3 | − |
| CCTGTCTTGTAACCTTGATAACAA | 81 | CCTGCC | Nme2Cas9 | −4 | 0 | − |
| CTGTCTTGTAACCTTGATAACAAC | 82 | CTGCCC | Nme2Cas9 | −5 | −1 | − |
| TGTAACCTTGATAACAACCTGCCC | 83 | AGGGCC | Nme2Cas9 | −11 | −7 | − |
| AGGCCCTGGGCAGGTTGTTAT | 84 | CAAGGT | KKHSauCas9 | −4 | 0 | + |
| IVS1-5 (G>C), | | | | | | |
| HBB:c.92+5G>C | | | | | | |
| CCCTGGGCAGGTTGCTATCA | 85 | AGG | Spycas9 | −6 | −2 | + |
| ACCTTGATAGCAACCTGCCC | 86 | AGG | Spycas9 | −11 | −7 | − |
| CCTTGATAGCAACCTGCCCA | 87 | GGG | Spycas9 | −12 | −8 | − |
| TGGTGAGGCCCTGGGCAGGT | 88 | TGC | xCas9 3.7 (NGC) | 0 | −5 | + |
| ACCTGTCTTGTAACCTTGAT | 89 | AGC | xCas9 3.7 (NGC) | 0 | −4 | − |
| TTGTAACCTTGATAGCAACC | 90 | TGC | xCas9 3.7 (NGC) | −6 | −2 | − |
| CCTGGGCAGGTTGCTATCAA | 91 | GGT | xCas9 3.7 (NGT) | −7 | −3 | + |
| AACCTGTCTTGTAACCTTGATAG | 92 | TTA | FnCpf1 | 23 | 0 | − |
| TAACCTTGATAGCAACCTGCCCA | 93 | TTG | FnCpf1 | 12 | 7 | − |
| TAAACCTGTCTTGTAACCTTGATA | 94 | GCAACC | Nme2Cas9 | 0 | −3 | − |
| CCTGTCTTGTAACCTTGATAGCAA | 95 | CCTGCC | Nme2Cas9 | −4 | 0 | − |
| CTGTCTTGTAACCTTGATAGCAAC | 96 | CTGCCC | Nme2Cas9 | −5 | −1 | − |
| TGTAACCTTGATAGCAACCTGCCC | 97 | AGGGCC | Nme2Cas9 | −11 | −7 | − |
| AGGCCCTGGGCAGGTTGCTAT | 98 | CAAGGT | KKHSauCas9 | −4 | 0 | + |
| IVS1-5 (G>T), | | | | | | |
| HBB:c.92+5G>T | | | | | | |
| CCCTGGGCAGGTTGTTATCA | 99 | AGG | Spycas9 | −6 | −2 | + |
| ACCTTGATAACAACCTGCCC | 100 | AGG | Spycas9 | −11 | −7 | − |
| CCTTGATAACAACCTGCCCA | 101 | GGG | Spycas9 | −12 | −8 | − |
| TTGTAACCTTGATAACAACC | 102 | TGC | xCas9 3.7 (NGC) | −6 | −2 | − |
| TGGTGAGGCCCTGGGCAGGT | 103 | TGT | xCas9 3.7 (NGT) | 0 | −5 | + |
| CCTGGGCAGGTTGTTATCAA | 104 | GGT | xCas9 3.7 (NGT) | −7 | −3 | + |
| AACCTGTCTTGTAACCTTGATAA | 105 | TTA | FnCpf1 | 23 | 0 | − |
| TAACCTTGATAACAACCTGCCCA | 106 | TTG | FnCpf1 | 12 | 7 | − |
| TAAACCTGTCTTGTAACCTTGATA | 107 | ACAACC | Nme2Cas9 | 0 | −3 | − |
| CCTGTCTTGTAACCTTGATAACAA | 108 | CCTGCC | Nme2Cas9 | −4 | 0 | − |
| CTGTCTTGTAACCTTGATAACAAC | 109 | CTGCCC | Nme2Cas9 | −5 | −1 | − |
| TGTAACCTTGATAACAACCTGCCC | 110 | AGGGCC | Nme2Cas9 | −11 | −7 | − |
| AGGCCCTGGGCAGGTTGTTAT | 111 | CAAGGT | KKHSauCas9 | −4 | 0 | + |
| IVS1-5 (G>A), | | | | | | |
| HBA1:c.95+5G>A | | | | | | |
| CCCTGGGCAGGTTGTTATCA | 112 | AGG | Spycas9 | −6 | −2 | + |
| ACCTTGATAACAACCTGCCC | 113 | AGG | Spycas9 | −11 | −7 | − |
| CCTTGATAACAACCTGCCCA | 114 | GGG | Spycas9 | −12 | −8 | − |
| TTGTAACCTTGATAACAACC | 115 | TGC | xCas9 3.7 (NGC) | −6 | −2 | − |
| TGGTGAGGCCCTGGGCAGGT | 116 | TGT | xCas9 3.7 (NGT) | 0 | −5 | + |
| CCTGGGCAGGTTGTTATCAA | 117 | GGT | xCas9 3.7 (NGT) | −7 | −3 | + |
| AACCTGTCTTGTAACCTTGATAA | 118 | TTA | FnCpf1 | 23 | 0 | − |
| TAACCTTGATAACAACCTGCCCA | 119 | TTG | FnCpf1 | 12 | 7 | − |
| TAAACCTGTCTTGTAACCTTGATA | 120 | ACAACC | Nme2Cas9 | 0 | −3 | − |
| CCTGTCTTGTAACCTTGATAACAA | 121 | CCTGCC | Nme2Cas9 | −4 | 0 | − |
| CTGTCTTGTAACCTTGATAACAAC | 122 | CTGCCC | Nme2Cas9 | −5 | −1 | − |
| TGTAACCTTGATAACAACCTGCCC | 123 | AGGGCC | Nme2Cas9 | −11 | −7 | − |
| AGGCCCTGGGCAGGTTGTTAT | 124 | CAAGGT | KKHSauCas9 | −4 | 0 | + |
| IVS1-5 (G>A), | | | | | | |
| HBA2:c.95+5G>A | | | | | | |
| GTGCGGAGGCCCTGGAGAGG | 125 | TGG | Spycas9 | 0 | −5 | + |
| TGCGGAGGCCCTGGAGAGGT | 126 | GGG | Spycas9 | 0 | −4 | + |
| GCGGAGGCCCTGGAGAGGTG | 127 | GGG | Spycas9 | 0 | −3 | + |
| GGAGGGAGCCCCACCTCTCC | 128 | AGG | Spycas9 | −10 | −6 | − |
| GAGGGAGCCCCACCTCTCCA | 129 | GGG | Spycas9 | −11 | −7 | − |
| CGGAGGCCCTGGAGAGGTGG | 130 | GGC | xCas9 3.7 (NGC) | −1 | 2 | + |
| AGGGAGCCCCACCTCTCCAG | 131 | GGC | xCas9 3.7 (NGC) | −12 | −8 | − |
| GTGCGGAGGCCCTGGAGAGG | 132 | TGGGG | St3Cas9 | 0 | −5 | + |
| GTGCGGAGGCCCTGGAGAGGTGG | 133 | TATG | Cpf1RVR | 23 | 0 | + |
| GGTGCGGAGGCCCTGGAGAGGTGG | 134 | GGCTCC | Nme2Cas9 | −1 | 2 | + |
| GTGCGGAGGCCCTGGAGAGGTGGG | 135 | GCTCCC | Nme2Cas9 | −2 | 1 | + |
| CGGAGGCCCTGGAGAGGTGGGGCT | 136 | CCCTCC | Nme2Cas9 | −5 | −1 | + |
| GGAGGCCCTGGAGAGGTGGGGCTC | 137 | CCTCCC | Nme2Cas9 | −6 | −2 | + |
| GAGGCCCTGGAGAGGTGGGGCTCC | 138 | CTCCCC | Nme2Cas9 | −7 | −3 | + |
| GAGCCCGGGTCGGAGCAGGGGAGG | 139 | GAGCCC | Nme2Cas9 | 0 | −8 | − |
| AGCCCGGGTCGGAGCAGGGGAGGG | 140 | AGCCCC | Nme2Cas9 | 0 | −7 | − |
| CCGGGTCGGAGCAGGGGAGGGAGC | 141 | CCCACC | Nme2Cas9 | 0 | −4 | − |
| TCGGAGCAGGGGAGGGAGCCCCAC | 142 | CTCTCC | Nme2Cas9 | −4 | 0 | − |
| CAGGGGAGGGAGCCCCACCTCTCC | 143 | AGGGCC | Nme2Cas9 | −10 | −6 | − |
| IVS1-55 (G>A), | | | | | | |
| HBA2:c.95+55G>A | | | | | | |
| GGACGGTTGAGGGTGGTCTG | 144 | TGG | Spycas9 | −4 | 0 | − |
| GACGGTTGAGGGTGGTCTGT | 145 | GGG | Spycas9 | −5 | −1 | − |
| TTGAGGGTGGTCTGTGGGTC | 146 | CGG | Spycas9 | −10 | −6 | − |
| TGAGGGTGGTCTGTGGGTCC | 147 | GGG | Spycas9 | −11 | −7 | − |
| GAGGGTGGTCTGTGGGTCCG | 148 | GGCG | VRER SpyCas9 | −12 | −8 | − |
| CCTCGCCCGCCCGGACCCAC | 149 | AGA | VQRSpyCas9_xCas9-3.7(NGA) | 0 | −5 | + |
| GAGGGTGGTCTGTGGGTCCG | 150 | GGC | xCas9 3.7 (NGC) | −12 | −8 | − |
| ACCCACAGACCACCCTCAAC | 151 | CGT | xCas9 3.7 (NGT) | −12 | −8 | + |
| GGGCCAGGACGGTTGAGGGT | 152 | GGT | xCas9 3.7 (NGT) | 0 | −5 | − |
| CAGGACGGTTGAGGGTGGTC | 153 | TGT | xCas9 3.7 (NGT) | −2 | 1 | − |
| ACGGTTGAGGGTGGTCTGTG | 154 | GGT | xCas9 3.7 (NGT) | −6 | −2 | − |
| CAGGACGGTTGAGGGTGGTCT | 155 | GTGGGT | SauCas9 | −3 | 0 | − |
| TGAGGGTGGTCTGTGGGTCC | 156 | GGGCG | St3Cas9 | −11 | −7 | − |
| GGGCCAGGACGGTTGAGGGTGGT | 157 | TCCG | Cpf1 RR | 23 | 0 | − |
| GGGCTCCTCGCCCGCCCGGACCCA | 158 | CAGACC | Nme2Cas9 | 0 | −6 | + |
| CTCCTCGCCCGCCCGGACCCACAG | 159 | ACCACC | Nme2Cas9 | 0 | −3 | + |
| TCCTCGCCCGCCCGGACCCACAGA | 160 | CCACCC | Nme2Cas9 | −1 | 2 | + |
| CCCGCCCGGACCCACAGACCACCC | 161 | TCAACC | Nme2Cas9 | −7 | −3 | + |
| CCCGGACCCACAGACCACCCTCAA | 162 | CCGTCC | Nme2Cas9 | −11 | −7 | + |
| CCAGGACGGTTGAGGGTGGTCTGT | 163 | GGGTCC | Nme2Cas9 | −5 | −1 | − |
| TCCGGGGCCAGGACGGTTGAG | 164 | GGTGGT | KKHSauCas9 | 0 | −8 | − |
| IVS1-6 (T>C), | | | | | | |
| HBB:c.92+6T>C | | | | | | |
| GGACGGTTGAGGGTGGTCTG | 165 | TGG | Spycas9 | −4 | 0 | − |
| GACGGTTGAGGGTGGTCTGT | 166 | GGG | Spycas9 | −5 | −1 | − |
| TTGAGGGTGGTCTGTGGGTC | 167 | CGG | Spycas9 | −10 | −6 | − |
| TGAGGGTGGTCTGTGGGTCC | 168 | GGG | Spycas9 | −11 | −7 | − |
| GAGGGTGGTCTGTGGGTCCG | 169 | GGCG | VRER SpyCas9 | −12 | −8 | − |
| CCTCGCCCGCCCGGACCCAC | 170 | AGA | VQRSpyCas9_xCas9-3.7(NGA) | 0 | −5 | + |
| GAGGGTGGTCTGTGGGTCCG | 171 | GGC | xCas9 3.7 (NGC) | −12 | −8 | − |
| ACCCACAGACCACCCTCAAC | 172 | CGT | xCas9 3.7 (NGT) | −12 | −8 | + |
| GGGCCAGGACGGTTGAGGGT | 173 | GGT | xCas9 3.7 (NGT) | 0 | −5 | − |
| CAGGACGGTTGAGGGTGGTC | 174 | TGT | xCas9 3.7 (NGT) | −2 | 1 | − |
| ACGGTTGAGGGTGGTCTGTG | 175 | GGT | xCas9 3.7 (NGT) | −6 | −2 | − |
| CAGGACGGTTGAGGGTGGTCT | 176 | GTGGGT | SauCas9 | −3 | 0 | − |
| TGAGGGTGGTCTGTGGGTCC | 177 | GGGCG | St3Cas9 | −11 | −7 | − |
| GGGCCAGGACGGTTGAGGGTGGT | 178 | TCCG | Cpf1 RR | 23 | 0 | − |
| GGGCTCCTCGCCCGCCCGGACCCA | 179 | CAGACC | Nme2Cas9 | 0 | −6 | + |
| CTCCTCGCCCGCCCGGACCCACAG | 180 | ACCACC | Nme2Cas9 | 0 | −3 | + |
| TCCTCGCCCGCCCGGACCCACAGA | 181 | CCACCC | Nme2Cas9 | −1 | 2 | + |
| CCCGCCCGGACCCACAGACCACCC | 182 | TCAACC | Nme2Cas9 | −7 | −3 | + |
| CCCGGACCCACAGACCACCCTCAA | 183 | CCGTCC | Nme2Cas9 | −11 | −7 | + |
| CCAGGACGGTTGAGGGTGGTCTGT | 184 | GGGTCC | Nme2Cas9 | −5 | −1 | − |
| TCCGGGGCCAGGACGGTTGAG | 185 | GGTGGT | KKHSauCas9 | 0 | −8 | − |
| IVS1-7 (A>G), | | | | | | |
| HBB:c.92+7A>G | | | | | | |
| GGACGGTTGAGGGTGGTCTG | 186 | TGG | Spycas9 | −4 | 0 | − |
| GACGGTTGAGGGTGGTCTGT | 187 | GGG | Spycas9 | −5 | −1 | − |
| TTGAGGGTGGTCTGTGGGTC | 188 | CGG | Spycas9 | −10 | −6 | − |
| TGAGGGTGGTCTGTGGGTCC | 189 | GGG | Spycas9 | −11 | −7 | − |
| GAGGGTGGTCTGTGGGTCCG | 190 | GGCG | VRER SpyCas9 | −12 | −8 | − |
| CCTCGCCCGCCCGGACCCAC | 191 | AGA | VQRSpyCas9_xCas9-3.7(NGA) | 0 | −5 | + |
| GAGGGTGGTCTGTGGGTCCG | 192 | GGC | xCas9 3.7 (NGC) | −12 | −8 | − |
| ACCCACAGACCACCCTCAAC | 193 | CGT | xCas9 3.7 (NGT) | −12 | −8 | + |
| GGGCCAGGACGGTTGAGGGT | 194 | GGT | xCas9 3.7 (NGT) | 0 | −5 | − |
| CAGGACGGTTGAGGGTGGTC | 195 | TGT | xCas9 3.7 (NGT) | −2 | 1 | − |
| ACGGTTGAGGGTGGTCTGTG | 196 | GGT | xCas9 3.7 (NGT) | −6 | −2 | − |
| CAGGACGGTTGAGGGTGGTCT | 197 | GTGGGT | SauCas9 | −3 | 0 | − |
| TGAGGGTGGTCTGTGGGTCC | 198 | GGGCG | St3Cas9 | −11 | −7 | − |
| GGGCCAGGACGGTTGAGGGTGGT | 199 | TCCG | Cpfl RR | 23 | 0 | − |
| GGGCTCCTCGCCCGCCCGGACCCA | 200 | CAGACC | Nme2Cas9 | 0 | −6 | + |
| CTCCTCGCCCGCCCGGACCCACAG | 201 | ACCACC | Nme2Cas9 | 0 | −3 | + |
| TCCTCGCCCGCCCGGACCCACAGA | 202 | CCACCC | Nme2Cas9 | −1 | 2 | + |
| CCCGCCCGGACCCACAGACCACCC | 203 | TCAACC | Nme2Cas9 | −7 | −3 | + |
| CCCGGACCCACAGACCACCCTCAA | 204 | CCGTCC | Nme2Cas9 | −11 | −7 | + |
| CCAGGACGGTTGAGGGTGGTCTGT | 205 | GGGTCC | Nme2Cas9 | −5 | −1 | − |
| TCCGGGGCCAGGACGGTTGAG | 206 | GGTGGT | KKHSauCas9 | 0 | −8 | − |
| IVS1-7 (A>T), | | | | | | |
| HBB:c.92+7A>T | | | | | | |
| CCCTGGGCAGGTTGGTTTCA | 207 | AGG | Spycas9 | −4 | 0 | − |
| AGGTTGGTTTCAAGGTTACA | 208 | AGA | VQRSpyCas9_xCas9-3.7(NGA) | −12 | −8 | − |
| TTGTAACCTTGAAACCAACC | 209 | TGC | xCas9 3.7 (NGC) | −8 | −4 | + |
| CCTGGGCAGGTTGGTTTCAA | 210 | GGT | xCas9 3.7 (NGT) | −5 | −1 | − |
| AACCTGTCTTGTAACCTTGAAAC | 211 | TTA | FnCpf1 | 21 | 0 | + |
| TCCTTAAACCTGTCTTGTAACCTT | 212 | GAAACC | Nme2Cas9 | 0 | −5 | + |
| TAAACCTGTCTTGTAACCTTGAAA | 213 | CCAACC | Nme2Cas9 | −2 | 1 | + |
| CCTGTCTTGTAACCTTGAAACCAA | 214 | CCTGCC | Nme2Cas9 | −6 | −2 | + |
| CTGTCTTGTAACCTTGAAACCAAC | 215 | CTGCCC | Nme2Cas9 | −7 | −3 | + |
| AGGCCCTGGGCAGGTTGGTTT | 216 | CAAGGT | KKHSauCas9 | −2 | 1 | − |
| IVS2-5 (G>C), | | | | | | |
| HBB:c.315+5G>C | | | | | | |
| AGAACTTCAGGGTGACTCTA | 217 | TGG | Spycas9 | −5 | −1 | + |
| GAACTTCAGGGTGACTCTAT | 218 | GGG | Spycas9 | −6 | −2 | + |
| AACTTCAGGGTGACTCTATG | 219 | GGA | VQRSpyCas9_xCas9-3.7(NGA) | −7 | −3 | + |
| AGCGTCCCATAGAGTCACCC | 220 | TGA | VQRSpyCas9_xCas9-3.7(NGA) | −7 | −3 | − |
| TTCAGGGTGACTCTATGGGA | 221 | CGC | xCas9 3.7 (NGC) | −10 | −6 | + |
| AAACATCAAGCGTCCCATAG | 222 | AGT | xCas9 3.7 (NGT) | 0 | −4 | − |
| GTCCCATAGAGTCACCCTGA | 223 | AGT | xCas9 3.7 (NGT) | −10 | −6 | − |
| AAGAAAACATCAAGCGTCCCA | 224 | TAGAGT | SauCas9 | 0 | −7 | − |
| AGAAAACATCAAGCGTCCCATAGA | 225 | GTCACC | Nme2Cas9 | 0 | −3 | − |
| GAAAACATCAAGCGTCCCATAGAG | 226 | TCACCC | Nme2Cas9 | −1 | 2 | − |
| TCAGGGTGACTCTATGGGACG | 227 | CTTGAT | KKHSauCas9 | −12 | −8 | + |
| AAGCGTCCCATAGAGTCACCC | 228 | TGAAGT | KKHSauCas9 | −7 | −3 | − |
| IVS2-613 (C>T), | | | | | | |
| HBB:c.316-238C>T | | | | | | |
| TTCTTTAGAATGGTACAAAG | 229 | AGG | Spycas9 | −6 | −2 | − |
| GCCTCTTTGTACCATTCTAA | 230 | AGA | VQRSpyCas9_xCas9-3.7(NGA) | −11 | −7 | + |
| TATTCTTTAGAATGGTACAA | 231 | AGA | VQRSpyCas9_xCas9-3.7(NGA) | −4 | 0 | − |
| TAGAATGGTACAAAGAGGCA | 232 | TGA | VQRSpyCas9_xCas9-3.7(NGA) | −11 | −7 | − |
| TCTTTAGAATGGTACAAAGA | 233 | GGC | xCas9 3.7 (NGC) | −7 | −3 | − |
| TACAATGTATCATGCCTCTT | 234 | TGT | xCas9 3.7 (NGT) | 0 | −5 | + |
| ATGCCTCTTTGTACCATTCTA | 235 | AAGAAT | SauCas9 | −10 | −6 | + |
| AATGATACAATGTATCATGCCT | 236 | CTTTGTAC | CjeCas9 | 0 | −8 | + |
| TCTTTAGAATGGTACAAAGAGG | 237 | CATGATAC | CjeCas9 | −9 | −5 | − |
| TTCTTTAGAATGGTACAAAGAGG | 238 | TTA | FnCpf1 | 15 | 4 | − |
| TTTAGAATGGTACAAAGAGGCAT | 239 | TTC | FnCpf1 | 12 | 7 | − |
| ATCACTGTTATTCTTTAGAAT | 240 | GGTACAAA | GeoCas9 | 0 | −6 | − |
| ATGCCTCTTTGTACCATTCT | 241 | AAAGAA | St1Cas9 | −9 | −5 | + |
| ATGCCTCTTTGTACCATTCTAAA | 242 | TATC | Cpf1RVR | 12 | 7 | + |
| ACTGTTATTCTTTAGAATGGTAC | 243 | TATC | Cpf1RVR | 22 | 0 | − |
| ATGATACAATGTATCATGCCTCTT | 244 | TGTACC | Nme2Cas9 | 0 | −5 | + |
| CTTTAGAATGGTACAAAGAGG | 245 | CATGAT | KKHSauCas9 | −9 | −5 | − |
| IVS II-654 (C>T), | | | | | | |
| HBB:c.316-197C>T | | | | | | |
| TATTGCTATTACCTTAACCC | 246 | AGA | VQRSpyCas9_xCas9-3.7(NGA) | −10 | −6 | − |
| AATTTCTGGGTTAAGGTAAT | 247 | AGC | xCas9 3.7 (NGC) | −4 | 0 | + |
| AGTGATAATTTCTGGGTTAA | 248 | GGT | xCas9 3.7 (NGT) | 0 | −5 | + |
| TGGGTTAAGGTAATAGCAATATC | 249 | TTTC | AsCpf1_LbCpf1 | 11 | 8 | + |
| TATGCAGAGATATTGCTATTACC | 250 | TTTA | AsCpf1_LbCpf1 | 21 | 0 | − |
| CTGGGTTAAGGTAATAGCAATAT | 251 | TTT | FnCpf1 | 12 | 7 | + |
| TGGGTTAAGGTAATAGCAATATC | 252 | TTC | FnCpf1 | 11 | 8 | + |
| ATATGCAGAGATATTGCTATTAC | 253 | TTT | FnCpf1 | 22 | 0 | − |
| TATGCAGAGATATTGCTATTACC | 254 | TTA | FnCpf1 | 21 | 0 | − |
| GATATTGCTATTACCTTAAC | 255 | CCAGAA | St1Cas9 | −8 | −4 | − |
| TGCAGAGATATTGCTATTACCTT | 256 | TATA | Cpf1RVR | 19 | 0 | − |
| CAGAGATATTGCTATTACCTTAA | 257 | TATG | Cpf1RVR | 17 | 2 | − |
| ATATTTATATGCAGAGATATTGCT | 258 | ATTACC | Nme2Cas9 | 0 | −6 | − |
| ATATGCAGAGATATTGCTATTACC | 259 | TTAACC | Nme2Cas9 | −3 | 0 | − |
| TATGCAGAGATATTGCTATTACCT | 260 | TAACCC | Nme2Cas9 | −4 | 0 | − |
| TAACAGTGATAATTTCTGGGT | 261 | TAAGGT | KKHSauCas9 | 0 | −8 | + |
| CAGTGATAATTTCTGGGTTAA | 262 | GGTAAT | KKHSauCas9 | 0 | −5 | + |
| TAATTTCTGGGTTAAGGTAAT | 263 | AGCAAT | KKHSauCas9 | −4 | 0 | + |
| ATATTGCTATTACCTTAACCC | 264 | AGAAAT | KKHSauCas9 | −10 | −6 | − |
| IVS II-705 (T>G), | | | | | | |
| HBB:c.316-146T>G | | | | | | |
| CTGCATATAAATTGTAACTG | 265 | AGG | Spycas9 | 0 | −4 | + |
| TAAATTGTAACTGAGGTAAG | 266 | AGG | Spycas9 | −6 | −2 | + |
| TATAAATTGTAACTGAGGTA | 267 | AGA | VQRSpyCas9_xCas9-3.7(NGA) | −4 | 0 | + |
| TGCATATAAATTGTAACTGA | 268 | GGT | xCas9 3.7 (NGT) | 0 | −3 | + |
| AAATTGTAACTGAGGTAAGA | 269 | GGT | xCas9 3.7 (NGT) | −7 | −3 | + |
| AATATGAAACCTCTTACCTC | 270 | AGT | xCas9 3.7 (NGT) | −3 | 0 | − |
| TGCATATAAATTGTAACTGAGGT | 271 | TTTC | AsCpf1_LbCpf1 | 21 | 0 | + |
| CTGCATATAAATTGTAACTGAGG | 272 | TTT | FnCpf1 | 22 | 0 | + |
| TGCATATAAATTGTAACTGAGGT | 273 | TTC | FnCpf1 | 21 | 0 | + |
| GCAATATGAAACCTCTTACCTCA | 274 | TTA | FnCpf1 | 20 | 0 | − |
| AATTGTAACTGAGGTAAGAGGTT | 275 | TATA | Cpf1RVR | 13 | 6 | + |
| AAACCTCTTACCTCAGTTACAAT | 276 | TATG | Cpf1RVR | 12 | 7 | − |
| GCTGCTATTAGCAATATGAAACCT | 277 | CTTACC | Nme2Cas9 | 0 | −8 | − |
| TTTCTGCATATAAATTGTAAC | 278 | TGAGGT | KKHSauCas9 | 0 | −6 | + |
| ATATAAATTGTAACTGAGGTA | 279 | AGAGGT | KKHSauCas9 | −4 | 0 | + |
| TAGCAATATGAAACCTCTTAC | 280 | CTCAGT | KKHSauCas9 | 0 | −3 | − |
| TATGAAACCTCTTACCTCAGT | 281 | TACAAT | KKHSauCas9 | −6 | −2 | − |
| IVS2-726 (A>G), | | | | | | |
| HBB:c.316-125A>G | | | | | | |
| TGTAAGAGGTTTCATATTGC | 282 | TGA | VQRSpyCas9_xCas9-3.7(NGA) | 0 | −4 | + |
| TGTAGCTGCTATCAGCAATA | 283 | TGA | VQRSpyCas9_xCas9-3.7(NGA) | −8 | −4 | − |
| AGAGGTTTCATATTGCTGAT | 284 | AGC | xCas9 3.7 (NGC) | −3 | 0 | + |
| GGTTTCATATTGCTGATAGC | 285 | AGC | xCas9 3.7 (NGC) | −6 | −2 | + |
| GCTGGATTGTAGCTGCTATC | 286 | AGC | xCas9 3.7 (NGC) | −1 | 2 | − |
| TAGCTGCTATCAGCAATATGAAA | 287 | TTG | FnCpf1 | 11 | 8 | − |
| GTTTCATATTGCTGATAGCAGCTA | 288 | CAATCC | Nme2Cas9 | −11 | −7 | + |
| GATTGTAGCTGCTATCAGCAATAT | 289 | GAAACC | Nme2Cas9 | −9 | −5 | − |
| TGATGTAAGAGGTTTCATATT | 290 | GCTGAT | KKHSauCas9 | 0 | −6 | + |
| TTTCATATTGCTGATAGCAGC | 291 | TACAAT | KKHSauCas9 | −9 | −5 | + |
| AGCTGGATTGTAGCTGCTATC | 292 | AGCAAT | KKHSauCas9 | −1 | 2 | − |
| IVS2-745 (C>G), | | | | | | |
| HBB:c.316-106C>G | | | | | | |
| GCTAATAGCAGCTACAATCC | 293 | AGG | Spycas9 | 0 | −5 | + |
| AATAAAAGCAGAATGGTACC | 294 | TGG | Spycas9 | −2 | 1 | − |
| ATAAAAGCAGAATGGTACCT | 295 | GGA | VQRSpyCas9_xCas9-3.7(NGA) | −3 | 0 | − |
| CTACAATCCAGGTACCATTC | 296 | TGC | xCas9 3.7 (NGC) | −9 | −5 | + |
| CAGAATGGTACCTGGATTGT | 297 | AGC | xCas9 3.7 (NGC) | −10 | −6 | − |
| CTAATAGCAGCTACAATCCA | 298 | GGT | xCas9 3.7 (NGT) | 0 | −4 | + |
| AAGCAGAATGGTACCTGGAT | 299 | TGT | xCas9 3.7 (NGT) | −7 | −3 | − |
| CCATAAAATAAAAGCAGAATGGTA | 300 | CCTGGATT | NmeCas9 | 0 | −3 | − |
| AAAATAAAAGCAGAATGGTAC | 301 | CTGGAT | SauCas9 | −1 | 2 | − |
| TATTGCTAATAGCAGCTACAAT | 302 | CCAGGTAC | CjeCas9 | 0 | −7 | + |
| CTAATAGCAGCTACAATCCAGGT | 303 | TTG | FnCpf1 | 22 | 0 | + |
| ATTGCTAATAGCAGCTACAATCCA | 304 | GGTACC | Nme2Cas9 | 0 | −4 | + |
| CCAACCATAAAATAAAAGCAGAAT | 305 | GGTACC | Nme2Cas9 | 0 | −7 | − |
| ATTGCTAATAGCAGCTACAAT | 306 | CCAGGT | KKHSauCas9 | 0 | −7 | + |
| IVS2-761 (A>G), | | | | | | |
| HBB:c.316-90A>G | | | | | | |
| ACCATTCTGCTTTTGTTTTA | 307 | TGG | Spycas9 | −6 | −2 | + |
| TTCTGCTTTTGTTTTATGGT | 308 | TGG | Spycas9 | −10 | −6 | + |
| TCTGCTTTTGTTTTATGGTT | 309 | GGG | Spycas9 | −11 | −7 | + |
| ACCATAAAACAAAAGCAGAA | 310 | TGG | Spycas9 | −11 | −7 | − |
| CTGCTTTTGTTTTATGGTTG | 311 | GGA | VQRSpyCas9_xCas9-3.7(NGA) | −12 | −8 | + |
| CCCAACCATAAAACAAAAGC | 312 | AGA | VQRSpyCas9_xCas9-3.7(NGA) | −7 | −3 | − |
| TATCCCAACCATAAAACAAA | 313 | AGC | xCas9 3.7 (NGC) | −4 | 0 | − |
| TCCAGCTACCATTCTGCTTT | 314 | TGT | xCas9 3.7 (NGT) | 0 | −4 | + |
| CCATTCTGCTTTTGTTTTAT | 315 | GGT | xCas9 3.7 (NGT) | −7 | −3 | + |
| CCATAAAACAAAAGCAGAAT | 316 | GGT | xCas9 3.7 (NGT) | −12 | −8 | − |
| ATTCTGCTTTTGTTTTATGGT | 317 | TGGGAT | SauCas9 | −10 | −6 | + |
| ATCCCAACCATAAAACAAAAG | 318 | CAGAAT | SauCas9 | −6 | −2 | − |
| TCCCAACCATAAAACAAAAGCAG | 319 | TTA | FnCpf1 | 15 | 4 | − |
| ATCCAGCCTTATCCCAACCAT | 320 | AAAACAAA | GeoCas9 | 0 | −7 | − |
| ATCCCAACCATAAAACAAAA | 321 | GCAGAA | St1Cas9 | −5 | −1 | − |
| GCTACCATTCTGCTTTTGTTTTA | 322 | TCCA | Cpf1 RR | 18 | 0 | + |
| GCCTTATCCCAACCATAAAACAA | 323 | TCCA | Cpf1 RR | 21 | 0 | − |
| AACCATAAAACAAAAGCAGAATG | 324 | TCCC | Cpf1 RR | 11 | 8 | − |
| CCAACCATAAAACAAAAGCAGAA | 325 | TATC | Cpf1RVR | 13 | 6 | − |
| GCTACCATTCTGCTTTTGTTT | 326 | TATGGT | KKHSauCas9 | −4 | 0 | + |
| CCAACCATAAAACAAAAGCAG | 327 | AATGGT | KKHSauCas9 | −9 | −5 | − |
| IVS2-781 (C>G), | | | | | | |
| HBB:c.316-70C>G | | | | | | |
| TTATTTTATGGTTGGGATAA | 328 | GGG | Spycas9 | 0 | −5 | + |
| TTTTATGGTTGGGATAAGGG | 329 | TGG | Spycas9 | −1 | 2 | + |
| TTTATGGTTGGGATAAGGGT | 330 | GGA | VQRSpyCas9_xCas9-3.7(NGA) | −2 | 1 | + |
| GGGATAAGGGTGGATTATTC | 331 | TGA | VQRSpyCas9_xCas9-3.7(NGA) | −11 | −7 | + |
| TATTTTATGGTTGGGATAAG | 332 | GGT | xCas9 3.7 (NGT) | 0 | −4 | + |
| CTTTTATTTTATGGTTGGGATAAG | 333 | GGTGGATT | NmeCas9 | 0 | −4 | + |
| CTTTTATTTTATGGTTGGGAT | 334 | AAGGGT | SauCas9 | 0 | −7 | + |
| TATTTTATGGTTGGGATAAGG | 335 | GTGGAT | SauCas9 | 0 | −3 | + |
| TTGGGATAAGGGTGGATTATT | 336 | CTGAGT | SauCas9 | −10 | −6 | + |
| TTTTATGGTTGGGATAAGGGTGG | 337 | TTTA | AsCpf1_LbCpf1 | 20 | 0 | + |
| TGGTTGGGATAAGGGTGGATTAT | 338 | TTTA | AsCpf1_LbCpf1 | 15 | 4 | + |
| TATTTTATGGTTGGGATAAGGGT | 339 | TTT | FnCpf1 | 22 | 0 | + |
| ATTTTATGGTTGGGATAAGGGTG | 340 | TTT | FnCpf1 | 21 | 0 | + |
| TTTTATGGTTGGGATAAGGGTGG | 341 | TTA | FnCpf1 | 20 | 0 | + |
| TATGGTTGGGATAAGGGTGGATT | 342 | TTT | FnCpf1 | 17 | 2 | + |
| ATGGTTGGGATAAGGGTGGATTA | 343 | TTT | FnCpf1 | 16 | 3 | + |
| TGGTTGGGATAAGGGTGGATTAT | 344 | TTA | FnCpf1 | 15 | 4 | + |
| GACTCAGAATAATCCACCCTTAT | 345 | TTG | FnCpf1 | 17 | 2 | − |
| TTATTTTATGGTTGGGATAA | 346 | GGGTG | St3Cas9 | 0 | −5 | + |
| GTTGGGATAAGGGTGGATTATTC | 347 | TATG | Cpf1RVR | 13 | 6 | + |
| GTTGGGATAAGGGTGGATTATTCT | 348 | GAGTCC | Nme2Cas9 | −12 | −8 | + |
| GGGCCTAGCTTGGACTCAGAATAA | 349 | TCCACC | Nme2Cas9 | 0 | −7 | − |
| GGCCTAGCTTGGACTCAGAATAAT | 350 | CCACCC | Nme2Cas9 | 0 | −6 | − |
| GCTTGGACTCAGAATAATCCACCC | 351 | TTATCC | Nme2Cas9 | −3 | 0 | − |
| CTTGGACTCAGAATAATCCACCCT | 352 | TATCCC | Nme2Cas9 | −4 | 0 | − |
| GACTCAGAATAATCCACCCTTATC | 353 | CCAACC | Nme2Cas9 | −8 | −4 | − |
| IVS2-815 (C>T), | | | | | | |
| HBB:c.316-36C>T | | | | | | |
| TTTTATGGTTGGGATAAGGT | 354 | TGG | Spycas9 | −1 | 2 | + |
| TTTATGGTTGGGATAAGGTT | 355 | GGA | VQRSpyCas9_xCas9-3.7(NGA) | −2 | 1 | + |
| GGGATAAGGTTGGATTATTC | 356 | TGA | VQRSpyCas9_xCas9-3.7(NGA) | −11 | −7 | + |
| TTATTTTATGGTTGGGATAA | 357 | GGT | xCas9 3.7 (NGT) | 0 | −5 | + |
| CTTTTATTTTATGGTTGGGATAAG | 358 | GTTGGATT | NmeCas9 | 0 | −4 | + |
| TATTTTATGGTTGGGATAAGG | 359 | TTGGAT | SauCas9 | 0 | −3 | + |
| TTGGGATAAGGTTGGATTATT | 360 | CTGAGT | SauCas9 | −10 | −6 | + |
| TTTTATGGTTGGGATAAGGTTGG | 361 | TTTA | AsCpf1_LbCpf1 | 20 | 0 | + |
| TGGTTGGGATAAGGTTGGATTAT | 362 | TTTA | AsCpf1_LbCpf1 | 15 | 4 | + |
| TATTTTATGGTTGGGATAAGGTT | 363 | TTT | FnCpf1 | 22 | 0 | + |
| ATTTTATGGTTGGGATAAGGTTG | 364 | TTT | FnCpf1 | 21 | 0 | + |
| TTTTATGGTTGGGATAAGGTTGG | 365 | TTA | FnCpf1 | 20 | 0 | + |
| TATGGTTGGGATAAGGTTGGATT | 366 | TTT | FnCpf1 | 17 | 2 | + |
| ATGGTTGGGATAAGGTTGGATTA | 367 | TTT | FnCpf1 | 16 | 3 | + |
| TGGTTGGGATAAGGTTGGATTAT | 368 | TTA | FnCpf1 | 15 | 4 | + |
| GACTCAGAATAATCCAACCTTAT | 369 | TTG | FnCpf1 | 17 | 2 | − |
| GTTGGGATAAGGTTGGATTATTC | 370 | TATG | Cpf1RVR | 13 | 6 | + |
| GTTGGGATAAGGTTGGATTATTCT | 371 | GAGTCC | Nme2Cas9 | −12 | −8 | + |
| GGCCTAGCTTGGACTCAGAATAAT | 372 | CCAACC | Nme2Cas9 | 0 | −6 | − |
| GCTTGGACTCAGAATAATCCAACC | 373 | TTATCC | Nme2Cas9 | −3 | 0 | − |
| CTTGGACTCAGAATAATCCAACCT | 374 | TATCCC | Nme2Cas9 | −4 | 0 | − |
| GACTCAGAATAATCCAACCTTATC | 375 | CCAACC | Nme2Cas9 | −8 | −4 | − |
| GCTTTTATTTTATGGTTGGGA | 376 | TAAGGT | KKHSauCas9 | 0 | −8 | + |
| IVS II-837 (T>G), | | | | | | |
| HBB:c.316-14T>G | | | | | | |
| AGCTGTGGGAGGAAGCTAAG | 377 | AGG | Spycas9 | −5 | −1 | − |
| GGAGCTGTGGGAGGAAGCTA | 378 | AGA | VQRSpyCas9_xCas9-3.7(NGA) | −3 | 0 | − |
| TGGGAGGAAGCTAAGAGGTA | 379 | TGA | VQRSpyCas9_xCas9-3.7(NGA) | −10 | −6 | − |
| TAATCATGTTCATACCTCTT | 380 | AGC | xCas9 3.7 (NGC) | 0 | −4 | + |
| ACCTCTTAGCTTCCTCCCAC | 381 | AGC | xCas9 3.7 (NGC) | −12 | −8 | + |
| GCCCAGGAGCTGTGGGAGGA | 382 | AGC | xCas9 3.7 (NGC) | 0 | −5 | − |
| GCTGTGGGAGGAAGCTAAGA | 383 | GGT | xCas9 3.7 (NGT) | −6 | −2 | − |
| CTAATCATGTTCATACCTCTTAG | 384 | TTTG | AsCpf1_LbCpf1 | 23 | 0 | + |
| CTAATCATGTTCATACCTCTTAG | 385 | TTG | FnCpf1 | 23 | 0 | + |
| ATACCTCTTAGCTTCCTCCCACA | 386 | TTC | FnCpf1 | 11 | 8 | + |
| CCCAGGAGCTGTGGGAGGAAGCT | 387 | TTG | FnCpf1 | 22 | 0 | − |
| TGCTAATCATGTTCATACCTCTTA | 388 | GCTTCC | Nme2Cas9 | 0 | −3 | + |
| TAATCATGTTCATACCTCTTAGCT | 389 | TCCTCC | Nme2Cas9 | −3 | 0 | + |
| AATCATGTTCATACCTCTTAGCTT | 390 | CCTCCC | Nme2Cas9 | −4 | 0 | + |
| TCATACCTCTTAGCTTCCTCCCAC | 391 | AGCTCC | Nme2Cas9 | −12 | −8 | + |
| AGGAGCTGTGGGAGGAAGCTA | 392 | AGAGGT | KKHSauCas9 | −3 | 0 | − |
| IVS2-843 (T>G), | | | | | | |
| HBB:c.316-8T>G | | | | | | |
| TATCTTCCGCCCACAGCTCC | 393 | TGG | Spycas9 | −12 | −8 | + |
| CGTTGCCCAGGAGCTGTGGG | 394 | CGG | Spycas9 | 0 | −3 | − |
| AGCTGTGGGCGGAAGATAAG | 395 | AGG | Spycas9 | −11 | −7 | − |
| CACGTTGCCCAGGAGCTGTG | 396 | GGCG | VRER SpyCas9 | 0 | −5 | − |
| GTTGCCCAGGAGCTGTGGGC | 397 | GGA | VQRSpyCas9_xCas9-3.7(NGA) | −1 | 2 | − |
| GCCCAGGAGCTGTGGGCGGA | 398 | AGA | VQRSpyCas9_xCas9-3.7(NGA) | −4 | 0 | − |
| GGAGCTGTGGGCGGAAGATA | 399 | AGA | VQRSpyCas9_xCas9-3.7(NGA) | −9 | −5 | − |
| TGTTCATACCTCTTATCTTC | 400 | CGC | xCas9 3.7 (NGC) | 0 | −4 | + |
| ACCTCTTATCTTCCGCCCAC | 401 | AGC | xCas9 3.7 (NGC) | −6 | −2 | + |
| CACGTTGCCCAGGAGCTGTG | 402 | GGC | xCas9 3.7 (NGC) | 0 | −5 | − |
| GCTGTGGGCGGAAGATAAGA | 403 | GGT | xCas9 3.7 (NGT) | −12 | −8 | − |
| ATACCTCTTATCTTCCGCCCACA | 404 | TTC | FnCpf1 | 17 | 2 | + |
| CCCAGGAGCTGTGGGCGGAAGAT | 405 | TTG | FnCpf1 | 16 | 3 | − |
| TTGCCCAGGAGCTGTGGGCG | 406 | GAAGAT | St1Cas9 | −2 | 1 | − |
| GCACGTTGCCCAGGAGCTGT | 407 | GGGCG | St3Cas9 | 0 | −6 | − |
| TACCTCTTATCTTCCGCCCACAG | 408 | TTCA | Cpf1 RR | 16 | 3 | + |
| TAATCATGTTCATACCTCTTATCT | 409 | TCCGCC | Nme2Cas9 | 0 | −6 | + |
| AATCATGTTCATACCTCTTATCTT | 410 | CCGCCC | Nme2Cas9 | 0 | −5 | + |
| TCATACCTCTTATCTTCCGCCCAC | 411 | AGCTCC | Nme2Cas9 | −6 | −2 | + |
| GTTGCCCAGGAGCTGTGGGCG | 412 | GAAGAT | KKHSauCas9 | −2 | 1 | − |
| AGGAGCTGTGGGCGGAAGATA | 413 | AGAGGT | KKHSauCas9 | −9 | −5 | − |
| IVS2-844 (C>A), | | | | | | |
| HBB:c.316-7C>A | | | | | | |
| TATCTTCCTACCACAGCTCC | 414 | TGG | Spycas9 | −11 | −7 | + |
| ATCTTCCTACCACAGCTCCT | 415 | GGG | Spycas9 | −12 | −8 | + |
| CGTTGCCCAGGAGCTGTGGT | 416 | AGG | Spycas9 | −1 | 2 | − |
| AGCTGTGGTAGGAAGATAAG | 417 | AGG | Spycas9 | −12 | −8 | − |
| GTTGCCCAGGAGCTGTGGTA | 418 | GGA | VQRSpyCas9_xCas9-3.7(NGA) | −2 | 1 | − |
| GCCCAGGAGCTGTGGTAGGA | 419 | AGA | VQRSpyCas9_xCas9-3.7(NGA) | −5 | −1 | − |
| GGAGCTGTGGTAGGAAGATA | 420 | AGA | VQRSpyCas9_xCas9-3.7(NGA) | −10 | −6 | − |
| ACCTCTTATCTTCCTACCAC | 421 | AGC | xCas9 3.7 (NGC) | −5 | −1 | + |
| GCACGTTGCCCAGGAGCTGT | 422 | GGT | xCas9 3.7 (NGT) | 0 | −5 | − |
| ATACCTCTTATCTTCCTACCACA | 423 | TTC | FnCpf1 | 18 | 0 | + |
| CCCAGGAGCTGTGGTAGGAAGAT | 424 | TTG | FnCpf1 | 15 | 4 | − |
| TTGCCCAGGAGCTGTGGTAG | 425 | GAAGAT | St1Cas9 | −3 | 0 | − |
| TACCTCTTATCTTCCTACCACAG | 426 | TTCA | Cpf1 RR | 17 | 2 | + |
| AATCATGTTCATACCTCTTATCTT | 427 | CCTACC | Nme2Cas9 | 0 | −6 | + |
| TCATACCTCTTATCTTCCTACCAC | 428 | AGCTCC | Nme2Cas9 | −5 | −1 | + |
| ACCAGCACGTTGCCCAGGAGC | 429 | TGTGGT | KKHSauCas9 | 0 | −8 | − |
| GTTGCCCAGGAGCTGTGGTAG | 430 | GAAGAT | KKHSauCas9 | −3 | 0 | − |
| AGGAGCTGTGGTAGGAAGATA | 431 | AGAGGT | KKHSauCas9 | −10 | −6 | − |
| IVS2-844 (C>G), | | | | | | |
| HBB:c.316-7C>G | | | | | | |
| TATCTTCCTGCCACAGCTCC | 432 | TGG | Spycas9 | −11 | −7 | + |
| ATCTTCCTGCCACAGCTCCT | 433 | GGG | Spycas9 | −12 | −8 | + |
| CGTTGCCCAGGAGCTGTGGC | 434 | AGG | Spycas9 | −1 | 2 | − |
| AGCTGTGGCAGGAAGATAAG | 435 | AGG | Spycas9 | −12 | −8 | − |
| GTTGCCCAGGAGCTGTGGCA | 436 | GGA | VQRSpyCas9_xCas9-3.7(NGA) | −2 | 1 | − |
| GCCCAGGAGCTGTGGCAGGA | 437 | AGA | VQRSpyCas9_xCas9-3.7(NGA) | −5 | −1 | − |
| GGAGCTGTGGCAGGAAGATA | 438 | AGA | VQRSpyCas9_xCas9-3.7(NGA) | −10 | −6 | − |
| GTTCATACCTCTTATCTTCC | 439 | TGC | xCas9 3.7 (NGC) | 0 | −4 | + |
| ACCTCTTATCTTCCTGCCAC | 440 | AGC | xCas9 3.7 (NGC) | −5 | −1 | + |
| GCACGTTGCCCAGGAGCTGT | 441 | GGC | xCas9 3.7 (NGC) | 0 | −5 | − |
| ATACCTCTTATCTTCCTGCCACA | 442 | TTC | FnCpf1 | 18 | 0 | + |
| CCCAGGAGCTGTGGCAGGAAGAT | 443 | TTG | FnCpf1 | 15 | 4 | − |
| TTGCCCAGGAGCTGTGGCAG | 444 | GAAGAT | St1Cas9 | −3 | 0 | − |
| TACCTCTTATCTTCCTGCCACAG | 445 | TTCA | Cpf1 RR | 17 | 2 | + |
| AATCATGTTCATACCTCTTATCTT | 446 | CCTGCC | Nme2Cas9 | 0 | −6 | + |
| TCATACCTCTTATCTTCCTGCCAC | 447 | AGCTCC | Nme2Cas9 | −5 | −1 | + |
| GTTGCCCAGGAGCTGTGGCAG | 448 | GAAGAT | KKHSauCas9 | −3 | 0 | − |
| AGGAGCTGTGGCAGGAAGATA | 449 | AGAGGT | KKHSauCas9 | −10 | −6 | − |
| NM_000136.2(FANCC):c. | | | | | | |
| 456+4A>T | | | | | | |
| TTTAAATACACACATTTTTA | 450 | AGC | xCas9 3.7 (NGC) | −11 | −7 | + |
| TCCTGGTTTGCTTAAAAATG | 451 | TGT | xCas9 3.7 (NGT) | 0 | −5 | − |
| CTGGTTTGCTTAAAAATGTG | 452 | TGT | xCas9 3.7 (NGT) | 0 | −3 | − |
| TTTCAAAAGTGATAAATTTTAA | 453 | ATACACAC | CjeCas9 | 0 | −7 | + |
| AAAAGTGATAAATTTTAAATACA | 454 | TTTC | AsCpf1_LbCpf1 | 23 | 0 | + |
| CTTAAAAATGTGTGTATTTAAAA | 455 | TTTG | AsCpf1_LbCpf1 | 13 | 6 | − |
| AAAAGTGATAAATTTTAAATACA | 456 | TTC | FnCpf1 | 23 | 0 | + |
| GCTTAAAAATGTGTGTATTTAAA | 457 | TTT | FnCpf1 | 14 | 5 | − |
| CTTAAAAATGTGTGTATTTAAAA | 458 | TTG | FnCpf1 | 13 | 6 | − |
| AATTTTAAATACACACATTTT | 459 | TAAGCAAA | GeoCas9 | −9 | −5 | + |
| AAAGTGATAAATTTTAAATACAC | 460 | TTCA | Cpf1 RR | 22 | 0 | + |
| CTGGTTTGCTTAAAAATGTGTGT | 461 | TATC | Cpf1RVR | 21 | 0 | − |
| TTGCTTAAAAATGTGTGTATT | 462 | TAAAAT | KKHSauCas9 | −6 | −2 | − |

TABLE 3
β-thalassemia patient HSPC donor genotypes.

| | β-globin | β-globin |
|---|---|---|
| Donor ID | mutation #1 | mutation #2 |
| β⁺β⁰_{#1} | IVS1-110 G > A | Codon 39 (C > T; CAG > TAG) |
| β⁺β⁰_{#2} | IVS1-110 G > A | Codon 39 (C > T; CAG > TAG) |
| β⁺β⁰_{#3} | IVS1-110 G > A | Codon 5 (-CT; CCT −> C--) |
| β⁺β⁺ | IVS1-110 G > A | IVS1-110 G > A |
| β⁺β^{Lepore} | IVS1-110 G > A | Lepore-Boston-Washington deletion |
| β⁺β⁰_{#4} | IVS2-654 C > T | Codon 43 (G > T; GAG > TAG) |
| β⁺β⁰_{#5} | IVS2-654 C > T | Codon 41/42 (--CTTT) |
| β⁺β^{E}_{#1} | IVS2-654 C > T | Codon 26 (G −> A; GAG −> AAG; |
| | | HbE Glu26Lys) |
| β⁺β^{E}_{#2} | IVS2-654 C > T | Codon 26 (G −> A; GAG −> AAG; |
| | | HbE Glu26Lys) |

TABLE 4
Linkage between IVS2-654C > T and rs1609812-T

| | | Genotype at |
|---|---|---|
| Singletons (no.) | IVS2-654C > T | rs1609812 |
| 3 | Homozygous | T/T |
| 19 | Heterozygous | T/T |
| 11 | Heterozygous | T/C |

| | | | Other | Genotype at |
|---|---|---|---|---|
| Family | Relationship | IVS2-654C > T | mutation | rs1609812 |
| #1 | Father | Heterozygous | No | T/T |
| | Mother | No | Codons 41/42 | T/T |
| | Daughter | Heterozygous | Codons 41/42 | T/T |
| #2 | Father | No | Codon 43 | |
| | Mother | Heterozygous | No | T/T |
| | Son | Heterozygous | Codon 43 | T/C |
| #3 | Mother | Heterozygous | No | T/T |
| | Offspring | Heterozygous | No | T/C |
| #4 | Father | No | Codons 41/42 | T/C |
| | Mother | Heterozygous | No | T/C |
| | Offspring | No | No | c/c |
| #5 | Father | Heterozygous | No | T/C |
| | Mother | No | Codon 26 | T/C |
| | Offspring | No | Codon 26 | C/C |
| #6 | Sibling #1 | Heterozygous | No | T/C |
| | Sibling #2 | No | No | T/T |
| | Sibling #3 | Heterozygous | No | T/T |
| #7 | Father | No | Codons 41/42 | T/C |
| | Mother | Heterozygous | No | T/C |
| | Offspring #1 | No | No | C/C |
| | Offspring #2 | No | Codons 41/42 | T/C |

TABLE 5
Oligonucleotides used in Examples

| | | SEQ |
|---|---|---|
| | | ID |
| | Sequence | NO: |
| Primers for | | |
| Sanger analysis. | | |
| IVS1-110_Sanger_F | TGGATGAAGTTGGTGGTGAG | 463 |
| IVS1-110_Sanger_R | AAACATCAAGCGTCCCATAGA | 464 |
| IVS2-654_Sanger_F | TGACCAAATCAGGGTAATTTTGC | 465 |
| IVS2-654_Sanger_R | CAGGAGCTGTGGGAGGAAGA | 466 |
| AAVS1_1F | CACCTTATATTCCCAGGGCCG | 467 |
| AAVS1_1R | CCTAGGACGCACCATTCTCAC | 468 |
| AAVS1_2F | ATTGGGTCTAACCCCCACCT | 469 |
| AAVS1_2R | TCAGTGAAACGCACCAGACA | 470 |
| Primers for | | |
| deep sequencing. | | |
| IVS1-110_deep_3F- | CTCCTGAGGAGAAGTCTGCCGTTAC | 471 |
| HBBsp | | |
| IVS1-110_deep_3R- | GCAGCTCACTCAGTGTGGC | 472 |
| HBBsp | | |
| IVS1-110_deep_1F | TGGGCAGGTTGGTATCAAGG | 473 |
| IVS1-110_deep_1R | GCACTTTCTTGCCATGAGCC | 474 |
| IVS2-654_deep_2F | CTCTTTCTTTCAGGGCAATAATGAT | 475 |
| | AC | |
| IVS2-654_deep_2R | CCAGCCTTATCCCAACCATAAA | 476 |
| Primers | | |
| for RT-PCR. | | |
| HBB-exon1_F | GCAAGGTGAACGTGGATGAAGTT | 477 |
| HBB-exon2_R | GGACAGATCCCCAAAGGACTCAA | 478 |
| HBB-S_qPCR | TGAGGAGAAGTCTGCCGTTAC | 479 |
| HBB_exon3_R | CACCAGCCACCACTTTCTGA | 480 |
| Primers | | |
| for RT-qPCR. | | |
| HBB-S_qPCR | TGAGGAGAAGTCTGCCGTTAC | 481 |
| HBB-AS_qPCR | ACCACCAGCAGCCTGCCCA | 482 |
| HBB_e2-e3 | TTCAGGCTCCTGGGCAAC | 483 |
| R_HBB_exon3 | CACCAGCCACCACTTTCTGA | 484 |
| HBA-S_qPCR | GCCCTGGAGAGGATGTTC | 485 |
| HBA-A_qPCR | TTCTTGCCGTGGCCCTTA | 486 |
| HBG-S_qPCR | GGTTATCAATAAGCTCCTAGTCC | 487 |
| HBG-AS_qPCR | ACAACCAGGAGCCTTCCCA | 488 |
| HBD_RT93_e1_F | GAGGAGAAGACTGCTGTCAATG | 489 |
| HBD_RT93_e2_R | AGGGTAGACCACCAGTAATCTG | 490 |

1. A ribonucleoprotein (RNP) complex comprising a DNA-targeting endonuclease Cas (CRISPR-associated) protein and a guide RNA comprising the sequence of SEQ ID NO: 1 or 3 that targets and hybridizes to a target sequence on a DNA molecule.

2. The RNP complex of claim 1, wherein the CRISPR enzyme is a type II CRISPR system enzyme.

3. The RNP complex of claim 1 or 2, wherein the CRISPR enzyme is a Cas enzyme.

4. The RNP complex of claim 3, wherein the Cas protein is selected from the group consisting of: Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

5. The RNP complex of claim 3, wherein the Cas protein is Cas9 or Cas12a.

6. The RNP complex of any of claims 1-5 for use in altering the genetic sequence of a gene.

7. The RNP complex of claim 6, wherein altering is a nucleotide deletion, insertion or substitution of the genetic sequence.

8. The RNP complex of claim 6, wherein altering promotes proper intron splicing of a gene.

9. The RNP complex of claim 6, wherein altering is correcting a genetic mutation in a gene.

10. The RNP complex of claim 6 or 8, wherein the gene is β-Globin.

11. The RNP complex of claims 8 and 9, wherein the genetic mutation is IVS1-110G>A or IVS2-654C>T.

12. The RNP complex of claims 8 and 9, wherein the genetic mutation is selected from those listed in Table 2.

13. The RNP complex of claim 1, wherein the guide RNA comprises a sequence selected from those listed in Table 2.

14. The RNP complex of any of claims 1-13, further comprising a crRNA/tracrRNA sequence.

15. The RNP complex of any of claims 1-14 for use in an ex vivo method of producing a progenitor cell or a population of progenitor cell wherein the cells or the differentiated progeny thereof have an altered genetic sequence.

16. The RNP complex of any of claims 1-14 for use in an ex vivo method of producing a progenitor cell or a population of progenitor cell wherein the cells or the differentiated progeny thereof have corrected a IVS1-110G>A or IVS2-654C>T mutation.

17. The RNP complex of any of claims 1-14 for use in an ex vivo method of producing a progenitor cell or a population of progenitor cell wherein the cells or the differentiated progeny thereof have at least one genetic modification in the β-Globin gene.

18. The RNP complex of any of claims 1-14 for use in an ex vivo method of producing an isolated genetic engineered human cell or a population of genetic engineered human cells having an altered genetic sequence.

19. The RNP complex of any of claims 1-14 for use in an ex vivo method of producing an isolated genetic engineered human cell or a population of genetic engineered human cells which have corrected a IVS1-110G>A or IVS2-654C>T mutation.

20. The RNP complex of any of claims 1-14 for use in an ex vivo method of producing an isolated genetic engineered human cell or a population of genetic engineered human cells having at least one genetic modification in the β-Globin gene.

21. The RNP complex of any of claims 15-20, wherein the cell is a hematopoietic progenitor cell or a hematopoietic stem cell.

22. The RNP complex of claim 21, wherein the hematopoietic progenitor is a cell of the erythroid lineage.

23. The RNP complex of any of claims 18-20, wherein the isolated human cell is an induced pluripotent stem cell.

24. The RNP complex of claim 16 or 19, wherein IVS1-110G>A or IVS2-654C>T mutation is present in the β-Globin gene

25. A composition comprising the RNP complex of any of claims 1-13.

26. A composition comprising any of the progenitor cell or a population of progenitor cell of claims 15-17, or the isolated genetic engineered human cell or a population of genetic engineered human cells of claims 18-20.

27. The composition of claim 25 or 26, further comprising a pharmaceutically acceptable carrier.

28. The composition of claim 25 for use in an ex vivo method of producing a progenitor cell or a population of progenitor cells wherein the cells or the differentiated progeny therefrom have an altered genetic sequence, have corrected a IVS1-110G>A or IVS2-654C>T mutation, and/or have at least one genetic modification in the β-Globin gene.

29. The composition of claim 25 for use in an ex vivo method of producing an isolated genetic engineered human cell or a population of progenitor cells having an altered genetic sequence, having a corrected a IVS1-110G>A or IVS2-654C>T mutation, and/or having at least one genetic modification in the β-Globin gene.

30. A method for correcting an isolated progenitor cell or a population of isolated progenitor cells having a IVS1-110G>A or IVS2-654C>T mutation in the β-Globin gene, the method comprising contacting an isolated progenitor cell with an effective amount of any of the ribonucleoprotein (RNP) complexes of claims 1-13, or the composition of claim 25, whereby the contacted cells or the differentiated progeny cells therefrom have corrected the IVS1-110G>A or IVS2-654C>T mutation in the β-Globin gene.

31. The method of any one of claims 30, wherein the isolated progenitor cell is a hematopoietic progenitor cell or a hematopoietic stem cell.

32. The method of claim 31, wherein the hematopoietic progenitor is a cell of the erythroid lineage.

33. The method of any one of claims 30, wherein the isolated progenitor cell is an induced pluripotent stem cell.

34. The method of any one of claims 33-33, wherein the isolated progenitor cell is contacted ex vivo or in vitro.

35. A population of genetically edited progenitor cells produced by methods of any of claims 30-34.

36. The population of claim 45, wherein the genetically edited human cells are isolated.

37. A composition comprising isolated genetically edited human cells of claims 35 and 36.

38. The composition of claims 37, further comprising a pharmaceutically acceptable carrier.

39. A method of treating a disease associated with IVS1-110G>A or IVS2-654C>T mutation in the β-Globin gene, the method comprising, administering to a subject in need thereof any of the RNP complexes of any of claims 1-13, any of the compositions of any of claim 25-27 or 37-38, or the population of genetically edited progenitor cells of claims 35-36.

40. The method of claim 39, wherein the disease is thalassemia or β-thalassemia.

41. A ribonucleoprotein (RNP) complex comprising a DNA-targeting endonuclease Cas9 protein and a guide RNA comprising the sequence of SEQ ID NO: 1 that targets and hybridizes to a target sequence on a DNA molecule.

42. A ribonucleoprotein (RNP) complex comprising a DNA-targeting endonuclease Cas12a protein and a guide RNA comprising the sequence of SEQ ID NO: 3 that targets and hybridizes to a target sequence on a DNA molecule.

43. The RNP complex of claim 41, wherein targeting and hybridizing corrects a IVS1-110G>A or mutation is present in the β-Globin gene

44. The RNP complex of claim 42, wherein targeting and hybridizing corrects a IVS2-654C>T mutation is present in the β-Globin gene.


SEQUENCE LISTING (495 sequences)

SEQ ID NO: 1 - RNA, 20 nt, Artificial Sequence
  Description of Artificial Sequence Synthetic oligonucleotide
gggugggaaaauagacuaau

SEQ ID NO: 2 - RNA, 20 nt, Artificial Sequence
  Description of Artificial Sequence Synthetic oligonucleotide
cucccucccaggauccucuc

SEQ ID NO: 3 - RNA, 44 nt, Artificial Sequence
  Description of Artificial Sequence Synthetic oligonucleotide
uaauuucuacuaaguguagauuaugcagaaauauugcuauuacc

SEQ ID NO: 4 - RNA, 44 nt, Artificial Sequence
  Description of Artificial Sequence Synthetic oligonucleotide
uaauuucuacuaaguguagauucuguccccuccaccccacagug

SEQ ID NO: 5 - protein, 7 aa, Simian virus 40
PKKKRKV

SEQ ID NO: 6 - protein, 16 aa, Unknown
  Description of Unknown Nucleoplasmin bipartite NLS sequence
KRPAATKKAGQAKKKK

SEQ ID NO: 7 - protein, 9 aa, Unknown
  Description of Unknown C-myc NLS sequence
PAAKRVKLD

SEQ ID NO: 8 - protein, 11 aa, Unknown
  Description of Unknown C-myc NLS sequence
RQRRNELKRSP

SEQ ID NO: 9 - protein, 38 aa, Homo sapiens
NQSSNFGPMKGGNFGGRSSGPYGGGGQYFAKPRNQGGY

SEQ ID NO: 10 - protein, 42 aa, Unknown
  Description of Unknown IBB domain from importin-alpha sequence
RMRIZFKNKGKDTAELRRRRVEVSVELRKAKKDEQILKRRNV

SEQ ID NO: 11 - protein, 8 aa, Unknown
  Description of Unknown Myoma T protein sequence
VSRKRPRP

SEQ ID NO: 12 - protein, 8 aa, Unknown
  Description of Unknown Myoma T protein sequence
PPKKARED

SEQ ID NO: 13 - protein, 8 aa, Homo sapiens
PQPKKKPL

SEQ ID NO: 14 - protein, 12 aa, Mus musculus
SALIKKKKKMAP

SEQ ID NO: 15 - protein, 5 aa, Influenza virus
DRLRR

SEQ ID NO: 16 - protein, 7 aa, Influenza virus
PKQKKRK

SEQ ID NO: 17 - protein, 10 aa, Hepatitis delta virus
RKLKKKIKKL

SEQ ID NO: 18 - protein, 10 aa, Mus musculus
REKKKFLKRR

SEQ ID NO: 19 - protein, 20 aa, Homo sapiens
KRKGDEVDGVDEVAKKKSKK

SEQ ID NO: 20 - protein, 17 aa, Homo sapiens
RKCLQAGMNLEARKTKK

SEQ ID NO: 21 - protein, 21 aa, Unknown
  Description of Unknown 53BP1 NLS sequence
GKRKLITSEEERSPAKRGRKS

SEQ ID NO: 22 - protein, 6 aa, Unknown
  Description of Unknown BRCA1 NLS sequence
KRKRRP

SEQ ID NO: 23 - protein, 20 aa, Unknown
  Description of Unknown SRC-1 NLS sequence
KRKGSPCDTLASSTEKRRRE

SEQ ID NO: 24 - protein, 12 aa, Unknown
  Description of Unknown IRF3 NLS sequence
KRNFRSALNRKE

SEQ ID NO: 25 - protein, 4 aa, Artificial Sequence
  Description of Artificial Sequence Synthetic peptide
GGSG

SEQ ID NO: 26 - protein, 12 aa, Artificial Sequence
  Description of Artificial Sequence Synthetic peptide
GGSGGGSGGGSG

SEQ ID NO: 27 - protein, 20 aa, Artificial Sequence
  Description of Artificial Sequence Synthetic peptide
GGSGGGSGGGSGGGSGGGSG

SEQ ID NO: 28 - protein, 15 aa, Artificial Sequence
  Description of Artificial Sequence Synthetic peptide
TGGGPGGGAAAGSGS

SEQ ID NO: 29 - DNA, 12 bp, Artificial Sequence
  Description of Artificial Sequence Synthetic oligonucleotide
ggcggtagcggc

SEQ ID NO: 30 - DNA, 36 bp, Artificial Sequence
  Description of Artificial Sequence Synthetic oligonucleotide
ggcggtagcggcggaggcagcggtggcggcagcggc

SEQ ID NO: 31 - DNA, 60 bp, Artificial Sequence
  Description of Artificial Sequence Synthetic oligonucleotide
ggcggtagcggcggcggtagcggcggaggcagcggtggcggcagcggcggcggtagcggc

SEQ ID NO: 32 - DNA, 45 bp, Artificial Sequence
  Description of Artificial Sequence Synthetic oligonucleotide
accggtggtggtcccgggggtggtgcggccgcaggcagcggaagc

SEQ ID NO: 33 - protein, 32 aa, Artificial Sequence
  Description of Artificial Sequence Synthetic polypeptide
SGGSSGGSSGSETPGTSESATPESSGGSSGGS

SEQ ID NO: 34 - DNA, 96 bp, Artificial Sequence
  Description of Artificial Sequence Synthetic oligonucleotide
tctggaggatctagcggaggatcctctggaagcgagacaccaggcacaagcgagtccgccacaccagagagctccggcggctcctccggaggatcc

SEQ ID NO: 35 - DNA, 20 bp, Artificial Sequence
  Description of Artificial Sequence Synthetic oligonucleotide
gggtgggaaaatagactaat

SEQ ID NO: 36 - DNA, 20 bp, Artificial Sequence
  Description of Artificial Sequence Synthetic oligonucleotide
gggaaaatagactaataggc

SEQ ID NO: 37 - DNA, 20 bp, Artificial Sequence
  Description of Artificial Sequence Synthetic oligonucleotide
gaaaatagactaataggcag

SEQ ID NO: 38 - DNA, 20 bp, Artificial Sequence
  Description of Artificial Sequence Synthetic oligonucleotide
aaatagactaataggcagag

SEQ ID NO: 39 - DNA, 20 bp, Artificial Sequence
  Description of Artificial Sequence Synthetic oligonucleotide
ggtgggaaaatagactaata

SEQ ID NO: 40 - DNA, 20 bp, Artificial Sequence
  Description of Artificial Sequence Synthetic oligonucleotide
actgactctctctgcctatt

SEQ ID NO: 41 - DNA, 21 bp, Artificial Sequence
  Description of Artificial Sequence Synthetic oligonucleotide
gaaaatagactaataggcaga

SEQ ID NO: 42 - DNA, 24 bp, Artificial Sequence
  Description of Artificial Sequence Synthetic oligonucleotide
tgactctctctgcctattagtcta

SEQ ID NO: 43 - DNA, 24 bp, Artificial Sequence
  Description of Artificial Sequence Synthetic oligonucleotide
gactctctctgcctattagtctat

SEQ ID NO: 44 - DNA, 24 bp, Artificial Sequence
  Description of Artificial Sequence Synthetic oligonucleotide
tctctctgcctattagtctatttt

SEQ ID NO: 45 - DNA, 24 bp, Artificial Sequence
  Description of Artificial Sequence Synthetic oligonucleotide
ctctctgcctattagtctattttc

SEQ ID NO: 46 - DNA, 21 bp, Artificial Sequence
  Description of Artificial Sequence Synthetic oligonucleotide
aggcactgactctctctgcct

SEQ ID NO: 47 - DNA, 21 bp, Artificial Sequence
  Description of Artificial Sequence Synthetic oligonucleotide
agcctaagggtgggaaaatag

SEQ ID NO: 48 - DNA, 20 bp, Artificial Sequence
  Description of Artificial Sequence Synthetic oligonucleotide
gggtgggaaactagaccaat

SEQ ID NO: 49 - DNA, 20 bp, Artificial Sequence
  Description of Artificial Sequence Synthetic oligonucleotide
cagcctaagggtgggaaact

SEQ ID NO: 50 - DNA, 20 bp, Artificial Sequence
  Description of Artificial Sequence Synthetic oligonucleotide
ggtgggaaactagaccaata

SEQ ID NO: 51 - DNA, 20 bp, Artificial Sequence
  Description of Artificial Sequence Synthetic oligonucleotide
ctctctctgcctattggtct

SEQ ID NO: 52 - DNA, 24 bp, Artificial Sequence
  Description of Artificial Sequence Synthetic oligonucleotide
tgactctctctgcctattggtcta

SEQ ID NO: 53 - DNA, 24 bp, Artificial Sequence
  Description of Artificial Sequence Synthetic oligonucleotide
gactctctctgcctattggtctag

SEQ ID NO: 54 - DNA, 24 bp, Artificial Sequence
  Description of Artificial Sequence Synthetic oligonucleotide
tctctctgcctattggtctagttt

SEQ ID NO: 55 - DNA, 24 bp, Artificial Sequence
  Description of Artificial Sequence Synthetic oligonucleotide
ctctctgcctattggtctagtttc

SEQ ID NO: 56 - DNA, 24 bp, Artificial Sequence
  Description of Artificial Sequence Synthetic oligonucleotide
accagcagcctaagggtgggaaac

SEQ ID NO: 57 - DNA, 21 bp, Artificial Sequence
  Description of Artificial Sequence Synthetic oligonucleotide
ctgactctctctgcctattgg

SEQ ID NO: 58 - DNA, 21 bp, Artificial Sequence
  Description of Artificial Sequence Synthetic oligonucleotide
agcctaagggtgggaaactag

SEQ ID NO: 59 - DNA, 20 bp, Artificial Sequence
  Description of Artificial Sequence Synthetic oligonucleotide
cactgactctctctgcctat

SEQ ID NO: 60 - DNA, 20 bp, Artificial Sequence
  Description of Artificial Sequence Synthetic oligonucleotide
gggtgggaaaatagacctat

SEQ ID NO: 61 - DNA, 20 bp, Artificial Sequence
  Description of Artificial Sequence Synthetic oligonucleotide
gggaaaatagacctataggc

SEQ ID NO: 62 - DNA, 20 bp, Artificial Sequence
  Description of Artificial Sequence Synthetic oligonucleotide
gaaaatagacctataggcag

SEQ ID NO: 63 - DNA, 20 bp, Artificial Sequence
  Description of Artificial Sequence Synthetic oligonucleotide
aaatagacctataggcagag

SEQ ID NO: 64 - DNA, 20 bp, Artificial Sequence
  Description of Artificial Sequence Synthetic oligonucleotide
ggtgggaaaatagacctata

SEQ ID NO: 65 - DNA, 20 bp, Artificial Sequence
  Description of Artificial Sequence Synthetic oligonucleotide
actgactctctctgcctata

SEQ ID NO: 66 - DNA, 21 bp, Artificial Sequence
  Description of Artificial Sequence Synthetic oligonucleotide
gaaaatagacctataggcaga

SEQ ID NO: 67 - DNA, 24 bp, Artificial Sequence
  Description of Artificial Sequence Synthetic oligonucleotide
tgactctctctgcctataggtcta

SEQ ID NO: 68 - DNA, 24 bp, Artificial Sequence
  Description of Artificial Sequence Synthetic oligonucleotide
gactctctctgcctataggtctat

SEQ ID NO: 69 - DNA, 24 bp, Artificial Sequence
  Description of Artificial Sequence Synthetic oligonucleotide
tctctctgcctataggtctatttt

SEQ ID NO: 70 - DNA, 24 bp, Artificial Sequence
  Description of Artificial Sequence Synthetic oligonucleotide
ctctctgcctataggtctattttc

SEQ ID NO: 71 - DNA, 21 bp, Artificial Sequence
  Description of Artificial Sequence Synthetic oligonucleotide
aggcactgactctctctgcct

SEQ ID NO: 72 - DNA, 20 bp, Artificial Sequence
  Description of Artificial Sequence Synthetic oligonucleotide
ccctgggcaggttgttatca

SEQ ID NO: 73 - DNA, 20 bp, Artificial Sequence
  Description of Artificial Sequence Synthetic oligonucleotide
accttgataacaacctgccc

SEQ ID NO: 74 - DNA, 20 bp, Artificial Sequence
  Description of Artificial Sequence Synthetic oligonucleotide
ccttgataacaacctgccca

SEQ ID NO: 75 - DNA, 20 bp, Artificial Sequence
  Description of Artificial Sequence Synthetic oligonucleotide
ttgtaaccttgataacaacc

SEQ ID NO: 76 - DNA, 20 bp, Artificial Sequence
  Description of Artificial Sequence Synthetic oligonucleotide
tggtgaggccctgggcaggt

SEQ ID NO: 77 - DNA, 20 bp, Artificial Sequence
  Description of Artificial Sequence Synthetic oligonucleotide
cctgggcaggttgttatcaa

SEQ ID NO: 78 - DNA, 23 bp, Artificial Sequence
  Description of Artificial Sequence Synthetic oligonucleotide
aacctgtcttgtaaccttgataa

SEQ ID NO: 79 - DNA, 23 bp, Artificial Sequence
  Description of Artificial Sequence Synthetic oligonucleotide
taaccttgataacaacctgccca

SEQ ID NO: 80 - DNA, 24 bp, Artificial Sequence
  Description of Artificial Sequence Synthetic oligonucleotide
taaacctgtcttgtaaccttgata

SEQ ID NO: 81 - DNA, 24 bp, Artificial Sequence
  Description of Artificial Sequence Synthetic oligonucleotide
cctgtcttgtaaccttgataacaa

SEQ ID NO: 82 - DNA, 24 bp, Artificial Sequence
  Description of Artificial Sequence Synthetic oligonucleotide
ctgtcttgtaaccttgataacaac

SEQ ID NO: 83 - DNA, 24 bp, Artificial Sequence
  Description of Artificial Sequence Synthetic oligonucleotide
tgtaaccttgataacaacctgccc

SEQ ID NO: 84 - DNA, 21 bp, Artificial Sequence
  Description of Artificial Sequence Synthetic oligonucleotide
aggccctgggcaggttgttat

SEQ ID NO: 85 - DNA, 20 bp, Artificial Sequence
  Description of Artificial Sequence Synthetic oligonucleotide
ccctgggcaggttgctatca

SEQ ID NO: 86 - DNA, 20 bp, Artificial Sequence
  Description of Artificial Sequence Synthetic oligonucleotide
accttgatagcaacctgccc

SEQ ID NO: 87 - DNA, 20 bp, Artificial Sequence
  Description of Artificial Sequence Synthetic oligonucleotide
ccttgatagcaacctgccca

SEQ ID NO: 88 - DNA, 20 bp, Artificial Sequence
  Description of Artificial Sequence Synthetic oligonucleotide
tggtgaggccctgggcaggt

SEQ ID NO: 89 - DNA, 20 bp, Artificial Sequence
  Description of Artificial Sequence Synthetic oligonucleotide
acctgtcttgtaaccttgat

SEQ ID NO: 90 - DNA, 20 bp, Artificial Sequence
  Description of Artificial Sequence Synthetic oligonucleotide
ttgtaaccttgatagcaacc

SEQ ID NO: 91 - DNA, 20 bp, Artificial Sequence
  Description of Artificial Sequence Synthetic oligonucleotide
cctgggcaggttgctatcaa

SEQ ID NO: 92 - DNA, 23 bp, Artificial Sequence
  Description of Artificial Sequence Synthetic oligonucleotide
aacctgtcttgtaaccttgatag

SEQ ID NO: 93 - DNA, 23 bp, Artificial Sequence
  Description of Artificial Sequence Synthetic oligonucleotide
taaccttgatagcaacctgccca

SEQ ID NO: 94 - DNA, 24 bp, Artificial Sequence
  Description of Artificial Sequence Synthetic oligonucleotide
taaacctgtcttgtaaccttgata

SEQ ID NO: 95 - DNA, 24 bp, Artificial Sequence
  Description of Artificial Sequence Synthetic oligonucleotide
cctgtcttgtaaccttgatagcaa

SEQ ID NO: 96 - DNA, 24 bp, Artificial Sequence
  Description of Artificial Sequence Synthetic oligonucleotide
ctgtcttgtaaccttgatagcaac

SEQ ID NO: 97 - DNA, 24 bp, Artificial Sequence
  Description of Artificial Sequence Synthetic oligonucleotide
tgtaaccttgatagcaacctgccc

SEQ ID NO: 98 - DNA, 21 bp, Artificial Sequence
  Description of Artificial Sequence Synthetic oligonucleotide
aggccctgggcaggttgctat

SEQ ID NO: 99 - DNA, 20 bp, Artificial Sequence
  Description of Artificial Sequence Synthetic oligonucleotide
ccctgggcaggttgttatca

SEQ ID NO: 100 - DNA, 20 bp, Artificial Sequence
  Description of Artificial Sequence Synthetic oligonucleotide
accttgataacaacctgccc

SEQ ID NO: 101 - DNA, 20 bp, Artificial Sequence
  Description of Artificial Sequence Synthetic oligonucleotide
ccttgataacaacctgccca

SEQ ID NO: 102 - DNA, 20 bp, Artificial Sequence
  Description of Artificial Sequence Synthetic oligonucleotide
ttgtaaccttgataacaacc

SEQ ID NO: 103 - DNA, 20 bp, Artificial Sequence
  Description of Artificial Sequence Synthetic oligonucleotide
tggtgaggccctgggcaggt

SEQ ID NO: 104 - DNA, 20 bp, Artificial Sequence
  Description of Artificial Sequence Synthetic oligonucleotide
cctgggcaggttgttatcaa

SEQ ID NO: 105 - DNA, 23 bp, Artificial Sequence
  Description of Artificial Sequence Synthetic oligonucleotide
aacctgtcttgtaaccttgataa

SEQ ID NO: 106 - DNA, 23 bp, Artificial Sequence
  Description of Artificial Sequence Synthetic oligonucleotide
taaccttgataacaacctgccca

SEQ ID NO: 107 - DNA, 24 bp, Artificial Sequence
  Description of Artificial Sequence Synthetic oligonucleotide
taaacctgtcttgtaaccttgata

SEQ ID NO: 108 - DNA, 24 bp, Artificial Sequence
  Description of Artificial Sequence Synthetic oligonucleotide
cctgtcttgtaaccttgataacaa

SEQ ID NO: 109 - DNA, 24 bp, Artificial Sequence
  Description of Artificial Sequence Synthetic oligonucleotide
ctgtcttgtaaccttgataacaac

SEQ ID NO: 110 - DNA, 24 bp, Artificial Sequence
  Description of Artificial Sequence Synthetic oligonucleotide
tgtaaccttgataacaacctgccc

SEQ ID NO: 111 - DNA, 21 bp, Artificial Sequence
  Description of Artificial Sequence Synthetic oligonucleotide
aggccctgggcaggttgttat

SEQ ID NO: 112 - DNA, 20 bp, Artificial Sequence
  Description of Artificial Sequence Synthetic oligonucleotide
ccctgggcaggttgttatca

SEQ ID NO: 113 - DNA, 20 bp, Artificial Sequence
  Description of Artificial Sequence Synthetic oligonucleotide
accttgataacaacctgccc

SEQ ID NO: 114 - DNA, 20 bp, Artificial Sequence
  Description of Artificial Sequence Synthetic oligonucleotide
ccttgataacaacctgccca

SEQ ID NO: 115 - DNA, 20 bp, Artificial Sequence
  Description of Artificial Sequence Synthetic oligonucleotide
ttgtaaccttgataacaacc

SEQ ID NO: 116 - DNA, 20 bp, Artificial Sequence
  Description of Artificial Sequence Synthetic oligonucleotide
tggtgaggccctgggcaggt

SEQ ID NO: 117 - DNA, 20 bp, Artificial Sequence
  Description of Artificial Sequence Synthetic oligonucleotide
cctgggcaggttgttatcaa

SEQ ID NO: 118 - DNA, 23 bp, Artificial Sequence
  Description of Artificial Sequence Synthetic oligonucleotide
aacctgtcttgtaaccttgataa

SEQ ID NO: 119 - DNA, 23 bp, Artificial Sequence
  Description of Artificial Sequence Synthetic oligonucleotide
taaccttgataacaacctgccca

SEQ ID NO: 120 - DNA, 24 bp, Artificial Sequence
  Description of Artificial Sequence Synthetic oligonucleotide
taaacctgtcttgtaaccttgata

SEQ ID NO: 121 - DNA, 24 bp, Artificial Sequence
  Description of Artificial Sequence Synthetic oligonucleotide
cctgtcttgtaaccttgataacaa

SEQ ID NO: 122 - DNA, 24 bp, Artificial Sequence
  Description of Artificial Sequence Synthetic oligonucleotide
ctgtcttgtaaccttgataacaac

SEQ ID NO: 123 - DNA, 24 bp, Artificial Sequence
  Description of Artificial Sequence Synthetic oligonucleotide
tgtaaccttgataacaacctgccc

SEQ ID NO: 124 - DNA, 21 bp, Artificial Sequence
  Description of Artificial Sequence Synthetic oligonucleotide
aggccctgggcaggttgttat

SEQ ID NO: 125 - DNA, 20 bp, Artificial Sequence
  Description of Artificial Sequence Synthetic oligonucleotide
gtgcggaggccctggagagg

SEQ ID NO: 126 - DNA, 20 bp, Artificial Sequence
  Description of Artificial Sequence Synthetic oligonucleotide
tgcggaggccctggagaggt

SEQ ID NO: 127 - DNA, 20 bp, Artificial Sequence
  Description of Artificial Sequence Synthetic oligonucleotide
gcggaggccctggagaggtg

SEQ ID NO: 128 - DNA, 20 bp, Artificial Sequence
  Description of Artificial Sequence Synthetic oligonucleotide
ggagggagccccacctctcc

SEQ ID NO: 129 - DNA, 20 bp, Artificial Sequence
  Description of Artificial Sequence Synthetic oligonucleotide
gagggagccccacctctcca

SEQ ID NO: 130 - DNA, 20 bp, Artificial Sequence
  Description of Artificial Sequence Synthetic oligonucleotide
cggaggccctggagaggtgg

SEQ ID NO: 131 - DNA, 20 bp, Artificial Sequence
  Description of Artificial Sequence Synthetic oligonucleotide
agggagccccacctctccag

SEQ ID NO: 132 - DNA, 20 bp, Artificial Sequence
  Description of Artificial Sequence Synthetic oligonucleotide
gtgcggaggccctggagagg

SEQ ID NO: 133 - DNA, 23 bp, Artificial Sequence
  Description of Artificial Sequence Synthetic oligonucleotide
gtgcggaggccctggagaggtgg

SEQ ID NO: 134 - DNA, 24 bp, Artificial Sequence
  Description of Artificial Sequence Synthetic oligonucleotide
ggtgcggaggccctggagaggtgg

SEQ ID NO: 135 - DNA, 24 bp, Artificial Sequence
  Description of Artificial Sequence Synthetic oligonucleotide
gtgcggaggccctggagaggtggg

SEQ ID NO: 136 - DNA, 24 bp, Artificial Sequence
  Description of Artificial Sequence Synthetic oligonucleotide
cggaggccctggagaggtggggct

SEQ ID NO: 137 - DNA, 24 bp, Artificial Sequence
  Description of Artificial Sequence Synthetic oligonucleotide
ggaggccctggagaggtggggctc

SEQ ID NO: 138 - DNA, 24 bp, Artificial Sequence
  Description of Artificial Sequence Synthetic oligonucleotide
gaggccctggagaggtggggctcc

SEQ ID NO: 139 - DNA, 24 bp, Artificial Sequence
  Description of Artificial Sequence Synthetic oligonucleotide
gagcccgggtcggagcaggggagg

SEQ ID NO: 140 - DNA, 24 bp, Artificial Sequence
  Description of Artificial Sequence Synthetic oligonucleotide
agcccgggtcggagcaggggaggg

SEQ ID NO: 141 - DNA, 24 bp, Artificial Sequence
  Description of Artificial Sequence Synthetic oligonucleotide
ccgggtcggagcaggggagggagc

SEQ ID NO: 142 - DNA, 24 bp, Artificial Sequence
  Description of Artificial Sequence Synthetic oligonucleotide
tcggagcaggggagggagccccac

SEQ ID NO: 143 - DNA, 24 bp, Artificial Sequence
  Description of Artificial Sequence Synthetic oligonucleotide
caggggagggagccccacctctcc

SEQ ID NO: 144 - DNA, 20 bp, Artificial Sequence
  Description of Artificial Sequence Synthetic oligonucleotide
ggacggttgagggtggtctg

SEQ ID NO: 145 - DNA, 20 bp, Artificial Sequence
  Description of Artificial Sequence Synthetic oligonucleotide
gacggttgagggtggtctgt

SEQ ID NO: 146 - DNA, 20 bp, Artificial Sequence
  Description of Artificial Sequence Synthetic oligonucleotide
ttgagggtggtctgtgggtc

SEQ ID NO: 147 - DNA, 20 bp, Artificial Sequence
  Description of Artificial Sequence Synthetic oligonucleotide
tgagggtggtctgtgggtcc

SEQ ID NO: 148 - DNA, 20 bp, Artificial Sequence
  Description of Artificial Sequence Synthetic oligonucleotide
gagggtggtctgtgggtccg

SEQ ID NO: 149 - DNA, 20 bp, Artificial Sequence
  Description of Artificial Sequence Synthetic oligonucleotide
cctcgcccgcccggacccac

SEQ ID NO: 150 - DNA, 20 bp, Artificial Sequence
  Description of Artificial Sequence Synthetic oligonucleotide
gagggtggtctgtgggtccg

SEQ ID NO: 151 - DNA, 20 bp, Artificial Sequence
  Description of Artificial Sequence Synthetic oligonucleotide
acccacagaccaccctcaac

SEQ ID NO: 152 - DNA, 20 bp, Artificial Sequence
  Description of Artificial Sequence Synthetic oligonucleotide
gggccaggacggttgagggt

SEQ ID NO: 153 - DNA, 20 bp, Artificial Sequence
  Description of Artificial Sequence Synthetic oligonucleotide
caggacggttgagggtggtc

SEQ ID NO: 154 - DNA, 20 bp, Artificial Sequence
  Description of Artificial Sequence Synthetic oligonucleotide
acggttgagggtggtctgtg

SEQ ID NO: 155 - DNA, 21 bp, Artificial Sequence
  Description of Artificial Sequence Synthetic oligonucleotide
caggacggttgagggtggtct

SEQ ID NO: 156 - DNA, 20 bp, Artificial Sequence
  Description of Artificial Sequence Synthetic oligonucleotide
tgagggtggtctgtgggtcc

SEQ ID NO: 157 - DNA, 23 bp, Artificial Sequence
  Description of Artificial Sequence Synthetic oligonucleotide
gggccaggacggttgagggtggt

SEQ ID NO: 158 - DNA, 24 bp, Artificial Sequence
  Description of Artificial Sequence Synthetic oligonucleotide
gggctcctcgcccgcccggaccca

SEQ ID NO: 159 - DNA, 24 bp, Artificial Sequence
  Description of Artificial Sequence Synthetic oligonucleotide
ctcctcgcccgcccggacccacag

SEQ ID NO: 160 - DNA, 24 bp, Artificial Sequence
  Description of Artificial Sequence Synthetic oligonucleotide
tcctcgcccgcccggacccacaga

SEQ ID NO: 161 - DNA, 24 bp, Artificial Sequence
  Description of Artificial Sequence Synthetic oligonucleotide
cccgcccggacccacagaccaccc

SEQ ID NO: 162 - DNA, 24 bp, Artificial Sequence
  Description of Artificial Sequence Synthetic oligonucleotide
cccggacccacagaccaccctcaa

SEQ ID NO: 163 - DNA, 24 bp, Artificial Sequence
  Description of Artificial Sequence Synthetic oligonucleotide
ccaggacggttgagggtggtctgt

SEQ ID NO: 164 - DNA, 21 bp, Artificial Sequence
  Description of Artificial Sequence Synthetic oligonucleotide
tccggggccaggacggttgag

SEQ ID NO: 165 - DNA, 20 bp, Artificial Sequence
  Description of Artificial Sequence Synthetic oligonucleotide
ggacggttgagggtggtctg

SEQ ID NO: 166 - DNA, 20 bp, Artificial Sequence
  Description of Artificial Sequence Synthetic oligonucleotide
gacggttgagggtggtctgt

SEQ ID NO: 167 - DNA, 20 bp, Artificial Sequence
  Description of Artificial Sequence Synthetic oligonucleotide
ttgagggtggtctgtgggtc

SEQ ID NO: 168 - DNA, 20 bp, Artificial Sequence
  Description of Artificial Sequence Synthetic oligonucleotide
tgagggtggtctgtgggtcc

SEQ ID NO: 169 - DNA, 20 bp, Artificial Sequence
  Description of Artificial Sequence Synthetic oligonucleotide
gagggtggtctgtgggtccg

SEQ ID NO: 170 - DNA, 20 bp, Artificial Sequence
  Description of Artificial Sequence Synthetic oligonucleotide
cctcgcccgcccggacccac

SEQ ID NO: 171 - DNA, 20 bp, Artificial Sequence
  Description of Artificial Sequence Synthetic oligonucleotide
gagggtggtctgtgggtccg

SEQ ID NO: 172 - DNA, 20 bp, Artificial Sequence
  Description of Artificial Sequence Synthetic oligonucleotide
acccacagaccaccctcaac

SEQ ID NO: 173 - DNA, 20 bp, Artificial Sequence
  Description of Artificial Sequence Synthetic oligonucleotide
gggccaggacggttgagggt

SEQ ID NO: 174 - DNA, 20 bp, Artificial Sequence
  Description of Artificial Sequence Synthetic oligonucleotide
caggacggttgagggtggtc

SEQ ID NO: 175 - DNA, 20 bp, Artificial Sequence
  Description of Artificial Sequence Synthetic oligonucleotide
acggttgagggtggtctgtg

SEQ ID NO: 176 - DNA, 21 bp, Artificial Sequence
  Description of Artificial Sequence Synthetic oligonucleotide
caggacggttgagggtggtct

SEQ ID NO: 177 - DNA, 20 bp, Artificial Sequence
  Description of Artificial Sequence Synthetic oligonucleotide
tgagggtggtctgtgggtcc

SEQ ID NO: 178 - DNA, 23 bp, Artificial Sequence
  Description of Artificial Sequence Synthetic oligonucleotide
gggccaggacggttgagggtggt

SEQ ID NO: 179 - DNA, 24 bp, Artificial Sequence
  Description of Artificial Sequence Synthetic oligonucleotide
gggctcctcgcccgcccggaccca

SEQ ID NO: 180 - DNA, 24 bp, Artificial Sequence
  Description of Artificial Sequence Synthetic oligonucleotide
ctcctcgcccgcccggacccacag

SEQ ID NO: 181 - DNA, 24 bp, Artificial Sequence
  Description of Artificial Sequence Synthetic oligonucleotide
tcctcgcccgcccggacccacaga

SEQ ID NO: 182 - DNA, 24 bp, Artificial Sequence
  Description of Artificial Sequence Synthetic oligonucleotide
cccgcccggacccacagaccaccc

SEQ ID NO: 183 - DNA, 24 bp, Artificial Sequence
  Description of Artificial Sequence Synthetic oligonucleotide
cccggacccacagaccaccctcaa

SEQ ID NO: 184 - DNA, 24 bp, Artificial Sequence
  Description of Artificial Sequence Synthetic oligonucleotide
ccaggacggttgagggtggtctgt

SEQ ID NO: 185 - DNA, 21 bp, Artificial Sequence
  Description of Artificial Sequence Synthetic oligonucleotide
tccggggccaggacggttgag

SEQ ID NO: 186 - DNA, 20 bp, Artificial Sequence
  Description of Artificial Sequence Synthetic oligonucleotide
ggacggttgagggtggtctg

SEQ ID NO: 187 - DNA, 20 bp, Artificial Sequence
  Description of Artificial Sequence Synthetic oligonucleotide
gacggttgagggtggtctgt

SEQ ID NO: 188 - DNA, 20 bp, Artificial Sequence
  Description of Artificial Sequence Synthetic oligonucleotide
ttgagggtggtctgtgggtc

SEQ ID NO: 189 - DNA, 20 bp, Artificial Sequence
  Description of Artificial Sequence Synthetic oligonucleotide
tgagggtggtctgtgggtcc

SEQ ID NO: 190 - DNA, 20 bp, Artificial Sequence
  Description of Artificial Sequence Synthetic oligonucleotide
gagggtggtctgtgggtccg

SEQ ID NO: 191 - DNA, 20 bp, Artificial Sequence
  Description of Artificial Sequence Synthetic oligonucleotide
cctcgcccgcccggacccac

SEQ ID NO: 192 - DNA, 20 bp, Artificial Sequence
  Description of Artificial Sequence Synthetic oligonucleotide
gagggtggtctgtgggtccg

SEQ ID NO: 193 - DNA, 20 bp, Artificial Sequence
  Description of Artificial Sequence Synthetic oligonucleotide
acccacagaccaccctcaac

SEQ ID NO: 194 - DNA, 20 bp, Artificial Sequence
  Description of Artificial Sequence Synthetic oligonucleotide
gggccaggacggttgagggt

SEQ ID NO: 195 - DNA, 20 bp, Artificial Sequence
  Description of Artificial Sequence Synthetic oligonucleotide
caggacggttgagggtggtc

SEQ ID NO: 196 - DNA, 20 bp, Artificial Sequence
  Description of Artificial Sequence Synthetic oligonucleotide
acggttgagggtggtctgtg

SEQ ID NO: 197 - DNA, 21 bp, Artificial Sequence
  Description of Artificial Sequence Synthetic oligonucleotide
caggacggttgagggtggtct

SEQ ID NO: 198 - DNA, 20 bp, Artificial Sequence
  Description of Artificial Sequence Synthetic oligonucleotide
tgagggtggtctgtgggtcc

SEQ ID NO: 199 - DNA, 23 bp, Artificial Sequence
  Description of Artificial Sequence Synthetic oligonucleotide
gggccaggacggttgagggtggt

SEQ ID NO: 200 - DNA, 24 bp, Artificial Sequence
  Description of Artificial Sequence Synthetic oligonucleotide
gggctcctcgcccgcccggaccca

SEQ ID NO: 201 - DNA, 24 bp, Artificial Sequence
  Description of Artificial Sequence Synthetic oligonucleotide
ctcctcgcccgcccggacccacag

SEQ ID NO: 202 - DNA, 24 bp, Artificial Sequence
  Description of Artificial Sequence Synthetic oligonucleotide
tcctcgcccgcccggacccacaga

SEQ ID NO: 203 - DNA, 24 bp, Artificial Sequence
  Description of Artificial Sequence Synthetic oligonucleotide
cccgcccggacccacagaccaccc

SEQ ID NO: 204 - DNA, 24 bp, Artificial Sequence
  Description of Artificial Sequence Synthetic oligonucleotide
cccggacccacagaccaccctcaa

SEQ ID NO: 205 - DNA, 24 bp, Artificial Sequence
  Description of Artificial Sequence Synthetic oligonucleotide
ccaggacggttgagggtggtctgt

SEQ ID NO: 206 - DNA, 21 bp, Artificial Sequence
  Description of Artificial Sequence Synthetic oligonucleotide
tccggggccaggacggttgag

SEQ ID NO: 207 - DNA, 20 bp, Artificial Sequence
  Description of Artificial Sequence Synthetic oligonucleotide
ccctgggcaggttggtttca

SEQ ID NO: 208 - DNA, 20 bp, Artificial Sequence
  Description of Artificial Sequence Synthetic oligonucleotide
aggttggtttcaaggttaca

SEQ ID NO: 209 - DNA, 20 bp, Artificial Sequence
  Description of Artificial Sequence Synthetic oligonucleotide
ttgtaaccttgaaaccaacc

SEQ ID NO: 210 - DNA, 20 bp, Artificial Sequence
  Description of Artificial Sequence Synthetic oligonucleotide
cctgggcaggttggtttcaa

SEQ ID NO: 211 - DNA, 23 bp, Artificial Sequence
  Description of Artificial Sequence Synthetic oligonucleotide
aacctgtcttgtaaccttgaaac

SEQ ID NO: 212 - DNA, 24 bp, Artificial Sequence
  Description of Artificial Sequence Synthetic oligonucleotide
tccttaaacctgtcttgtaacctt

SEQ ID NO: 213 - DNA, 24 bp, Artificial Sequence
  Description of Artificial Sequence Synthetic oligonucleotide
taaacctgtcttgtaaccttgaaa

SEQ ID NO: 214 - DNA, 24 bp, Artificial Sequence
  Description of Artificial Sequence Synthetic oligonucleotide
cctgtcttgtaaccttgaaaccaa

SEQ ID NO: 215 - DNA, 24 bp, Artificial Sequence
  Description of Artificial Sequence Synthetic oligonucleotide
ctgtcttgtaaccttgaaaccaac

SEQ ID NO: 216 - DNA, 21 bp, Artificial Sequence
  Description of Artificial Sequence Synthetic oligonucleotide
aggccctgggcaggttggttt

SEQ ID NO: 217 - DNA, 20 bp, Artificial Sequence
  Description of Artificial Sequence Synthetic oligonucleotide
agaacttcagggtgactcta

SEQ ID NO: 218 - DNA, 20 bp, Artificial Sequence
  Description of Artificial Sequence Synthetic oligonucleotide
gaacttcagggtgactctat

SEQ ID NO: 219 - DNA, 20 bp, Artificial Sequence
  Description of Artificial Sequence Synthetic oligonucleotide
aacttcagggtgactctatg

SEQ ID NO: 220 - DNA, 20 bp, Artificial Sequence
  Description of Artificial Sequence Synthetic oligonucleotide
agcgtcccatagagtcaccc

SEQ ID NO: 221 - DNA, 20 bp, Artificial Sequence
  Description of Artificial Sequence Synthetic oligonucleotide
ttcagggtgactctatggga

SEQ ID NO: 222 - DNA, 20 bp, Artificial Sequence
  Description of Artificial Sequence Synthetic oligonucleotide
aaacatcaagcgtcccatag

SEQ ID NO: 223 - DNA, 20 bp, Artificial Sequence
  Description of Artificial Sequence Synthetic oligonucleotide
gtcccatagagtcaccctga

SEQ ID NO: 224 - DNA, 21 bp, Artificial Sequence
  Description of Artificial Sequence Synthetic oligonucleotide
aagaaaacatcaagcgtccca

SEQ ID NO: 225 - DNA, 24 bp, Artificial Sequence
  Description of Artificial Sequence Synthetic oligonucleotide
agaaaacatcaagcgtcccataga

SEQ ID NO: 226 - DNA, 24 bp, Artificial Sequence
  Description of Artificial Sequence Synthetic oligonucleotide
gaaaacatcaagcgtcccatagag

SEQ ID NO: 227 - DNA, 21 bp, Artificial Sequence
  Description of Artificial Sequence Synthetic oligonucleotide
tcagggtgactctatgggacg

SEQ ID NO: 228 - DNA, 21 bp, Artificial Sequence
  Description of Artificial Sequence Synthetic oligonucleotide
aagcgtcccatagagtcaccc

SEQ ID NO: 229 - DNA, 20 bp, Artificial Sequence
  Description of Artificial Sequence Synthetic oligonucleotide
ttctttagaatggtacaaag

SEQ ID NO: 230 - DNA, 20 bp, Artificial Sequence
  Description of Artificial Sequence Synthetic oligonucleotide
gcctctttgtaccattctaa

SEQ ID NO: 231 - DNA, 20 bp, Artificial Sequence
  Description of Artificial Sequence Synthetic oligonucleotide
tattctttagaatggtacaa

SEQ ID NO: 232 - DNA, 20 bp, Artificial Sequence
  Description of Artificial Sequence Synthetic oligonucleotide
tagaatggtacaaagaggca

SEQ ID NO: 233 - DNA, 20 bp, Artificial Sequence
  Description of Artificial Sequence Synthetic oligonucleotide
tctttagaatggtacaaaga

SEQ ID NO: 234 - DNA, 20 bp, Artificial Sequence
  Description of Artificial Sequence Synthetic oligonucleotide
tacaatgtatcatgcctctt

SEQ ID NO: 235 - DNA, 21 bp, Artificial Sequence
  Description of Artificial Sequence Synthetic oligonucleotide
atgcctctttgtaccattcta

SEQ ID NO: 236 - DNA, 22 bp, Artificial Sequence
  Description of Artificial Sequence Synthetic oligonucleotide
aatgatacaatgtatcatgcct

SEQ ID NO: 237 - DNA, 22 bp, Artificial Sequence
  Description of Artificial Sequence Synthetic oligonucleotide
tctttagaatggtacaaagagg

SEQ ID NO: 238 - DNA, 23 bp, Artificial Sequence
  Description of Artificial Sequence Synthetic oligonucleotide
ttctttagaatggtacaaagagg

SEQ ID NO: 239 - DNA, 23 bp, Artificial Sequence
  Description of Artificial Sequence Synthetic oligonucleotide
tttagaatggtacaaagaggcat

SEQ ID NO: 240 - DNA, 21 bp, Artificial Sequence
  Description of Artificial Sequence Synthetic oligonucleotide
atcactgttattctttagaat

SEQ ID NO: 241 - DNA, 20 bp, Artificial Sequence
  Description of Artificial Sequence Synthetic oligonucleotide
atgcctctttgtaccattct

SEQ ID NO: 242 - DNA, 23 bp, Artificial Sequence
  Description of Artificial Sequence Synthetic oligonucleotide
atgcctctttgtaccattctaaa

SEQ ID NO: 243 - DNA, 23 bp, Artificial Sequence
  Description of Artificial Sequence Synthetic oligonucleotide
actgttattctttagaatggtac

SEQ ID NO: 244 - DNA, 24 bp, Artificial Sequence
  Description of Artificial Sequence Synthetic oligonucleotide
atgatacaatgtatcatgcctctt

SEQ ID NO: 245 - DNA, 21 bp, Artificial Sequence
  Description of Artificial Sequence Synthetic oligonucleotide
ctttagaatggtacaaagagg

SEQ ID NO: 246 - DNA, 20 bp, Artificial Sequence
  Description of Artificial Sequence Synthetic oligonucleotide
tattgctattaccttaaccc

SEQ ID NO: 247 - DNA, 20 bp, Artificial Sequence
  Description of Artificial Sequence Synthetic oligonucleotide
aatttctgggttaaggtaat

SEQ ID NO: 248 - DNA, 20 bp, Artificial Sequence
  Description of Artificial Sequence Synthetic oligonucleotide
agtgataatttctgggttaa

SEQ ID NO: 249 - DNA, 23 bp, Artificial Sequence
  Description of Artificial Sequence Synthetic oligonucleotide
tgggttaaggtaatagcaatatc

SEQ ID NO: 250 - DNA, 23 bp, Artificial Sequence
  Description of Artificial Sequence Synthetic oligonucleotide
tatgcagagatattgctattacc

SEQ ID NO: 251 - DNA, 23 bp, Artificial Sequence
  Description of Artificial Sequence Synthetic oligonucleotide
ctgggttaaggtaatagcaatat

SEQ ID NO: 252 - DNA, 23 bp, Artificial Sequence
  Description of Artificial Sequence Synthetic oligonucleotide
tgggttaaggtaatagcaatatc

SEQ ID NO: 253 - DNA, 23 bp, Artificial Sequence
  Description of Artificial Sequence Synthetic oligonucleotide
atatgcagagatattgctattac

SEQ ID NO: 254 - DNA, 23 bp, Artificial Sequence
  Description of Artificial Sequence Synthetic oligonucleotide
tatgcagagatattgctattacc

SEQ ID NO: 255 - DNA, 20 bp, Artificial Sequence
  Description of Artificial Sequence Synthetic oligonucleotide
gatattgctattaccttaac

SEQ ID NO: 256 - DNA, 23 bp, Artificial Sequence
  Description of Artificial Sequence Synthetic oligonucleotide
tgcagagatattgctattacctt

SEQ ID NO: 257 - DNA, 23 bp, Artificial Sequence
  Description of Artificial Sequence Synthetic oligonucleotide
cagagatattgctattaccttaa

SEQ ID NO: 258 - DNA, 24 bp, Artificial Sequence
  Description of Artificial Sequence Synthetic oligonucleotide
atatttatatgcagagatattgct

SEQ ID NO: 259 - DNA, 24 bp, Artificial Sequence
  Description of Artificial Sequence Synthetic oligonucleotide
atatgcagagatattgctattacc

SEQ ID NO: 260 - DNA, 24 bp, Artificial Sequence
  Description of Artificial Sequence Synthetic oligonucleotide
tatgcagagatattgctattacct

SEQ ID NO: 261 - DNA, 21 bp, Artificial Sequence
  Description of Artificial Sequence Synthetic oligonucleotide
taacagtgataatttctgggt

SEQ ID NO: 262 - DNA, 21 bp, Artificial Sequence
  Description of Artificial Sequence Synthetic oligonucleotide
cagtgataatttctgggttaa

SEQ ID NO: 263 - DNA, 21 bp, Artificial Sequence
  Description of Artificial Sequence Synthetic oligonucleotide
taatttctgggttaaggtaat

SEQ ID NO: 264 - DNA, 21 bp, Artificial Sequence
  Description of Artificial Sequence Synthetic oligonucleotide
atattgctattaccttaaccc

SEQ ID NO: 265 - DNA, 20 bp, Artificial Sequence
  Description of Artificial Sequence Synthetic oligonucleotide
ctgcatataaattgtaactg

SEQ ID NO: 266 - DNA, 20 bp, Artificial Sequence
  Description of Artificial Sequence Synthetic oligonucleotide
taaattgtaactgaggtaag

SEQ ID NO: 267 - DNA, 20 bp, Artificial Sequence
  Description of Artificial Sequence Synthetic oligonucleotide
tataaattgtaactgaggta

SEQ ID NO: 268 - DNA, 20 bp, Artificial Sequence
  Description of Artificial Sequence Synthetic oligonucleotide
tgcatataaattgtaactga

SEQ ID NO: 269 - DNA, 20 bp, Artificial Sequence
  Description of Artificial Sequence Synthetic oligonucleotide
aaattgtaactgaggtaaga

SEQ ID NO: 270 - DNA, 20 bp, Artificial Sequence
  Description of Artificial Sequence Synthetic oligonucleotide
aatatgaaacctcttacctc

SEQ ID NO: 271 - DNA, 23 bp, Artificial Sequence
  Description of Artificial Sequence Synthetic oligonucleotide
tgcatataaattgtaactgaggt

SEQ ID NO: 272 - DNA, 23 bp, Artificial Sequence
  Description of Artificial Sequence Synthetic oligonucleotide
ctgcatataaattgtaactgagg

SEQ ID NO: 273 - DNA, 23 bp, Artificial Sequence
  Description of Artificial Sequence Synthetic oligonucleotide
tgcatataaattgtaactgaggt

SEQ ID NO: 274 - DNA, 23 bp, Artificial Sequence
  Description of Artificial Sequence Synthetic oligonucleotide
gcaatatgaaacctcttacctca

SEQ ID NO: 275 - DNA, 23 bp, Artificial Sequence
  Description of Artificial Sequence Synthetic oligonucleotide
aattgtaactgaggtaagaggtt

SEQ ID NO: 276 - DNA, 23 bp, Artificial Sequence
  Description of Artificial Sequence Synthetic oligonucleotide
aaacctcttacctcagttacaat

SEQ ID NO: 277 - DNA, 24 bp, Artificial Sequence
  Description of Artificial Sequence Synthetic oligonucleotide
gctgctattagcaatatgaaacct

SEQ ID NO: 278 - DNA, 21 bp, Artificial Sequence
  Description of Artificial Sequence Synthetic oligonucleotide
tttctgcatataaattgtaac

SEQ ID NO: 279 - DNA, 21 bp, Artificial Sequence
  Description of Artificial Sequence Synthetic oligonucleotide
atataaattgtaactgaggta

SEQ ID NO: 280 - DNA, 21 bp, Artificial Sequence
  Description of Artificial Sequence Synthetic oligonucleotide
tagcaatatgaaacctcttac

SEQ ID NO: 281 - DNA, 21 bp, Artificial Sequence
  Description of Artificial Sequence Synthetic oligonucleotide
tatgaaacctcttacctcagt

SEQ ID NO: 282 - DNA, 20 bp, Artificial Sequence
  Description of Artificial Sequence Synthetic oligonucleotide
tgtaagaggtttcatattgc

SEQ ID NO: 283 - DNA, 20 bp, Artificial Sequence
  Description of Artificial Sequence Synthetic oligonucleotide
tgtagctgctatcagcaata

SEQ ID NO: 284 - DNA, 20 bp, Artificial Sequence
  Description of Artificial Sequence Synthetic oligonucleotide
agaggtttcatattgctgat

SEQ ID NO: 285 - DNA, 20 bp, Artificial Sequence
  Description of Artificial Sequence Synthetic oligonucleotide
ggtttcatattgctgatagc

SEQ ID NO: 286 - DNA, 20 bp, Artificial Sequence
  Description of Artificial Sequence Synthetic oligonucleotide
gctggattgtagctgctatc

SEQ ID NO: 287 - DNA, 23 bp, Artificial Sequence
  Description of Artificial Sequence Synthetic oligonucleotide
tagctgctatcagcaatatgaaa

SEQ ID NO: 288 - DNA, 24 bp, Artificial Sequence
  Description of Artificial Sequence Synthetic oligonucleotide
gtttcatattgctgatagcagcta

SEQ ID NO: 289 - DNA, 24 bp, Artificial Sequence
  Description of Artificial Sequence Synthetic oligonucleotide
gattgtagctgctatcagcaatat

SEQ ID NO: 290 - DNA, 21 bp, Artificial Sequence
  Description of Artificial Sequence Synthetic oligonucleotide
tgatgtaagaggtttcatatt

SEQ ID NO: 291 - DNA, 21 bp, Artificial Sequence
  Description of Artificial Sequence Synthetic oligonucleotide
tttcatattgctgatagcagc

SEQ ID NO: 292 - DNA, 21 bp, Artificial Sequence
  Description of Artificial Sequence Synthetic oligonucleotide
agctggattgtagctgctatc

SEQ ID NO: 293 - DNA, 20 bp, Artificial Sequence
  Description of Artificial Sequence Synthetic oligonucleotide
gctaatagcagctacaatcc

SEQ ID NO: 294 - DNA, 20 bp, Artificial Sequence
  Description of Artificial Sequence Synthetic oligonucleotide
aataaaagcagaatggtacc

SEQ ID NO: 295 - DNA, 20 bp, Artificial Sequence
  Description of Artificial Sequence Synthetic oligonucleotide
ataaaagcagaatggtacct

SEQ ID NO: 296 - DNA, 20 bp, Artificial Sequence
  Description of Artificial Sequence Synthetic oligonucleotide
ctacaatccaggtaccattc

SEQ ID NO: 297 - DNA, 20 bp, Artificial Sequence
  Description of Artificial Sequence Synthetic oligonucleotide
cagaatggtacctggattgt

SEQ ID NO: 298 - DNA, 20 bp, Artificial Sequence
  Description of Artificial Sequence Synthetic oligonucleotide
ctaatagcagctacaatcca

SEQ ID NO: 299 - DNA, 20 bp, Artificial Sequence
  Description of Artificial Sequence Synthetic oligonucleotide
aagcagaatggtacctggat

SEQ ID NO: 300 - DNA, 24 bp, Artificial Sequence
  Description of Artificial Sequence Synthetic oligonucleotide
ccataaaataaaagcagaatggta

SEQ ID NO: 301 - DNA, 21 bp, Artificial Sequence
  Description of Artificial Sequence Synthetic oligonucleotide
aaaataaaagcagaatggtac

SEQ ID NO: 302 - DNA, 22 bp, Artificial Sequence
  Description of Artificial Sequence Synthetic oligonucleotide
tattgctaatagcagctacaat

SEQ ID NO: 303 - DNA, 23 bp, Artificial Sequence
  Description of Artificial Sequence Synthetic oligonucleotide
ctaatagcagctacaatccaggt

SEQ ID NO: 304 - DNA, 24 bp, Artificial Sequence
  Description of Artificial Sequence Synthetic oligonucleotide
attgctaatagcagctacaatcca

SEQ ID NO: 305 - DNA, 24 bp, Artificial Sequence
  Description of Artificial Sequence Synthetic oligonucleotide
ccaaccataaaataaaagcagaat

SEQ ID NO: 306 - DNA, 21 bp, Artificial Sequence
  Description of Artificial Sequence Synthetic oligonucleotide
attgctaatagcagctacaat

SEQ ID NO: 307 - DNA, 20 bp, Artificial Sequence
  Description of Artificial Sequence Synthetic oligonucleotide
accattctgcttttgtttta

SEQ ID NO: 308 - DNA, 20 bp, Artificial Sequence
  Description of Artificial Sequence Synthetic oligonucleotide
ttctgcttttgttttatggt

SEQ ID NO: 309 - DNA, 20 bp, Artificial Sequence
  Description of Artificial Sequence Synthetic oligonucleotide
tctgcttttgttttatggtt

SEQ ID NO: 310 - DNA, 20 bp, Artificial Sequence
  Description of Artificial Sequence Synthetic oligonucleotide
accataaaacaaaagcagaa

SEQ ID NO: 311 - DNA, 20 bp, Artificial Sequence
  Description of Artificial Sequence Synthetic oligonucleotide
ctgcttttgttttatggttg

SEQ ID NO: 312 - DNA, 20 bp, Artificial Sequence
  Description of Artificial Sequence Synthetic oligonucleotide
cccaaccataaaacaaaagc

SEQ ID NO: 313 - DNA, 20 bp, Artificial Sequence
  Description of Artificial Sequence Synthetic oligonucleotide
tatcccaaccataaaacaaa

SEQ ID NO: 314 - DNA, 20 bp, Artificial Sequence
  Description of Artificial Sequence Synthetic oligonucleotide
tccagctaccattctgcttt

SEQ ID NO: 315 - DNA, 20 bp, Artificial Sequence
  Description of Artificial Sequence Synthetic oligonucleotide
ccattctgcttttgttttat

SEQ ID NO: 316 - DNA, 20 bp, Artificial Sequence
  Description of Artificial Sequence Synthetic oligonucleotide
ccataaaacaaaagcagaat

SEQ ID NO: 317 - DNA, 21 bp, Artificial Sequence
  Description of Artificial Sequence Synthetic oligonucleotide
attctgcttttgttttatggt

SEQ ID NO: 318 - DNA, 21 bp, Artificial Sequence
  Description of Artificial Sequence Synthetic oligonucleotide
atcccaaccataaaacaaaag

SEQ ID NO: 319 - DNA, 23 bp, Artificial Sequence
  Description of Artificial Sequence Synthetic oligonucleotide
tcccaaccataaaacaaaagcag

SEQ ID NO: 320 - DNA, 21 bp, Artificial Sequence
  Description of Artificial Sequence Synthetic oligonucleotide
atccagccttatcccaaccat

SEQ ID NO: 321 - DNA, 20 bp, Artificial Sequence
  Description of Artificial Sequence Synthetic oligonucleotide
atcccaaccataaaacaaaa

SEQ ID NO: 322 - DNA, 23 bp, Artificial Sequence
  Description of Artificial Sequence Synthetic oligonucleotide
gctaccattctgcttttgtttta

SEQ ID NO: 323 - DNA, 23 bp, Artificial Sequence
  Description of Artificial Sequence Synthetic oligonucleotide
gccttatcccaaccataaaacaa

SEQ ID NO: 324 - DNA, 23 bp, Artificial Sequence
  Description of Artificial Sequence Synthetic oligonucleotide
aaccataaaacaaaagcagaatg

SEQ ID NO: 325 - DNA, 23 bp, Artificial Sequence
  Description of Artificial Sequence Synthetic oligonucleotide
ccaaccataaaacaaaagcagaa

SEQ ID NO: 326 - DNA, 21 bp, Artificial Sequence
  Description of Artificial Sequence Synthetic oligonucleotide
gctaccattctgcttttgttt

SEQ ID NO: 327 - DNA, 21 bp, Artificial Sequence
  Description of Artificial Sequence Synthetic oligonucleotide
ccaaccataaaacaaaagcag

SEQ ID NO: 328 - DNA, 20 bp, Artificial Sequence
  Description of Artificial Sequence Synthetic oligonucleotide
ttattttatggttgggataa

SEQ ID NO: 329 - DNA, 20 bp, Artificial Sequence
  Description of Artificial Sequence Synthetic oligonucleotide
ttttatggttgggataaggg

SEQ ID NO: 330 - DNA, 20 bp, Artificial Sequence
  Description of Artificial Sequence Synthetic oligonucleotide
tttatggttgggataagggt

SEQ ID NO: 331 - DNA, 20 bp, Artificial Sequence
  Description of Artificial Sequence Synthetic oligonucleotide
gggataagggtggattattc

SEQ ID NO: 332 - DNA, 20 bp, Artificial Sequence
  Description of Artificial Sequence Synthetic oligonucleotide
tattttatggttgggataag

SEQ ID NO: 333 - DNA, 24 bp, Artificial Sequence
  Description of Artificial Sequence Synthetic oligonucleotide
cttttattttatggttgggataag

SEQ ID NO: 334 - DNA, 21 bp, Artificial Sequence
  Description of Artificial Sequence Synthetic oligonucleotide
cttttattttatggttgggat

SEQ ID NO: 335 - DNA, 21 bp, Artificial Sequence
  Description of Artificial Sequence Synthetic oligonucleotide
tattttatggttgggataagg

SEQ ID NO: 336 - DNA, 21 bp, Artificial Sequence
  Description of Artificial Sequence Synthetic oligonucleotide
ttgggataagggtggattatt

SEQ ID NO: 337 - DNA, 23 bp, Artificial Sequence
  Description of Artificial Sequence Synthetic oligonucleotide
ttttatggttgggataagggtgg

SEQ ID NO: 338 - DNA, 23 bp, Artificial Sequence
  Description of Artificial Sequence Synthetic oligonucleotide
tggttgggataagggtggattat

SEQ ID NO: 339 - DNA, 23 bp, Artificial Sequence
  Description of Artificial Sequence Synthetic oligonucleotide
tattttatggttgggataagggt

SEQ ID NO: 340 - DNA, 23 bp, Artificial Sequence
  Description of Artificial Sequence Synthetic oligonucleotide
attttatggttgggataagggtg

SEQ ID NO: 341 - DNA, 23 bp, Artificial Sequence
  Description of Artificial Sequence Synthetic oligonucleotide
ttttatggttgggataagggtgg

SEQ ID NO: 342 - DNA, 23 bp, Artificial Sequence
  Description of Artificial Sequence Synthetic oligonucleotide
tatggttgggataagggtggatt

SEQ ID NO: 343 - DNA, 23 bp, Artificial Sequence
  Description of Artificial Sequence Synthetic oligonucleotide
atggttgggataagggtggatta

SEQ ID NO: 344 - DNA, 23 bp, Artificial Sequence
  Description of Artificial Sequence Synthetic oligonucleotide
tggttgggataagggtggattat

SEQ ID NO: 345 - DNA, 23 bp, Artificial Sequence
  Description of Artificial Sequence Synthetic oligonucleotide
gactcagaataatccacccttat

SEQ ID NO: 346 - DNA, 20 bp, Artificial Sequence
  Description of Artificial Sequence Synthetic oligonucleotide
ttattttatggttgggataa

SEQ ID NO: 347 - DNA, 23 bp, Artificial Sequence
  Description of Artificial Sequence Synthetic oligonucleotide
gttgggataagggtggattattc

SEQ ID NO: 348 - DNA, 24 bp, Artificial Sequence
  Description of Artificial Sequence Synthetic oligonucleotide
gttgggataagggtggattattct

SEQ ID NO: 349 - DNA, 24 bp, Artificial Sequence
  Description of Artificial Sequence Synthetic oligonucleotide
gggcctagcttggactcagaataa

SEQ ID NO: 350 - DNA, 24 bp, Artificial Sequence
  Description of Artificial Sequence Synthetic oligonucleotide
ggcctagcttggactcagaataat

SEQ ID NO: 351 - DNA, 24 bp, Artificial Sequence
  Description of Artificial Sequence Synthetic oligonucleotide
gcttggactcagaataatccaccc

SEQ ID NO: 352 - DNA, 24 bp, Artificial Sequence
  Description of Artificial Sequence Synthetic oligonucleotide
cttggactcagaataatccaccct

SEQ ID NO: 353 - DNA, 24 bp, Artificial Sequence
  Description of Artificial Sequence Synthetic oligonucleotide
gactcagaataatccacccttatc

SEQ ID NO: 354 - DNA, 20 bp, Artificial Sequence
  Description of Artificial Sequence Synthetic oligonucleotide
ttttatggttgggataaggt

SEQ ID NO: 355 - DNA, 20 bp, Artificial Sequence
  Description of Artificial Sequence Synthetic oligonucleotide
tttatggttgggataaggtt

SEQ ID NO: 356 - DNA, 20 bp, Artificial Sequence
  Description of Artificial Sequence Synthetic oligonucleotide
gggataaggttggattattc

SEQ ID NO: 357 - DNA, 20 bp, Artificial Sequence
  Description of Artificial Sequence Synthetic oligonucleotide
ttattttatggttgggataa

SEQ ID NO: 358 - DNA, 24 bp, Artificial Sequence
  Description of Artificial Sequence Synthetic oligonucleotide
cttttattttatggttgggataag

SEQ ID NO: 359 - DNA, 21 bp, Artificial Sequence
  Description of Artificial Sequence Synthetic oligonucleotide
tattttatggttgggataagg

SEQ ID NO: 360 - DNA, 21 bp, Artificial Sequence
  Description of Artificial Sequence Synthetic oligonucleotide
ttgggataaggttggattatt

SEQ ID NO: 361 - DNA, 23 bp, Artificial Sequence
  Description of Artificial Sequence Synthetic oligonucleotide
ttttatggttgggataaggttgg

SEQ ID NO: 362 - DNA, 23 bp, Artificial Sequence
  Description of Artificial Sequence Synthetic oligonucleotide
tggttgggataaggttggattat

SEQ ID NO: 363 - DNA, 23 bp, Artificial Sequence
  Description of Artificial Sequence Synthetic oligonucleotide
tattttatggttgggataaggtt

SEQ ID NO: 364 - DNA, 23 bp, Artificial Sequence
  Description of Artificial Sequence Synthetic oligonucleotide
attttatggttgggataaggttg

SEQ ID NO: 365 - DNA, 23 bp, Artificial Sequence
  Description of Artificial Sequence Synthetic oligonucleotide
ttttatggttgggataaggttgg

SEQ ID NO: 366 - DNA, 23 bp, Artificial Sequence
  Description of Artificial Sequence Synthetic oligonucleotide
tatggttgggataaggttggatt

SEQ ID NO: 367 - DNA, 23 bp, Artificial Sequence
  Description of Artificial Sequence Synthetic oligonucleotide
atggttgggataaggttggatta

SEQ ID NO: 368 - DNA, 23 bp, Artificial Sequence
  Description of Artificial Sequence Synthetic oligonucleotide
tggttgggataaggttggattat

SEQ ID NO: 369 - DNA, 23 bp, Artificial Sequence
  Description of Artificial Sequence Synthetic oligonucleotide
gactcagaataatccaaccttat

SEQ ID NO: 370 - DNA, 23 bp, Artificial Sequence
  Description of Artificial Sequence Synthetic oligonucleotide
gttgggataaggttggattattc

SEQ ID NO: 371 - DNA, 24 bp, Artificial Sequence
  Description of Artificial Sequence Synthetic oligonucleotide
gttgggataaggttggattattct

SEQ ID NO: 372 - DNA, 24 bp, Artificial Sequence
  Description of Artificial Sequence Synthetic oligonucleotide
ggcctagcttggactcagaataat

SEQ ID NO: 373 - DNA, 24 bp, Artificial Sequence
  Description of Artificial Sequence Synthetic oligonucleotide
gcttggactcagaataatccaacc

SEQ ID NO: 374 - DNA, 24 bp, Artificial Sequence
  Description of Artificial Sequence Synthetic oligonucleotide
cttggactcagaataatccaacct

SEQ ID NO: 375 - DNA, 24 bp, Artificial Sequence
  Description of Artificial Sequence Synthetic oligonucleotide
gactcagaataatccaaccttatc

SEQ ID NO: 376 - DNA, 21 bp, Artificial Sequence
  Description of Artificial Sequence Synthetic oligonucleotide
gcttttattttatggttggga

SEQ ID NO: 377 - DNA, 20 bp, Artificial Sequence
  Description of Artificial Sequence Synthetic oligonucleotide
agctgtgggaggaagctaag

SEQ ID NO: 378 - DNA, 20 bp, Artificial Sequence
  Description of Artificial Sequence Synthetic oligonucleotide
ggagctgtgggaggaagcta

SEQ ID NO: 379 - DNA, 20 bp, Artificial Sequence
  Description of Artificial Sequence Synthetic oligonucleotide
tgggaggaagctaagaggta

SEQ ID NO: 380 - DNA, 20 bp, Artificial Sequence
  Description of Artificial Sequence Synthetic oligonucleotide
taatcatgttcatacctctt

SEQ ID NO: 381 - DNA, 20 bp, Artificial Sequence
  Description of Artificial Sequence Synthetic oligonucleotide
acctcttagcttcctcccac

SEQ ID NO: 382 - DNA, 20 bp, Artificial Sequence
  Description of Artificial Sequence Synthetic oligonucleotide
gcccaggagctgtgggagga

SEQ ID NO: 383 - DNA, 20 bp, Artificial Sequence
  Description of Artificial Sequence Synthetic oligonucleotide
gctgtgggaggaagctaaga

SEQ ID NO: 384 - DNA, 23 bp, Artificial Sequence
  Description of Artificial Sequence Synthetic oligonucleotide
ctaatcatgttcatacctcttag

SEQ ID NO: 385 - DNA, 23 bp, Artificial Sequence
  Description of Artificial Sequence Synthetic oligonucleotide
ctaatcatgttcatacctcttag

SEQ ID NO: 386 - DNA, 23 bp, Artificial Sequence
  Description of Artificial Sequence Synthetic oligonucleotide
atacctcttagcttcctcccaca

SEQ ID NO: 387 - DNA, 23 bp, Artificial Sequence
  Description of Artificial Sequence Synthetic oligonucleotide
cccaggagctgtgggaggaagct

SEQ ID NO: 388 - DNA, 24 bp, Artificial Sequence
  Description of Artificial Sequence Synthetic oligonucleotide
tgctaatcatgttcatacctctta

SEQ ID NO: 389 - DNA, 24 bp, Artificial Sequence
  Description of Artificial Sequence Synthetic oligonucleotide
taatcatgttcatacctcttagct

SEQ ID NO: 390 - DNA, 24 bp, Artificial Sequence
  Description of Artificial Sequence Synthetic oligonucleotide
aatcatgttcatacctcttagctt

SEQ ID NO: 391 - DNA, 24 bp, Artificial Sequence
  Description of Artificial Sequence Synthetic oligonucleotide
tcatacctcttagcttcctcccac

SEQ ID NO: 392 - DNA, 21 bp, Artificial Sequence
  Description of Artificial Sequence Synthetic oligonucleotide
aggagctgtgggaggaagcta

SEQ ID NO: 393 - DNA, 20 bp, Artificial Sequence
  Description of Artificial Sequence Synthetic oligonucleotide
tatcttccgcccacagctcc

SEQ ID NO: 394 - DNA, 20 bp, Artificial Sequence
  Description of Artificial Sequence Synthetic oligonucleotide
cgttgcccaggagctgtggg

SEQ ID NO: 395 - DNA, 20 bp, Artificial Sequence
  Description of Artificial Sequence Synthetic oligonucleotide
agctgtgggcggaagataag

SEQ ID NO: 396 - DNA, 20 bp, Artificial Sequence
  Description of Artificial Sequence Synthetic oligonucleotide
cacgttgcccaggagctgtg

SEQ ID NO: 397 - DNA, 20 bp, Artificial Sequence
  Description of Artificial Sequence Synthetic oligonucleotide
gttgcccaggagctgtgggc

SEQ ID NO: 398 - DNA, 20 bp, Artificial Sequence
  Description of Artificial Sequence Synthetic oligonucleotide
gcccaggagctgtgggcgga

SEQ ID NO: 399 - DNA, 20 bp, Artificial Sequence
  Description of Artificial Sequence Synthetic oligonucleotide
ggagctgtgggcggaagata

SEQ ID NO: 400 - DNA, 20 bp, Artificial Sequence
  Description of Artificial Sequence Synthetic oligonucleotide
tgttcatacctcttatcttc

SEQ ID NO: 401 - DNA, 20 bp, Artificial Sequence
  Description of Artificial Sequence Synthetic oligonucleotide
acctcttatcttccgcccac

SEQ ID NO: 402 - DNA, 20 bp, Artificial Sequence
  Description of Artificial Sequence Synthetic oligonucleotide
cacgttgcccaggagctgtg

SEQ ID NO: 403 - DNA, 20 bp, Artificial Sequence
  Description of Artificial Sequence Synthetic oligonucleotide
gctgtgggcggaagataaga

SEQ ID NO: 404 - DNA, 23 bp, Artificial Sequence
  Description of Artificial Sequence Synthetic oligonucleotide
atacctcttatcttccgcccaca

SEQ ID NO: 405 - DNA, 23 bp, Artificial Sequence
  Description of Artificial Sequence Synthetic oligonucleotide
cccaggagctgtgggcggaagat

SEQ ID NO: 406 - DNA, 20 bp, Artificial Sequence
  Description of Artificial Sequence Synthetic oligonucleotide
ttgcccaggagctgtgggcg

SEQ ID NO: 407 - DNA, 20 bp, Artificial Sequence
  Description of Artificial Sequence Synthetic oligonucleotide
gcacgttgcccaggagctgt

SEQ ID NO: 408 - DNA, 23 bp, Artificial Sequence
  Description of Artificial Sequence Synthetic oligonucleotide
tacctcttatcttccgcccacag

SEQ ID NO: 409 - DNA, 24 bp, Artificial Sequence
  Description of Artificial Sequence Synthetic oligonucleotide
taatcatgttcatacctcttatct

SEQ ID NO: 410 - DNA, 24 bp, Artificial Sequence
  Description of Artificial Sequence Synthetic oligonucleotide
aatcatgttcatacctcttatctt

SEQ ID NO: 411 - DNA, 24 bp, Artificial Sequence
  Description of Artificial Sequence Synthetic oligonucleotide
tcatacctcttatcttccgcccac

SEQ ID NO: 412 - DNA, 21 bp, Artificial Sequence
  Description of Artificial Sequence Synthetic oligonucleotide
gttgcccaggagctgtgggcg

SEQ ID NO: 413 - DNA, 21 bp, Artificial Sequence
  Description of Artificial Sequence Synthetic oligonucleotide
aggagctgtgggcggaagata

SEQ ID NO: 414 - DNA, 20 bp, Artificial Sequence
  Description of Artificial Sequence Synthetic oligonucleotide
tatcttcctaccacagctcc

SEQ ID NO: 415 - DNA, 20 bp, Artificial Sequence
  Description of Artificial Sequence Synthetic oligonucleotide
atcttcctaccacagctcct

SEQ ID NO: 416 - DNA, 20 bp, Artificial Sequence
  Description of Artificial Sequence Synthetic oligonucleotide
cgttgcccaggagctgtggt

SEQ ID NO: 417 - DNA, 20 bp, Artificial Sequence
  Description of Artificial Sequence Synthetic oligonucleotide
agctgtggtaggaagataag

SEQ ID NO: 418 - DNA, 20 bp, Artificial Sequence
  Description of Artificial Sequence Synthetic oligonucleotide
gttgcccaggagctgtggta

SEQ ID NO: 419 - DNA, 20 bp, Artificial Sequence
  Description of Artificial Sequence Synthetic oligonucleotide
gcccaggagctgtggtagga

SEQ ID NO: 420 - DNA, 20 bp, Artificial Sequence
  Description of Artificial Sequence Synthetic oligonucleotide
ggagctgtggtaggaagata

SEQ ID NO: 421 - DNA, 20 bp, Artificial Sequence
  Description of Artificial Sequence Synthetic oligonucleotide
acctcttatcttcctaccac

SEQ ID NO: 422 - DNA, 20 bp, Artificial Sequence
  Description of Artificial Sequence Synthetic oligonucleotide
gcacgttgcccaggagctgt

SEQ ID NO: 423 - DNA, 23 bp, Artificial Sequence
  Description of Artificial Sequence Synthetic oligonucleotide
atacctcttatcttcctaccaca

SEQ ID NO: 424 - DNA, 23 bp, Artificial Sequence
  Description of Artificial Sequence Synthetic oligonucleotide
cccaggagctgtggtaggaagat

SEQ ID NO: 425 - DNA, 20 bp, Artificial Sequence
  Description of Artificial Sequence Synthetic oligonucleotide
ttgcccaggagctgtggtag

SEQ ID NO: 426 - DNA, 23 bp, Artificial Sequence
  Description of Artificial Sequence Synthetic oligonucleotide
tacctcttatcttcctaccacag

SEQ ID NO: 427 - DNA, 24 bp, Artificial Sequence
  Description of Artificial Sequence Synthetic oligonucleotide
aatcatgttcatacctcttatctt

SEQ ID NO: 428 - DNA, 24 bp, Artificial Sequence
  Description of Artificial Sequence Synthetic oligonucleotide
tcatacctcttatcttcctaccac

SEQ ID NO: 429 - DNA, 21 bp, Artificial Sequence
  Description of Artificial Sequence Synthetic oligonucleotide
accagcacgttgcccaggagc

SEQ ID NO: 430 - DNA, 21 bp, Artificial Sequence
  Description of Artificial Sequence Synthetic oligonucleotide
gttgcccaggagctgtggtag

SEQ ID NO: 431 - DNA, 21 bp, Artificial Sequence
  Description of Artificial Sequence Synthetic oligonucleotide
aggagctgtggtaggaagata

SEQ ID NO: 432 - DNA, 20 bp, Artificial Sequence
  Description of Artificial Sequence Synthetic oligonucleotide
tatcttcctgccacagctcc

SEQ ID NO: 433 - DNA, 20 bp, Artificial Sequence
  Description of Artificial Sequence Synthetic oligonucleotide
atcttcctgccacagctcct

SEQ ID NO: 434 - DNA, 20 bp, Artificial Sequence
  Description of Artificial Sequence Synthetic oligonucleotide
cgttgcccaggagctgtggc

SEQ ID NO: 435 - DNA, 20 bp, Artificial Sequence
  Description of Artificial Sequence Synthetic oligonucleotide
agctgtggcaggaagataag

SEQ ID NO: 436 - DNA, 20 bp, Artificial Sequence
  Description of Artificial Sequence Synthetic oligonucleotide
gttgcccaggagctgtggca

SEQ ID NO: 437 - DNA, 20 bp, Artificial Sequence
  Description of Artificial Sequence Synthetic oligonucleotide
gcccaggagctgtggcagga

SEQ ID NO: 438 - DNA, 20 bp, Artificial Sequence
  Description of Artificial Sequence Synthetic oligonucleotide
ggagctgtggcaggaagata

SEQ ID NO: 439 - DNA, 20 bp, Artificial Sequence
  Description of Artificial Sequence Synthetic oligonucleotide
gttcatacctcttatcttcc

SEQ ID NO: 440 - DNA, 20 bp, Artificial Sequence
  Description of Artificial Sequence Synthetic oligonucleotide
acctcttatcttcctgccac

SEQ ID NO: 441 - DNA, 20 bp, Artificial Sequence
  Description of Artificial Sequence Synthetic oligonucleotide
gcacgttgcccaggagctgt

SEQ ID NO: 442 - DNA, 23 bp, Artificial Sequence
  Description of Artificial Sequence Synthetic oligonucleotide
atacctcttatcttcctgccaca

SEQ ID NO: 443 - DNA, 23 bp, Artificial Sequence
  Description of Artificial Sequence Synthetic oligonucleotide
cccaggagctgtggcaggaagat

SEQ ID NO: 444 - DNA, 20 bp, Artificial Sequence
  Description of Artificial Sequence Synthetic oligonucleotide
ttgcccaggagctgtggcag

SEQ ID NO: 445 - DNA, 23 bp, Artificial Sequence
  Description of Artificial Sequence Synthetic oligonucleotide
tacctcttatcttcctgccacag

SEQ ID NO: 446 - DNA, 24 bp, Artificial Sequence
  Description of Artificial Sequence Synthetic oligonucleotide
aatcatgttcatacctcttatctt

SEQ ID NO: 447 - DNA, 24 bp, Artificial Sequence
  Description of Artificial Sequence Synthetic oligonucleotide
tcatacctcttatcttcctgccac

SEQ ID NO: 448 - DNA, 21 bp, Artificial Sequence
  Description of Artificial Sequence Synthetic oligonucleotide
gttgcccaggagctgtggcag

SEQ ID NO: 449 - DNA, 21 bp, Artificial Sequence
  Description of Artificial Sequence Synthetic oligonucleotide
aggagctgtggcaggaagata

SEQ ID NO: 450 - DNA, 20 bp, Artificial Sequence
  Description of Artificial Sequence Synthetic oligonucleotide
tttaaatacacacattttta

SEQ ID NO: 451 - DNA, 20 bp, Artificial Sequence
  Description of Artificial Sequence Synthetic oligonucleotide
tcctggtttgcttaaaaatg

SEQ ID NO: 452 - DNA, 20 bp, Artificial Sequence
  Description of Artificial Sequence Synthetic oligonucleotide
ctggtttgcttaaaaatgtg

SEQ ID NO: 453 - DNA, 22 bp, Artificial Sequence
  Description of Artificial Sequence Synthetic oligonucleotide
tttcaaaagtgataaattttaa

SEQ ID NO: 454 - DNA, 23 bp, Artificial Sequence
  Description of Artificial Sequence Synthetic oligonucleotide
aaaagtgataaattttaaataca

SEQ ID NO: 455 - DNA, 23 bp, Artificial Sequence
  Description of Artificial Sequence Synthetic oligonucleotide
cttaaaaatgtgtgtatttaaaa

SEQ ID NO: 456 - DNA, 23 bp, Artificial Sequence
  Description of Artificial Sequence Synthetic oligonucleotide
aaaagtgataaattttaaataca

SEQ ID NO: 457 - DNA, 23 bp, Artificial Sequence
  Description of Artificial Sequence Synthetic oligonucleotide
gcttaaaaatgtgtgtatttaaa

SEQ ID NO: 458 - DNA, 23 bp, Artificial Sequence
  Description of Artificial Sequence Synthetic oligonucleotide
cttaaaaatgtgtgtatttaaaa

SEQ ID NO: 459 - DNA, 21 bp, Artificial Sequence
  Description of Artificial Sequence Synthetic oligonucleotide
aattttaaatacacacatttt

SEQ ID NO: 460 - DNA, 23 bp, Artificial Sequence
  Description of Artificial Sequence Synthetic oligonucleotide
aaagtgataaattttaaatacac

SEQ ID NO: 461 - DNA, 23 bp, Artificial Sequence
  Description of Artificial Sequence Synthetic oligonucleotide
ctggtttgcttaaaaatgtgtgt

SEQ ID NO: 462 - DNA, 21 bp, Artificial Sequence
  Description of Artificial Sequence Synthetic oligonucleotide
ttgcttaaaaatgtgtgtatt

SEQ ID NO: 463 - DNA, 20 bp, Artificial Sequence
  Description of Artificial Sequence Synthetic oligonucleotide
tggatgaagttggtggtgag

SEQ ID NO: 464 - DNA, 21 bp, Artificial Sequence
  Description of Artificial Sequence Synthetic primer
aaacatcaagcgtcccataga

SEQ ID NO: 465 - DNA, 23 bp, Artificial Sequence
  Description of Artificial Sequence Synthetic primer
tgaccaaatcagggtaattttgc

SEQ ID NO: 466 - DNA, 20 bp, Artificial Sequence
  Description of Artificial Sequence Synthetic primer
caggagctgtgggaggaaga

SEQ ID NO: 467 - DNA, 21 bp, Artificial Sequence
  Description of Artificial Sequence Synthetic primer
caccttatattcccagggccg

SEQ ID NO: 468 - DNA, 21 bp, Artificial Sequence
  Description of Artificial Sequence Synthetic primer
cctaggacgcaccattctcac

SEQ ID NO: 469 - DNA, 20 bp, Artificial Sequence
  Description of Artificial Sequence Synthetic primer
attgggtctaacccccacct

SEQ ID NO: 470 - DNA, 20 bp, Artificial Sequence
  Description of Artificial Sequence Synthetic primer
tcagtgaaacgcaccagaca

SEQ ID NO: 471 - DNA, 25 bp, Artificial Sequence
  Description of Artificial Sequence Synthetic primer
ctcctgaggagaagtctgccgttac

SEQ ID NO: 472 - DNA, 19 bp, Artificial Sequence
  Description of Artificial Sequence Synthetic primer
gcagctcactcagtgtggc

SEQ ID NO: 473 - DNA, 20 bp, Artificial Sequence
  Description of Artificial Sequence Synthetic primer
tgggcaggttggtatcaagg

SEQ ID NO: 474 - DNA, 20 bp, Artificial Sequence
  Description of Artificial Sequence Synthetic primer
gcactttcttgccatgagcc

SEQ ID NO: 475 - DNA, 27 bp, Artificial Sequence
  Description of Artificial Sequence Synthetic primer
ctctttctttcagggcaataatgatac

SEQ ID NO: 476 - DNA, 22 bp, Artificial Sequence
  Description of Artificial Sequence Synthetic primer
ccagccttatcccaaccataaa

SEQ ID NO: 477 - DNA, 23 bp, Artificial Sequence
  Description of Artificial Sequence Synthetic primer
gcaaggtgaacgtggatgaagtt

SEQ ID NO: 478 - DNA, 23 bp, Artificial Sequence
  Description of Artificial Sequence Synthetic primer
ggacagatccccaaaggactcaa

SEQ ID NO: 479 - DNA, 21 bp, Artificial Sequence
  Description of Artificial Sequence Synthetic primer
tgaggagaagtctgccgttac

SEQ ID NO: 480 - DNA, 20 bp, Artificial Sequence
  Description of Artificial Sequence Synthetic primer
caccagccaccactttctga

SEQ ID NO: 481 - DNA, 21 bp, Artificial Sequence
  Description of Artificial Sequence Synthetic primer
tgaggagaagtctgccgttac

SEQ ID NO: 482 - DNA, 19 bp, Artificial Sequence
  Description of Artificial Sequence Synthetic primer
accaccagcagcctgccca

SEQ ID NO: 483 - DNA, 18 bp, Artificial Sequence
  Description of Artificial Sequence Synthetic primer
ttcaggctcctgggcaac

SEQ ID NO: 484 - DNA, 20 bp, Artificial Sequence
  Description of Artificial Sequence Synthetic primer
caccagccaccactttctga

SEQ ID NO: 485 - DNA, 18 bp, Artificial Sequence
  Description of Artificial Sequence Synthetic primer
gccctggagaggatgttc

SEQ ID NO: 486 - DNA, 18 bp, Artificial Sequence
  Description of Artificial Sequence Synthetic primer
ttcttgccgtggccctta

SEQ ID NO: 487 - DNA, 23 bp, Artificial Sequence
  Description of Artificial Sequence Synthetic primer
ggttatcaataagctcctagtcc

SEQ ID NO: 488 - DNA, 19 bp, Artificial Sequence
  Description of Artificial Sequence Synthetic primer
acaaccaggagccttccca

SEQ ID NO: 489 - DNA, 22 bp, Artificial Sequence
  Description of Artificial Sequence Synthetic primer
gaggagaagactgctgtcaatg

SEQ ID NO: 490 - DNA, 22 bp, Artificial Sequence
  Description of Artificial Sequence Synthetic primer
agggtagaccaccagtaatctg

SEQ ID NO: 491 - DNA, 27 bp, Artificial Sequence
  Description of Artificial Sequence Synthetic oligonucleotide
  modified_base (5)..(27): a, c, t, or g
tttvnnnnnnnnnnnnnnnnnnnnnnn

SEQ ID NO: 492 - DNA, 20 bp, Artificial Sequence
  Description of Artificial Sequence Synthetic oligonucleotide
  modified_base (5)..(20): a, c, t, or g
tttvnnnnnnnnnnnnnnnn

SEQ ID NO: 493 - DNA, 27 bp, Artificial Sequence
  Description of Artificial Sequence Synthetic oligonucleotide
  modified_base (5)..(27): a, c, t, or g
tttvnnnnnnnnnnnnnnnnnnnnnnn

SEQ ID NO: 494 - DNA, 40 bp, Homo sapiens
actgactctctctgcctattagtctattttcccaccctta

SEQ ID NO: 495 - DNA, 40 bp, Homo sapiens
atttctgggttaaggtaatagcaatatttctgcatataaa